(19) United States
(12) Patent Application Publication
(10) Pub. No.: US 2024/0376066 A1
(43) Pub. Date: Nov. 14, 2024

(54) COMPOUND FOR ORGANIC ELECTRIC ELEMENT, ORGANIC ELECTRIC ELEMENT USING THE SAME, AND AN ELECTRONIC DEVICE THEREOF

(71) Applicant: DUK SAN NEOLUX CO., LTD., Cheonan-si, Chungcheongnam-do (KR)

(72) Inventors: Yeon Seok JEONG, Cheonan-si, Chungcheongnam-do (KR); Jong Gwang PARK, Cheonan-si, Chungcheongnam-do (KR); Jae Duk YOO, Cheonan-si, Chungcheongnam-do (KR)

(73) Assignee: DUK SAN NEOLUX CO., LTD., Cheonan-si, Chungcheongnam-do (KR)

(21) Appl. No.: 18/688,208

(22) PCT Filed: Jul. 15, 2022
(86) PCT No.: PCT/KR2022/010397

§ 371 Date: Feb. 29, 2024

(30) Foreign Application Priority Data
Sep. 1, 2021 (KR) 10-2021-0116457
Jun. 28, 2022 (KR) 10-2022-0078669

(51) Int. Cl. C07D 333/76; C07B 59/00; C07D 307/91; C07D 405/14; C07D 407/12; C07D 407/14; C07D 409/12; C07D 409/14; C07D 413/12; C09K 11/06; H10K 50/15; H10K 85/60

(52) U.S. Cl. CPC C07D 333/76; C07B 59/004; C07D 307/91; C07D 405/14; C07D 407/12; C07D 407/14; C07D 409/12; C07D 409/14; C07D 413/12; C09K 11/06; H10K 50/15; H10K 85/622; H10K 85/633; H10K 85/636; H10K 85/653; H10K 85/654; H10K 85/6572; H10K 85/6574; H10K 85/6576; C07B 2200/05

(57) ABSTRACT

Provided are a compound represented by Formula 1, an organic electric element comprising a first electrode, a second electrode, and an organic material layer between the first electrode and the second electrode comprising a compound represented by Formula 1, and an electronic device thereof, wherein the driving voltage of the organic electric element can be lowered, and the luminous efficiency and life time of the organic electric element can be improved by including the compound represented by Formula 1 in the organic material layer.

### BACKGROUND

### Technical Field

The present invention relates to compound for organic electric element, organic electric element using the same, and electronic device thereof.

### Background Art

In general, an organic light emitting phenomenon refers to a phenomenon in which electric energy is converted into light energy of an organic material. An organic electric element utilizing the organic light emitting phenomenon usually has a structure including an anode, a cathode, and an organic material layer interposed therebetween. In many cases, the organic material layer has a multi-layered structure having respectively different materials in order to improve efficiency and stability of an organic electric element, and for example, may comprise a hole injection layer, a hole transport layer, a light emitting layer, an electron transport layer, an electron injection layer, or the like.

Materials used as an organic material layer in an organic electric element may be classified into a light emitting material and a charge transport material, for example, a hole injection material, a hole transport material, an electron transport material, an electron injection material, and the like according to its function. Further, the light emitting material may be divided into a high molecular weight type and a low molecular weight type according to its molecular weight, and may also be divided into a fluorescent material derived from excited singlet states of electron and a phosphorescent material derived from excited triplet states of electron according to its light emitting mechanism. Further, the light emitting material may be divided into blue, green, and red light emitting material and yellow and orange light emitting material required for better natural color reproduction according to its light emitting color.

Meanwhile, when only one material is used as a light emitting material, there occur problems of shift of a maximum luminescence wavelength to a longer wavelength due to intermolecular interactions and lowering of the efficiency of a corresponding element due to deterioration in color purity or a reduction in luminous efficiency. On account of this, a host/dopant system may be used as the light emitting material in order to enhance the color purity and increase the luminous efficiency through energy transfer. This is based on the principle that if a small amount of dopant having a smaller energy band gap than a host forming a light emitting layer is mixed in the light emitting layer, then excitons generated in the light emitting layer are transported to the dopant, thus emitting light with high efficiency. With regard to this, since the wavelength of the host is shifted to the wavelength band of the dopant, light having a desired wavelength can be obtained according to the type of the dopant.

In addition, recently, a method of using an emission-auxiliary layer between a hole transport layer and a light emitting layer has been studied in order to solve the problem of light emission from the hole transport layer in organic electroluminescent devices, and it is required to develop an emission-auxiliary layer according to each light-emitting layer since required material properties are different depending on each light-emitting layer (R, G, and B).

Currently, the power consumption is required more than more as size of display becomes larger and larger in the portable display market. Therefore, the power consumption is very important factor in the portable display with a limited power source of the battery, and efficiency and life span issues are also solved.

Efficiency, life span, driving voltage, and the like are correlated with each other. If efficiency is increased, then driving voltage is relatively lowered, and the crystallization of an organic material due to Joule heating generated during operation is reduced as driving voltage is lowered. As a result, life span tens to increase.

However, efficiency cannot be maximized only by simply improving the organic material layer. This is because long life span and high efficiency can be simultaneously achieved when an optimal combination of energy levels and Ti values, inherent material properties (mobility, interfacial properties, etc.), and the like among the respective layers included in the organic material layer is given. Therefore, it is necessary to develop materials for a light-emitting layer and an emission-auxiliary layer that can improve both the efficiency and lifespan of the device.

### Object, Technical Solution and Effects of the Invention

The object of the present invention is to provide a compound capable of lowering the driving voltage of the element and improving the luminous efficiency and life time, an organic electric element using the same, and an electronic device thereof.

In an aspect of the present invention, the present invention provides compound represented by the following formula.

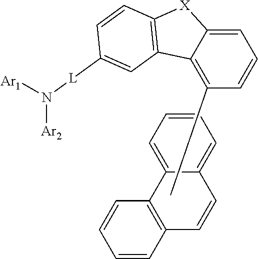

In another aspect of the present invention, the present invention provides an organic electric element employing compound represented by formula above and an electronic device thereof.

By using the compound according to embodiment of the present invention, a driving voltage of an organic electric element can be lowered and the luminous efficiency and lifetime of the element can be improved.

### BRIEF DESCRIPTION OF THE DRAWINGS

FIGS. 1 to 3 illustrate an example of organic electroluminescent element according to the present invention.

[Description of Reference Numerals]

| | |
|---|---|
| 100, 200, 300: organic electric element | 110: first electrode |
| 120: hole injection layer | 130: hole transport layer |
| 140: light emitting layer | 150: electron transport layer |
| 160: electron injection layer | 170: second electrode |
| 180: light efficiency improving layer | 210: buffer layer |
| 220: emission-auxiliary layer | 320: first hole injection layer |
| 330: first hole transport layer | 340: first light emitting layer |
| 350: first electron transport layer | 360: first charge generation layer |
| 361: second charge generation layer | 420: second hole injection layer |
| 430: second hole transport layer | 440: second light emitting layer |
| 450: second electron transport layer | CGL: charge generation layer |
| ST1: first stack | ST2: second stack |

### DETAILED DESCRIPTION

Unless otherwise stated, the term “aryl group” or “arylene group” as used herein has, but not limited to, 6 to 60 carbon atoms. The aryl group or arylene group in the present invention may comprise a monocyclic ring, ring assemblies, a fused polycyclic system, spiro compound and the like.

As used herein, the term “fluorenyl group” refers to a substituted or unsubstituted fluorenyl group, and “fluorenylene group” refers to a substituted or unsubstituted fluorenylene group. The fluorenyl group or fluorenylene group used in the present invention comprises a spiro compound formed by combining R and R′ with each other in the following structure, and also comprises compound formed by linking adjacent R″s to each other. “Substituted fluorenyl group”, “substituted fluorenylene group” means that at least one of R, R′, R″ in the following structure is a substituent other than hydrogen, and R″ may be 1 to 8 in the following formula. In this specification, a fluorene group and a fluoreneylene group may be referred to as a fluorene group or fluorene regardless of the valence.

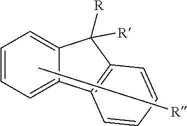

The term “spiro compound” as used herein has a spiro union which means union having one atom as the only common member of two rings. The common atom is designated as ‘spiro atom’. The compounds are defined as ‘monospiro-’, ‘dispiro-’ or ‘trispiro-’ depending on the number of spiro atoms in one compound.

The term “heterocyclic group” used in the specification comprises a non-aromatic ring as well as an aromatic ring like “heteroaryl group” or “heteroarylene group”. Unless otherwise stated, the term “heterocyclic group” means, but not limited to, a ring containing one or more heteroatoms and having 2 to 60 carbon atoms. Unless otherwise stated, the term “heteroatom” as used herein represents, for example, N, O, S, P or Si and may comprise a heteroatom group such as SO₂, P═O etc. instead of carbon forming a ring such as the following compound. In the specification, “heterocyclic group” comprises a monocyclic, ring assemblies, fused polycyclic system, a spiro-compound and the like.

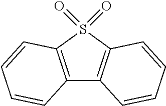 [SMILES: O=S1(=O)c2ccccc2-c2ccccc21]

The term “aliphatic ring group” as used herein refers to a cyclic hydrocarbon except for aromatic hydrocarbons, and comprises a monocyclic ring, ring assemblies, a fused polycyclic system, a spiro-compound and the like, and unless otherwise specified, it means a ring of 3 to 60 carbon atoms, but not limited thereto. For example, a fused ring of benzene and cyclohexane corresponds to aliphatic ring group, wherein benzene is an aromatic ring and cyclohexane is a non-aromatic ring.

In this specification, a ‘group name’ corresponding to an aryl group, an arylene group, a heterocyclic group, and the like exemplified for each symbol and its substituent may be written in the name of functional group reflecting the valence, and may also be described as the name of a parent compound. For example, in the case of phenanthrene which is a kind of aryl group, it may be described by distinguishing valence such as ‘phenanthryl (group)’ when it is ‘monovalent group’, and ‘phenanthrylene (group)’ when it is ‘divalent group’, and it may also be described as a parent compound name, ‘phenanthrene’, regardless of its valence. Similarly, in the case of pyrimidine, it may be described as ‘pyrimidine’ regardless of its valence, and it may also be described as the name of corresponding functional group such as pyrimidinyl (group) when it is ‘monovalent group’, and ‘pyrimidylene (group)’ when it is ‘divalent group’.

In addition, in the present specification, the numbers and alphabets indicating a position may be omitted when describing a compound name or a substituent name. For example, pyrido[4,3-d]pyrimidine, benzofuro[2,3-d]pyrimidine and 9,9-dimethyl-9H-fluorene can be described as pyridopyrimidine, benzofuropyrimidine and dimethylfluorene, respectively. Therefore, both benzo[g]quinoxaline and benzo[f]quinoxaline can be described as benzoquinoxaline.

In addition, unless otherwise expressed, where any formula of the present invention is represented by the following formula, the substituent according to the index may be defined as follows.

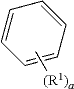

In the above formula, where a is an integer of zero, the substituent R¹ is absent, that is, hydrogen atoms are bonded to all the carbon constituting the benzene ring. Here, chemical formulas or compounds may be written without explicitly describing the hydrogen.

Here, when a is an integer of 0, the substituent R¹ is absent, which means that all hydrogen is bonded to the carbons forming the benzene ring, and is the same as the case where R¹ is hydrogen and a is an integer of 1 to 5. At this time, description of hydrogen bonded to carbon may be omitted.

In addition, one substituent R¹ is bonded to any carbon of the carbons forming the benzene ring when “a” is an integer of 1. Similarly, where “a” is an integer of 2 or 3, substituents R's may be bonded to the carbon of the benzene ring, for example, as followings. Also, where “a” is an integer of 4 to 6, substituents R's are bonded to the carbon of the benzene ring in a similar manner. Further, where “a” is an integer of 2 or more, R¹s may be the same or different from each other.

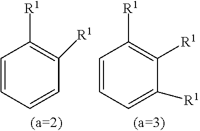

In addition, unless otherwise specified in the present specification, when referring to a condensed/fused ring, the number in the ‘number-condensed/fused ring’ indicates the number of condensed/fused rings. For example, a form in which three rings are condensed/fused with each other, such as anthracene, phenanthrene, and benzoquinazoline, may be represented by a 3-condensed/fused ring.

In addition, unless otherwise described herein, in the case of expressing a ring in the form of a ‘number-membered’ such as a 5-membered ring or a 6-membered ring, the number in the ‘number-membered’ represents the number of atoms forming the ring. For example, thiophene or furan may correspond to a 5-membered ring, and benzene or pyridine may correspond to a 6-membered ring.

In addition, unless otherwise specified in the present specification, when adjacent groups are linked to each other to form a ring, the ring may be selected from the group consisting of a C₆-C₆₀ aromatic ring group, a fluorenyl group, a C₂-C₆₀ heterocyclic group containing at least one heteroatom selected from the group consisting of O, N, S, Si and P, and a C₃-C₆₀ aliphatic ring.

Unless otherwise stated, the term “between adjacent groups”, for example, in case of the following Formulas, comprises not only “between R₁ and R₂”, “between R₂ and R₃”, “between R₃ and R₄”, “between R₅ and R₆”, but also “between R₇ and R₈” sharing one carbon, and may comprise “between substituents” attached to atom (carbon or nitrogen) consisting different ring, such as “between R₁ and R₇”, “between R₁ and R₈”, or “between R₄ and R₅” and the like. That is, where there are substituents bonded to adjacent elements constituting the same ring, the substituents may be correspond “adjacent groups”, and even if there are no adjacent substituents on the same ring, substituents attached to the adjacent ring may correspond to “adjacent groups”. In the following Formula, when the substituents bonded to the same carbon, such as R₇ and R₈, are linked to each other to form a ring, a compound containing a spiro-moiety may be formed.

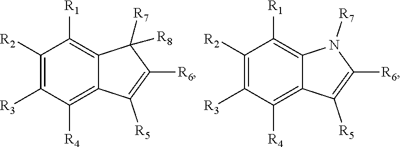

In addition, in the present specification, the expression ‘adjacent groups may be linked to each other to form a ring’ is used in the same sense as ‘adjacent groups are linked selectively to each other to form a ring’, and a case where at least one pair of adjacent groups may be bonded to each other to form a ring.

In addition, unless otherwise specified in the present specification, an aryl group, an arylene group, a fluorenyl group, a fluorenylene group, a heterocyclic group, an aliphatic ring group, an alkyl group, an alkenyl group, an alkynyl group, an alkoxyl group, an aryloxyl group, and a ring formed by adjacent groups may be each optionally substituted with one or more substituents selected from the group consisting of deuterium, halogen, an amino group unsubstituted or substituted with a C₁-C₂₀ alkyl group or a C₆-C₂₀ aryl group, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₂₀ aryl group, a fluorenyl group, a C₂-C₂₀ heterocyclic group containing at least one heteroatom of O, N, S, Si and P, and a C₃-C₂₀ aliphatic ring group.

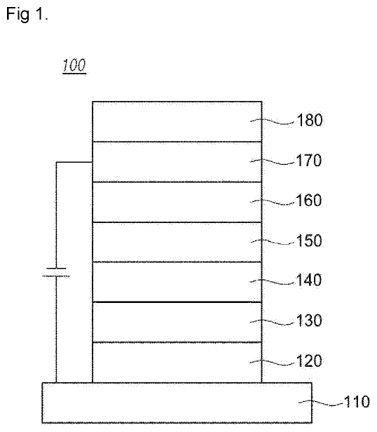

Hereinafter, referring to FIGS. 1 to 3, a lamination structure of an organic electric element including the compound of the present invention will be described.

In designation of reference numerals to components in respective drawings, it should be noted that the same elements will be designated by the same reference numerals although they are shown in different drawings. In addition, in the following description of the present invention, a detailed description of known functions and configurations incorporated herein will be omitted when it may make the subject matter of the present invention rather unclear.

Terms, such as first, second, A, B, (a), (b) or the like may be used herein when describing components of the present invention. Each of these terminologies is not used for defining an essence, order or sequence of a corresponding component but used merely to distinguish the corresponding component from other component(s). It will be understood that the expression ‘one component is “connected,” “coupled” or “joined” to another component’ comprises the case where a third component may be “connected,” “coupled” or “joined” between a first component and a second component as well as the case where the first component may be directly connected, coupled or joined to the second component.

In addition, it will be understood that when an element such as a layer, film, region or substrate is referred to as being “on” or “over” another element, it can be directly on the other element or intervening elements may also be present. In contrast, when an element is referred to as being “directly on” another element, there are no intervening elements present.

The FIGS. 1 to 3 show an example of an organic electric element according to an embodiment of the present invention, respectively.

Referring to the FIG. 1, an organic electric element 100 according to an embodiment of the present invention includes a first electrode 110 formed on a substrate (not shown), a second electrode 170, and an organic material layer between the first electrode 110 and the second electrode 170.

The first electrode 110 may be an anode (positive electrode), and the second electrode 170 may be a cathode (negative electrode). In the case of an inverted organic electric element, the first electrode may be a cathode, and the second electrode may be an anode.

The organic material layer may be comprised a hole injection layer 120, a hole transport layer 130, a light emitting layer 140, an electron transport layer 150, and an electron injection layer 160. Specifically, a hole injection layer 120, a hole transport layer 130, a light emitting layer 140, an electron transport layer 150, and an electron injection layer 160 may be formed on the first electrode 110 in sequence.

Preferably, a layer for improving the luminous efficiency 180 may be formed one side of sides of the first electrode 110 and the second electrode 170, wherein one side is not facing the organic material layer, as a result the luminous efficiency of an organic electric element can be improved.

For example, the light efficiency improving layer 180 may be formed on the second electrode 170, as a result, in the case of a top emission organic light emitting element, the optical energy loss due to Surface Plasmon Polaritons (SPPs) at the second electrode 170 may be reduced and in the case of a bottom emission organic light emitting element, the light efficiency improving layer 180 may serve as a buffer for the second electrode 170.

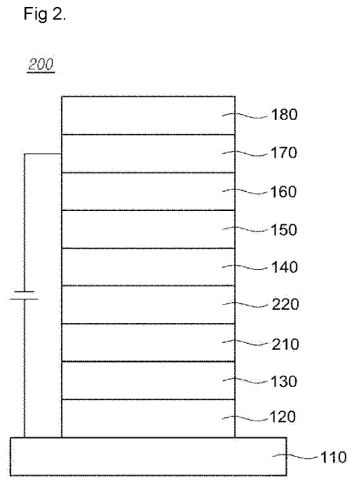

A buffer layer 210 or an emission-auxiliary layer 220 may be further formed between the hole transport layer 130 and the light emitting layer 140, which will be described with reference to FIG. 2.

Referring to FIG. 2, the organic electric element 200 according to another embodiment of the present invention may comprise a hole injection layer 120, a hole transport layer 130, a buffer layer 210, an emission-auxiliary layer 220, a light emitting layer 140, the electron transport layer 150, the electron injection layer 160, and a second electrode 170 formed on a first electrode 110 in sequence, and a layer for improving light efficiency 180 may be formed on the second electrode 170.

Although not shown in FIG. 2, an electron transport auxiliary layer may be further formed between the light emitting layer 140 and the electron transport layer 150.

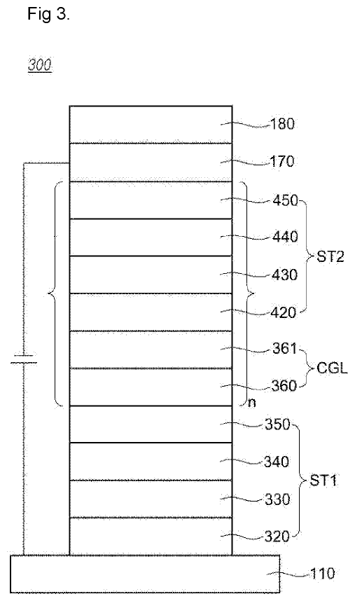

In addition, according to another embodiment of the present invention, the organic material layer may be a form consisting of multiple stacks, wherein the stacks comprise a hole transport layer, a light emitting layer, and an electron transport layer, respectively. This will be described with reference to FIG. 3.

Referring to FIG. 3, two or more sets of stacks of the organic material layers ST1 and ST2 may be formed between the first electrode 110 and the second electrode 170 in the organic electric element 300 according to another embodiment of the present invention, wherein the organic material layers are consisted of multiple layers, respectively, and the charge generation layer CGL may be formed between the stacks of the organic material layer.

Specifically, the organic electric element according to the embodiment of the present invention may comprise a first electrode 110, a first stack ST1, a charge generation layer CGL, a second stack ST2, and a second electrode 170 and a layer for improving light efficiency 180.

The first stack ST1 is an organic layer formed on the first electrode 110, and the first stack ST1 may comprise the first hole injection layer 320, the first hole transport layer 330, the first light emitting layer 340 and the first electron transport layer 350 and the second stack ST2 may comprise a second hole injection layer 420, a second hole transport layer 430, a second light emitting layer 440 and a second electron transport layer 450. As such, the first stack and the second stack may be the organic layers having the same or different stacked structures.

The charge generation layer CGL may be formed between the first stack ST1 and the second stack ST2. The charge generation layer CGL may comprise a first charge generation layer 360 and a second charge generation layer 361. The charge generating layer CGL is formed between the first light emitting layer 340 and the second light emitting layer 440 to increase the current efficiency generated in each light emitting layer and to smoothly distribute charges.

The first light emitting layer 340 may comprise a light emitting material comprising a blue host doped with a blue fluorescent dopant and the second light emitting layer 440 may comprise a light emitting material comprising a green host doped with a greenish yellow dopant and a red dopant together, but the material of the first light emitting layer 340 and the second light emitting layer 440 according to an embodiment of the present invention is not limited thereto.

In FIG. 3, n may be an integer of 1 to 5 and the charge generation layer CGL and the third stack may be further stacked on the second stack ST2 when n is 2.

When multiple light emitting layers are formed in a multi-layer stack structure as shown in FIG. 3, it is possible to manufacture an organic electroluminescent element that emits not only white light but also various colors, wherein the white light is emitted by the mixing effect of light emitted from each light emitting layer.

Compound represented by Formula 1 may be comprised in an organic material layer. For example, compound represented by Formula 1 may be used as material of a hole injection layer 120, 320, 420, a hole transport layer 130, 330, 430, a buffer layer 210, an emission-auxiliary layer 220, an electron transport layer 150, 350, 450, an electron injection layer 160, a light emitting layer 140, 340, 440 or a layer for improving light efficiency 180, preferably, an emission-auxiliary layer 220, a light emitting layer 140, 340, 440 or/and a layer for improving light efficiency 180, more preferably, an emission-auxiliary layer 220 and/or a light emitting layer 140, 340, 440.

Even if the cores of compounds are identical or similar to each other, the band gap, the electrical characteristics, the interface characteristics and the like may be different depending on which substituent is bonded at which position. Therefore, it is necessary to study the selection of the core and the combination with sub-substituent bonded to the core. In particular, long life span and high efficiency can be simultaneously achieved when the optimal combination of energy levels and Ti values, inherent material properties (mobility, interfacial properties, etc.), and the like among the respective layers of an organic material layer is achieved.

Therefore, energy level and Ti value between the respective layers of the organic material layer, inherent material properties (mobility, interfacial properties, etc.) and the like can be optimized by using compound represented by Formula 1 as material of an emission-auxiliary layer 220 and/or a light emitting layer 140, 340, 440, and thus it is possible to simultaneously improve the lifetime and efficiency of the organic electric element.

The organic electric element according to an embodiment of the present invention may be manufactured using various deposition methods. The organic electric element according to an embodiment of the present invention may be manufactured using a PVD (physical vapor deposition) method or CVD (chemical vapor deposition) method. For example, the organic electric element may be manufactured by depositing a metal, a conductive metal oxide, or a mixture thereof on the substrate to form the anode 110, forming the organic material layer comprising the hole injection layer 120, the hole transport layer 130, the light emitting layer 140, the electron transport layer 150, and the electron injection layer 160 thereon, and then depositing a material, which can be used as the cathode 170, thereon. Also, an emission-auxiliary layer 220 may be formed between a hole transport layer 130 and a light emitting layer 140, and an electron transport auxiliary layer (not shown) may be further formed between a light emitting layer 140 and an electron transport layer 150 and, as described above, a stack structure may be formed.

Also, the organic material layer may be manufactured in such a manner that the fewer layers are formed using various polymer materials by a soluble process or solvent process, for example, spin coating, nozzle printing, inkjet printing, slot coating, dip coating, roll-to-roll, doctor blading, screen printing, or thermal transfer, instead of deposition. Since the organic material layer according to the present invention may be formed in various ways, the scope of protection of the present invention is not limited by a method of forming the organic material layer.

The organic electric element according to an embodiment of the present invention may be of a top emission type, a bottom emission type, or a dual emission type depending on the material used.

In addition, the organic electric element according to an embodiment of the present invention may be selected from the group consisting of an organic electroluminescent element, an organic solar cell, an organic photo conductor, an organic transistor, an element for monochromatic illumination and an element for quantum dot display.

Another embodiment of the present invention provides an electronic device including a display device which includes the above described organic electric element, and a control unit for controlling the display device. Here, the electronic device may be a wired/wireless communication terminal which is currently used or will be used in the future, and covers all kinds of electronic devices including a mobile communication terminal such as a cellular phone, a navigation unit, a game player, various kinds of TVs, and various kinds of computers.

Hereinafter, compound according to one aspect of the present invention will be described.

Compound according to an aspect of the present invention is represented by Formula 1 below.

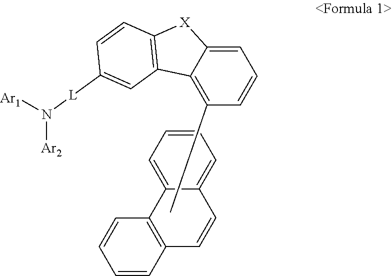

In Formula 1, each of symbols may be defined as follows.

X is O or S.

Ar₁ and Ar₂ are each independently selected from the group consisting of a C₆-C₆₀ aryl group, a fluorenyl group, a C₂-C₆₀ heterocyclic group containing at least one heteroatom of O, N, S, Si and P, and a C₃-C₆₀ aliphatic ring group. Preferably, both Ar₁ and Ar₂ may be a C₆-C₆₀ aryl group, or one of them may be a C₆-C₆₀ aryl group and the other may be a fluorenyl group.

L is selected from the group consisting of a single bond, a C₆-C₆₀ arylene group, a fluorenylene group, a C₂-C₆₀ heterocyclic group containing at least one heteroatom of O, N, S, Si and P and a C₃-C₆₀ aliphatic ring group.

The aryl group, the arylene group, the fluorenyl group, the fluorenylene group, the heterocyclic group, and the aliphatic ring group may be each optionally substituted with one or more substituents selected from the group consisting of deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group containing at least one heteroatom of O, N, S, Si and P, a C₃-C₃₀ aliphatic ring group and -L′-N(Rₐ)(R_{b}).

L′ is selected from the group consisting of a single bond, a C₆-C₃₀ arylene group, a fluorenylene group, a C₂-C₃₀ heterocyclic group containing at least one heteroatom of O, N, S, Si and P and a C₃-C₃₀ aliphatic ring group.

Rₐ and R_{b} are each independently selected from the group consisting of a C₆-C₃₀ aryl group, a fluorenyl group, a C₂-C₃₀ heterocyclic group containing at least one heteroatom of O, N, S, Si and P, and a C₃-C₃₀ aliphatic ring group.

When at least one of Ar₁, Ar₂, Rₐ and R_{b} is an aryl group, the aryl group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, a C₁₈, a C₁₉, a C₂₀, a C₂₁, a C₂₂, a C₂₃, a C₂₄, a C₂₅, a C₂₆, a C₂₇, a C₂₈, a C₂₉, a C₃₀ aryl group, specifically, phenyl, biphenyl, naphthyl, terphenyl, phenanthrene, triphenylene, chrysene or the like.

When at least one of L and L′ is an arylene group, the arylene group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, a C₁₈, a C₁₉, a C₂₀, a C₂₁, a C₂₂, a C₂₃, a C₂₄, a C₂₅, a C₂₆, a C₂₇, a C₂₈, a C₂₉, a C₃₀ arylene group, specifically, phenylene, biphenyl, naphthylene, terphenyl, phenanthrene, triphenylene, or the like.

When at least one of Ar₁, Ar₂, Rₐ, R_{b}, L and L′ is a heterocyclic group, the heterocyclic group may be, for example, a C₂-C₃₀, a C₂-C₂₉, a C₂-C₂₈, a C₂-C₂₇, a C₂-C₂₆, a C₂-C₂₅, a C₂-C₂₄, a C₂-C₂₃, a C₂-C₂₂, a C₂-C₂₁, a C₂-C₂₀, a C₂-C₁₉, a C₂-C₁₈, a C₂-C₁₇, a C₂-C₁₆, a C₂-C₁₅, a C₂-C₁₄, a C₂-C₁₃, a C₂-C₁₂, a C₂-C₁₁, a C₂-C₁₀, a C₂-C₉, a C₂-C₈, a C₂-C₇, a C₂-C₆, a C₂-C₅, a C₂-C₄, a C₂-C₃, a C₂, a C₃, a C₄, a C₅, a C₆, a C₇, a C₈, a C₉, a C₁₀, a C₁₁, a C₁₂, a C₁₃, a C₁₄, a C₁s, a C₁₆, a C₁₇, a C₁₈, a C₁₉, a C₂₀, a C₂₁, a C₂₂, a C₂₃, a C₂₄, a C₂₅, a C₂₆, a C₂₇, a C₂₈, a C₂₉ or C₃₀ heterocyclic group, specifically, pyridine, pyrimidine, pyrazine, pyridazine, triazine, furan, pyrrole, silole, indene, indole, phenyl-indole, benzoindole, phenyl-benzoindole, pyrazinoindol, quinoline, isoquinoline, benzoquinoline, pyridoquinoline, quinazoline, benzoquinazoline, dibenzoquinazoline, phenanthroquinazoline, quinoxaline, benzoquinoxaline, dibenzoquinoxaline, benzofuran, naphthobenzofuran, dibenzofuran, dinaphthofuran, thiophene, benzothiophene, dibenzothiophene, naphthobenzothiophene, dinaphthothiophene, carbazole, phenyl-carbazole, benzocarbazole, phenyl-benzocarbazole, naphthyl-benzocarbazole, dibenzocarbazole, indolocarbazole, benzofuropyridine, benzothienopyridine, benzofuropyridine, benzothienopyrimidine, benzofuropyrimidine, benzothienopyrazine, benzofuropyrazine, benzoimidazole, benzothiazole, benzooxazole, naphthooxazole, phenanthrooxazole, benzosiloe, phenanthroline, dihydro-phenylphenazine, 10-phenyl-10H-phenoxazine, phenoxazine, phenothiazine, dibenzodioxin, benzodibenzodioxin, thianthrene, 9,9-dimethyl-9H-xantene, 9,9-dimethyl-9H-thioxantene, dihydrodimethylphenylacridine, spiro[fluorene-9,9′-xanthene] and the like.

When at least one of Ar₁, Ar₂, Rₐ and R_{b} is a fluorenyl group or at least one of L and L′ is a fluorenylene group, the fluorenyl group or the fluorenylene group may be, for example, 9,9-dimethyl-9H-fluorene, 9,9-diphenyl-9H-fluorene, 9,9′-spirobifluorene, spiro[benzo[b]fluorene-11,9′-fluorene], benzo[b]fluorene, 11,11-diphenyl-11H-benzo[b]fluorene, 9-(naphthalen-2-yl)9-phenyl-9H-fluorene, and the like.

When at least one of the aryl group, the fluorenyl group, the heterocyclic group, the aliphatic ring group, the arylene group and the fluorenylene group is substituted with an aryl group, the aryl group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, a C₁₈, a C₁₉, a C₂₀, a C₂₁, a C₂₂, a C₂₃, a C₂₄, a C₂₅, a C₂₆, a C₂₇, a C₂₈, a C₂₉, or a C₃₀ aryl group.

When at least one of the aryl group, the fluorenyl group, the heterocyclic group, the aliphatic ring group, the arylene group and the fluorenylene group is substituted with a heterocyclic group, the heterocyclic group may be, for example, a C₂-C₃₀, a C₂-C₂₉, a C₂-C₂₈, a C₂-C₂₇, a C₂-C₂₆, a C₂-C₂₅, a C₂-C₂₄, a C₂-C₂₃, a C₂-C₂₂, a C₂-C₂₁, a C₂-C₂₀, a C₂-C₁₉, a C₂-C₁₈, a C₂-C₁₇, a C₂-C₁₆, a C₂-C₁₅, a C₂-C₁₄, a C₂-C₁₃, a C₂-C₁₂, a C₂-C₁₁, a C₂-C₁₀, a C₂-C₉, a C₂-C₈, a C₂-C₇, a C₂-C₆, a C₂-C₅, a C₂-C₄, a C₂-C₃, a C₂, a C₃, a C₄, a C₅, a C₆, a C₇, a C₈, a C₉, a C₁₀, a C₁, a C₁₂, a C₁₃, a C₁₄, a C₁₅, a C₁₆, a C₁₇, a C₁₈, a C₁₉, a C₂₀, a C₂₁, a C₂₂, a C₂₃, a C₂₄, a C₂₅, a C₂₆, a C₂₇, a C₂₈, a C₂₉ or C₃₀ heterocyclic group.

When at least one of the aryl group, the fluorenyl group, the heterocyclic group, the aliphatic ring group, the arylene group and the fluorenylene group is substituted with a fluorenyl group, the fluorenyl group may be 9,9-dimethyl-9H-fluorene, 9,9-diphenyl-9H-fluorene, 9,9′-spirobifluorene, spiro[benzo[b]fluorene-11,9′-fluorene], benzo[b]fluorene, 11,11-diphenyl-11H-benzo[b]fluorene, 9-(naphthalen-2-yl)9-phenyl-9H-fluorene, and the like.

When at least one of the aryl group, the fluorenyl group, the heterocyclic group, the aliphatic ring group, the arylene group and the fluorenylene group is substituted with an alkyl group, the alkyl group may be preferably a C₁-C₂₀, a C₁-C₁₀, a C₁-C₄, a C₁, a C₂, a C₃, or a C₄ alkyl group and the like.

When at least one of the aryl group, the fluorenyl group, the heterocyclic group, the aliphatic ring group, the arylene group and the fluorenylene group is substituted with an alkenyl group, the alkenyl group may be preferably a C₂-C₂₀, a C₂-C₁₀, a C₂-C₄, a C₂, a C₃, or a C₄ alkenyl group and the like.

Formula 1 can be represented by one of Formula 2 to Formula 1-8.

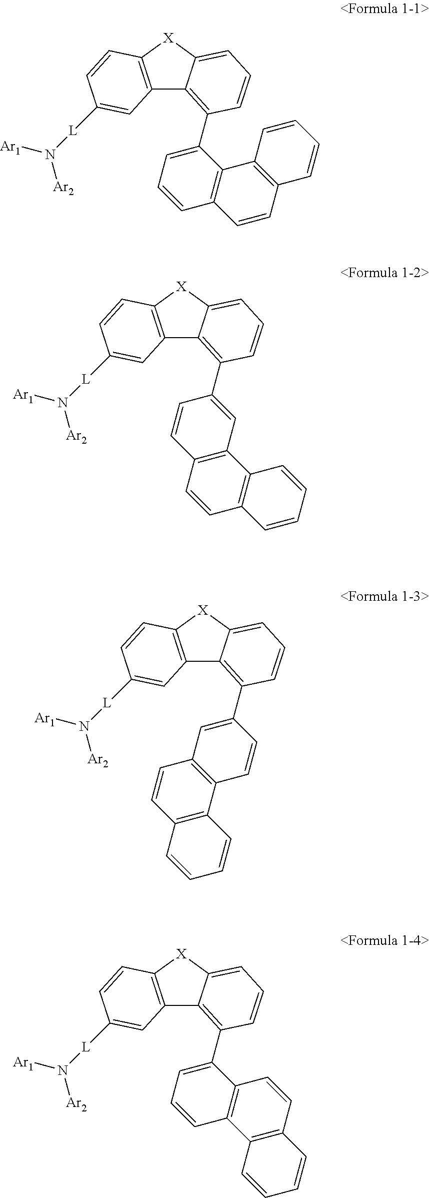

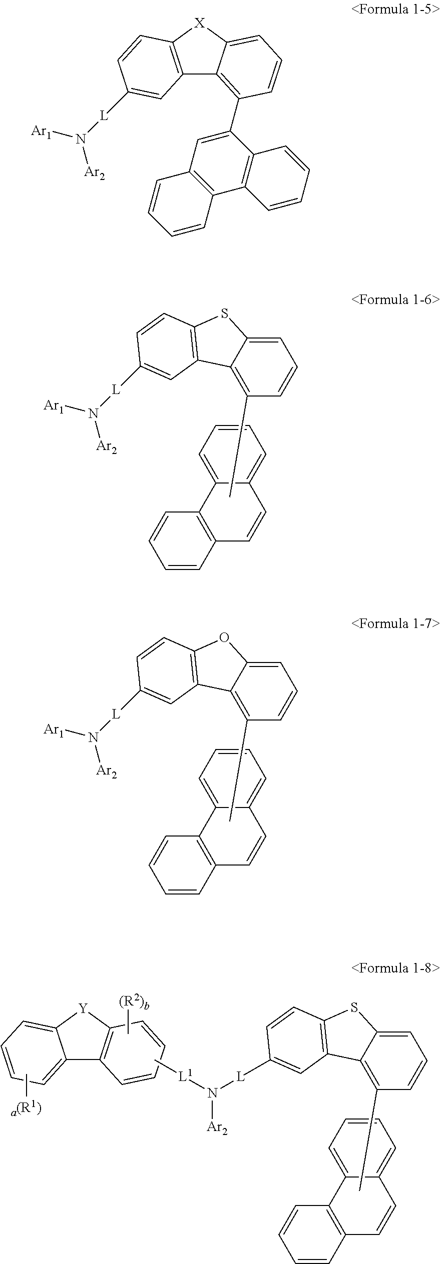

In Formula 1-1 to Formula 1-8, X, L, Ar₁ and Ar₂ are the same as defined for Formula 1.

In Formula 1-8, Y is O, S, C(R′)(R″) or N-(L₂-Ar₃).

R₁, R₂, R′ and R″ are each independently selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group containing at least one heteroatom of O, N, S, Si and P, and a C₃-C₃₀ aliphatic ring group, and adjacent groups may be bonded to each other to form a ring, and R′ and R″ may be bonded to each other to form a ring.

a is an integer of 0 to 4, b is an integer of 0 to 3, when each of these is an integer of 2 or more, a plurality of R¹s and a plurality of R²s are each the same as or different from each other, and adjacent R¹s, adjacent R²s may be bonded to each other to form a ring.

A ring formed by adjacent R¹s, adjacent R²s, R′ and R″ may be selected from the group consisting of a C₆-C₃₀ aromatic ring group, a fluorenyl group, a C₂-C₃₀ heterocyclic group containing at least one heteroatom of O, N, S, Si and P, and a C₃-C₃₀ aliphatic ring.

When an aromatic ring is formed by adjacent groups, the aromatic ring may be, for example, a C₆-C₂₀, a C₆-C₁₈, a C₆-C₁₆, a C₆-C₁₄, a C₆-C₁₃, a C₆-C₁₂, a C₆-C₁₀, a C₆, a C₁₀, a C₁₂, a C₁₄, a C₁₅, a C₁₆, or a C₁₈ aromatic ring, specifically, benzene, naphthalene, anthracene, phenanthrene, pyrene, etc.

R′ and R″ are bonded to each other to form a C₆-C₃₀ aromatic ring group, a fluorenyl group, a C₂-C₃₀ heterocyclic group containing at least one heteroatom of O, N, S, Si and P, or a C₃-C₃₀ aliphatic ring, for example, when R′ and R″ are bonded to each other to form a ring, a spiro-compound may be formed.

L¹ and L₂ are each independently selected from the group consisting of a single bond, a C₆-C₃₀ arylene group, a fluorenylene group, a C₂-C₃₀ heterocyclic group containing at least one heteroatom of O, N, S, Si and P, and a C₃-C₃₀ aliphatic ring group.

Ar₃ is selected from the group consisting of a C₆-C₃₀ aromatic ring group, a fluorenyl group, a C₂-C₃₀ heterocyclic group containing at least one heteroatom of O, N, S, Si and P, and a C₃-C₃₀ aliphatic ring.

When at least one of R¹, R², R′, R″ and Ar₃ is an aryl group, the aryl group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, a C₁₈, a C₁₉, a C₂₀, a C₂₁, a C₂₂, a C₂₃, a C₂₄, a C₂₅, a C₂₆, a C₂₇, a C₂₈, a C₂₉, or a C₃₀ aryl group.

When at least one of L¹ and L₂ is an arylene group, the arylene group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, a C₁₈, a C₁₉, a C₂₀, a C₂₁, a C₂₂, a C₂₃, a C₂₄, a C₂₅, a C₂₆, a C₂₇, a C₂₈, a C₂₉, or a C₃₀ arylene group.

When at least one of R¹, R², R′, R″, Ar₃, L¹ and L₂ is a heterocyclic group, the heterocyclic group may be, for example, a C₂-C₃₀, a C₂-C₂₉, a C₂-C₂₈, a C₂-C₂₇, a C₂-C₂₆, a C₂-C₂₅, a C₂-C₂₄, a C₂-C₂₃, a C₂-C₂₂, a C₂-C₂₁, a C₂-C₂₀, a C₂-C₁₉, a C₂-C₁₈, a C₂-C₁₇, a C₂-C₁₆, a C₂-C₁₅, a C₂-C₁₄, a C₂-C₁₃, a C₂-C₁₂, a C₂-C₁₁, a C₂-C₁₀, a C₂-C₉, a C₂-C₈, a C₂-C₇, a C₂-C₆, a C₂-C₅, a C₂-C₄, a C₂-C₃, a C₂, a C₃, a C₄, a C₅, a C₆, a C₇, a C₈, a C₉, a C₁₀, a C₁₁, a C₁₂, a C₁₃, a C₁₄, a C₁s, a C₁₆, a C₁₇, a C₁₈, a C₁₉, a C₂₀, a C₂₁, a C₂₂, a C₂₃, a C₂₄, a C₂s, a C₂₆, a C₂₇, a C₂₈, a C₂₉ or a C₃₀ heterocyclic group.

When at least one of R¹, R², R′ and R″ is an alkyl group, the alkyl group may be preferably a C₁-C₂₀, a C₁-C₁₀, a C₁-C₄, a C₁, a C₂, a C₃, or a C₄ alkyl group and the like, for example, a methyl group, ethyl group, t-butyl group, etc.

Specifically, the compound represented by Formula 1 may be one of the following compounds, but there is no limitation thereto.

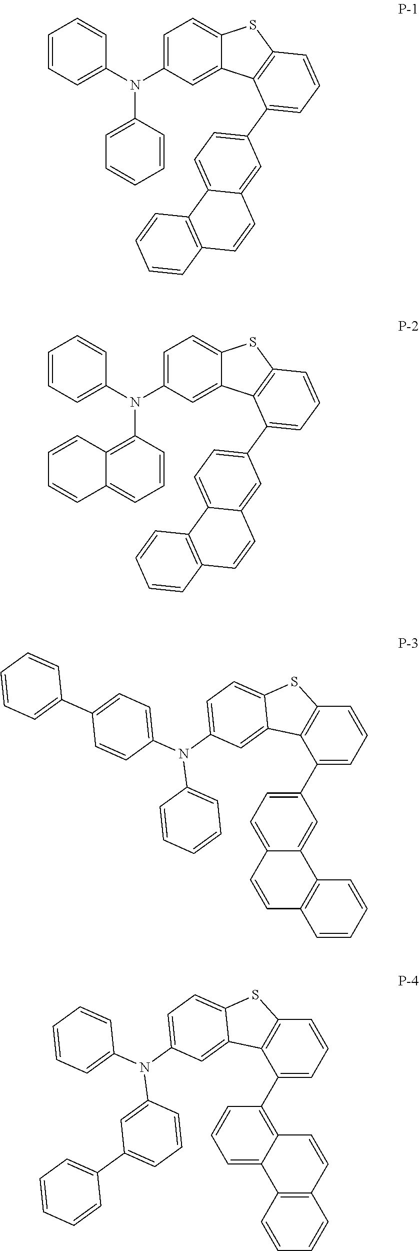 [SMILES: c1ccc(N(c2ccccc2)c2ccc3sc4cccc(-c5ccc6c(ccc7ccccc76)c5)c4c3c2)cc1 ; c1ccc(N(c2ccc3sc4cccc(-c5ccc6c(ccc7ccccc76)c5)c4c3c2)c2cccc3ccccc23)cc1 ; c1ccc(-c2ccc(N(c3ccccc3)c3ccc4sc5cccc(-c6ccc7ccc8ccccc8c7c6)c5c4c3)cc2)cc1 ; c1ccc(-c2cccc(N(c3ccccc3)c3ccc4sc5cccc(-c6cccc7c6ccc6ccccc67)c5c4c3)c2)cc1]

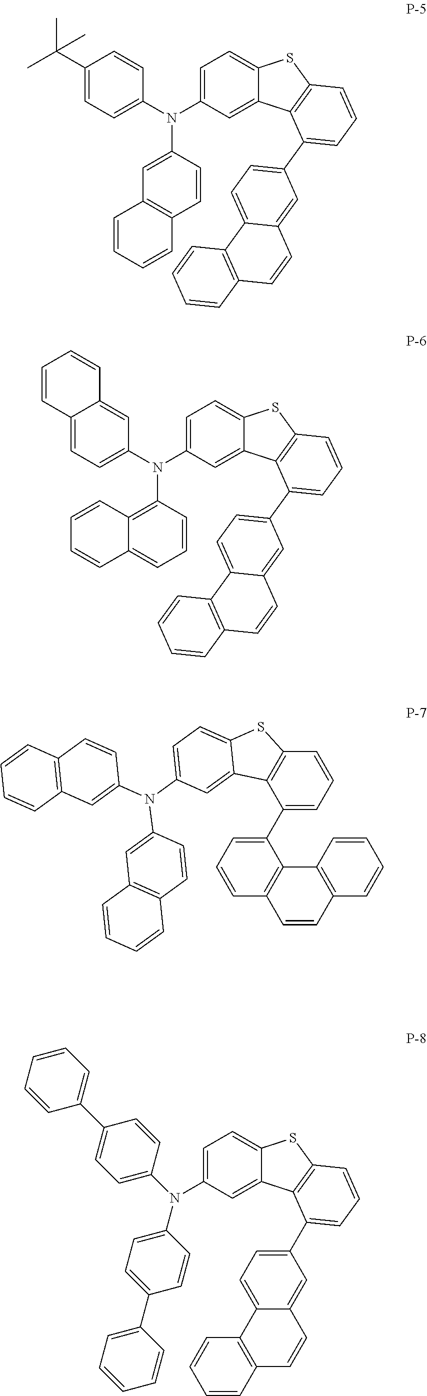 [SMILES: CC(C)(C)c1ccc(N(c2ccc3ccccc3c2)c2ccc3sc4cccc(-c5ccc6c(ccc7ccccc76)c5)c4c3c2)cc1 ; c1ccc2cc(N(c3ccc4sc5cccc(-c6ccc7c(ccc8ccccc87)c6)c5c4c3)c3cccc4ccccc34)ccc2c1 ; c1ccc2cc(N(c3ccc4ccccc4c3)c3ccc4sc5cccc(-c6cccc7ccc8ccccc8c67)c5c4c3)ccc2c1 ; c1ccc(-c2ccc(N(c3ccc(-c4ccccc4)cc3)c3ccc4sc5cccc(-c6ccc7c(ccc8ccccc87)c6)c5c4c3)cc2)cc1]

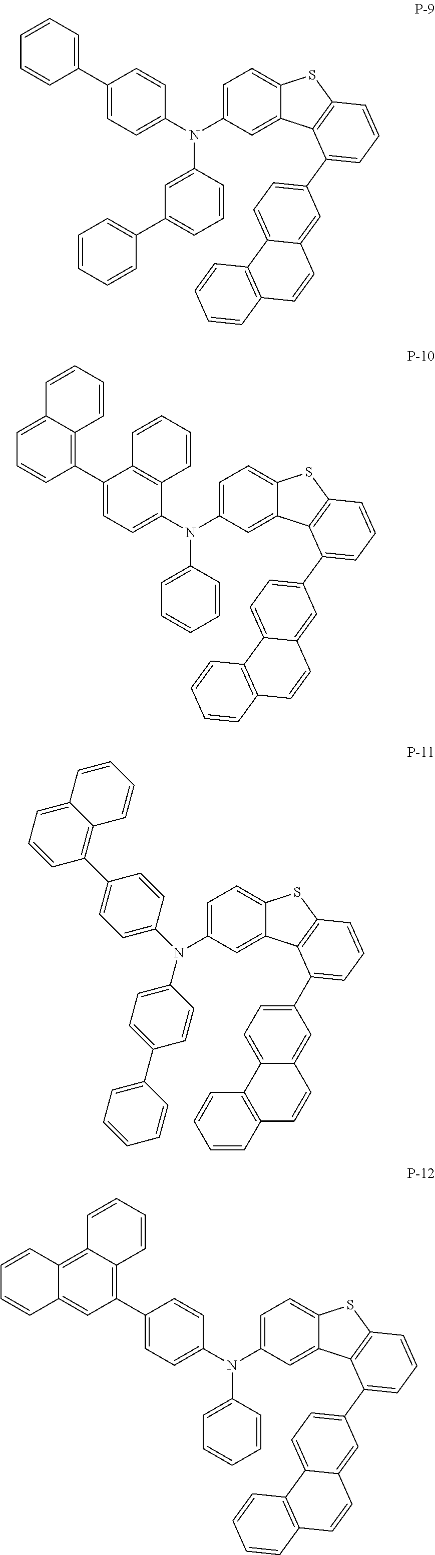 [SMILES: c1ccc(-c2ccc(N(c3cccc(-c4ccccc4)c3)c3ccc4sc5cccc(-c6ccc7c(ccc8ccccc87)c6)c5c4c3)cc2)cc1 ; c1ccc(N(c2ccc3sc4cccc(-c5ccc6c(ccc7ccccc76)c5)c4c3c2)c2ccc(-c3cccc4ccccc34)c3ccccc23)cc1 ; c1ccc(-c2ccc(N(c3ccc(-c4cccc5ccccc45)cc3)c3ccc4sc5cccc(-c6ccc7c(ccc8ccccc87)c6)c5c4c3)cc2)cc1 ; c1ccc(N(c2ccc(-c3cc4ccccc4c4ccccc34)cc2)c2ccc3sc4cccc(-c5ccc6c(ccc7ccccc76)c5)c4c3c2)cc1]

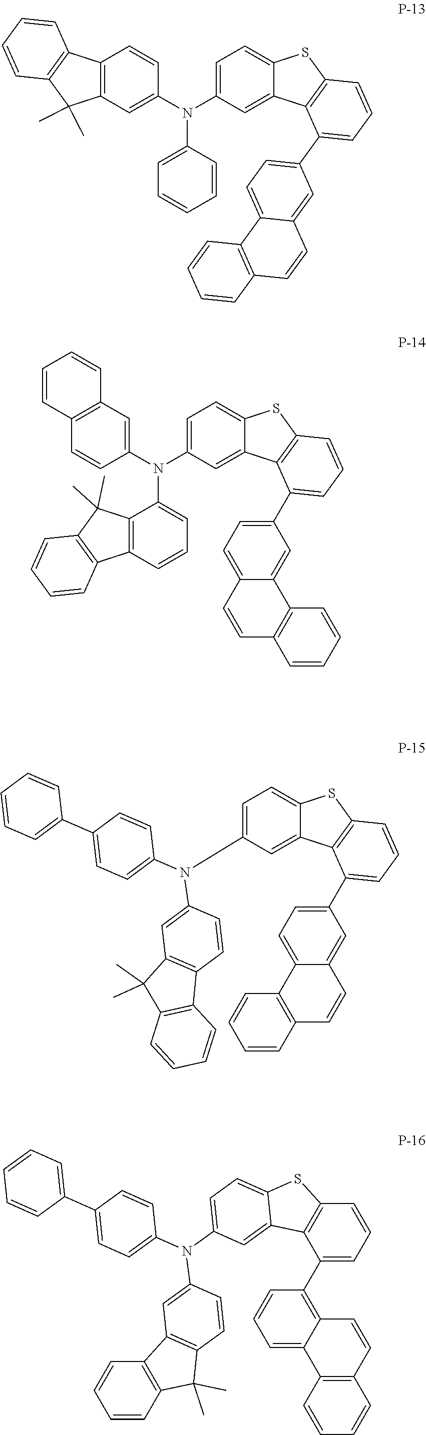 [SMILES: CC1(C)c2ccccc2-c2ccc(N(c3ccccc3)c3ccc4sc5cccc(-c6ccc7c(ccc8ccccc87)c6)c5c4c3)cc21 ; CC1(C)c2ccccc2-c2cccc(N(c3ccc4ccccc4c3)c3ccc4sc5cccc(-c6ccc7ccc8ccccc8c7c6)c5c4c3)c21 ; CC1(C)c2ccccc2-c2ccc(N(c3ccc(-c4ccccc4)cc3)c3ccc4sc5cccc(-c6ccc7c(ccc8ccccc87)c6)c5c4c3)cc21 ; CC1(C)c2ccccc2-c2cc(N(c3ccc(-c4ccccc4)cc3)c3ccc4sc5cccc(-c6cccc7c6ccc6ccccc67)c5c4c3)ccc21]

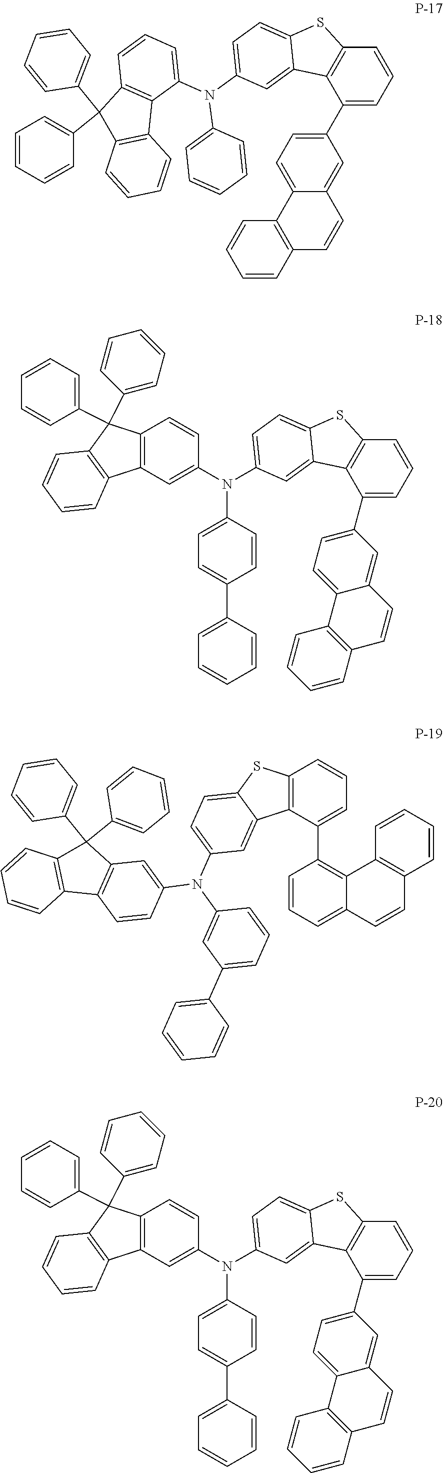 [SMILES: c1ccc(N(c2ccc3sc4cccc(-c5ccc6c(ccc7ccccc76)c5)c4c3c2)c2cccc3c2-c2ccccc2C3(c2ccccc2)c2ccccc2)cc1 ; c1ccc(-c2ccc(N(c3ccc4c(c3)-c3ccccc3C4(c3ccccc3)c3ccccc3)c3ccc4sc5cccc(-c6ccc7c(ccc8ccccc87)c6)c5c4c3)cc2)cc1 ; c1ccc(-c2cccc(N(c3ccc4c(c3)C(c3ccccc3)(c3ccccc3)c3ccccc3-4)c3ccc4sc5cccc(-c6cccc7ccc8ccccc8c67)c5c4c3)c2)cc1 ; c1ccc(-c2ccc(N(c3ccc4c(c3)-c3ccccc3C4(c3ccccc3)c3ccccc3)c3ccc4sc5cccc(-c6ccc7c(ccc8ccccc87)c6)c5c4c3)cc2)cc1]

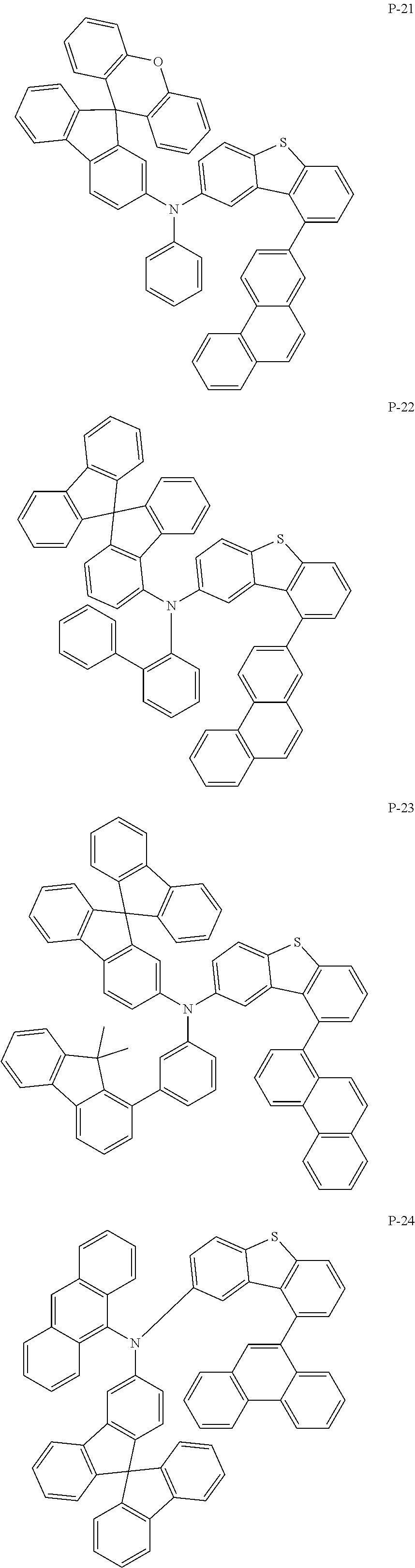 [SMILES: c1ccc(N(c2ccc3c(c2)C2(c4ccccc4Oc4ccccc42)c2ccccc2-3)c2ccc3sc4cccc(-c5ccc6c(ccc7ccccc76)c5)c4c3c2)cc1 ; c1ccc(-c2ccccc2N(c2ccc3sc4cccc(-c5ccc6c(ccc7ccccc76)c5)c4c3c2)c2cccc3c2-c2ccccc2C32c3ccccc3-c3ccccc32)cc1 ; CC1(C)c2ccccc2-c2cccc(-c3cccc(N(c4ccc5c(c4)C4(c6ccccc6-c6ccccc64)c4ccccc4-5)c4ccc5sc6cccc(-c7cccc8c7ccc7ccccc78)c6c5c4)c3)c21 ; c1ccc2c(c1)-c1ccccc1C21c2ccccc2-c2cc(N(c3ccc4sc5cccc(-c6cc7ccccc7c7ccccc67)c5c4c3)c3c4ccccc4cc4ccccc34)ccc21]

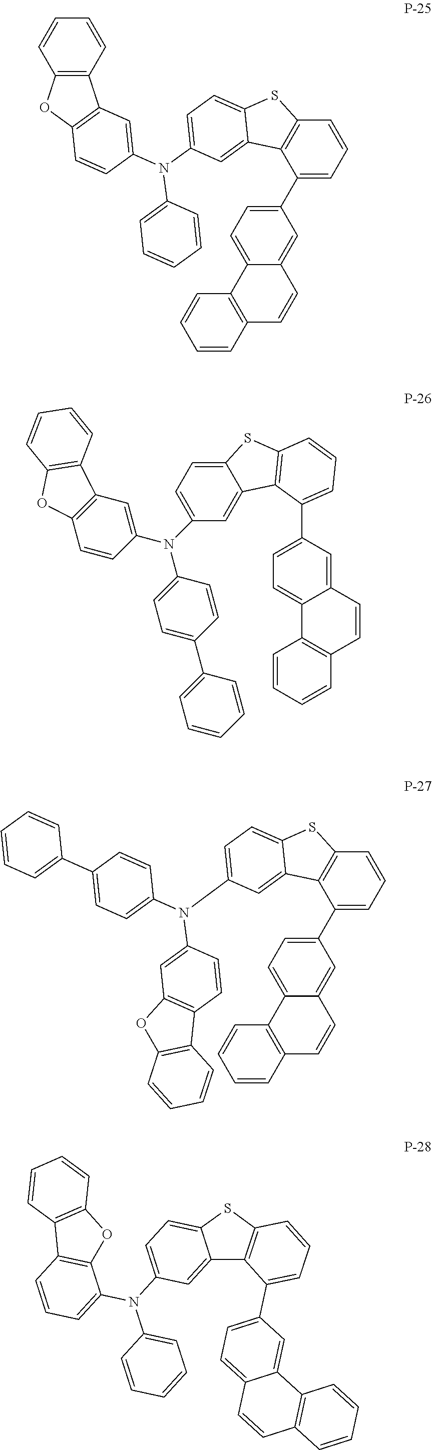 [SMILES: c1ccc(N(c2ccc3oc4ccccc4c3c2)c2ccc3sc4cccc(-c5ccc6c(ccc7ccccc76)c5)c4c3c2)cc1 ; c1ccc(-c2ccc(N(c3ccc4oc5ccccc5c4c3)c3ccc4sc5cccc(-c6ccc7c(ccc8ccccc87)c6)c5c4c3)cc2)cc1 ; c1ccc(-c2ccc(N(c3ccc4c(c3)oc3ccccc34)c3ccc4sc5cccc(-c6ccc7c(ccc8ccccc87)c6)c5c4c3)cc2)cc1 ; c1ccc(N(c2ccc3sc4cccc(-c5ccc6ccc7ccccc7c6c5)c4c3c2)c2cccc3c2oc2ccccc23)cc1]

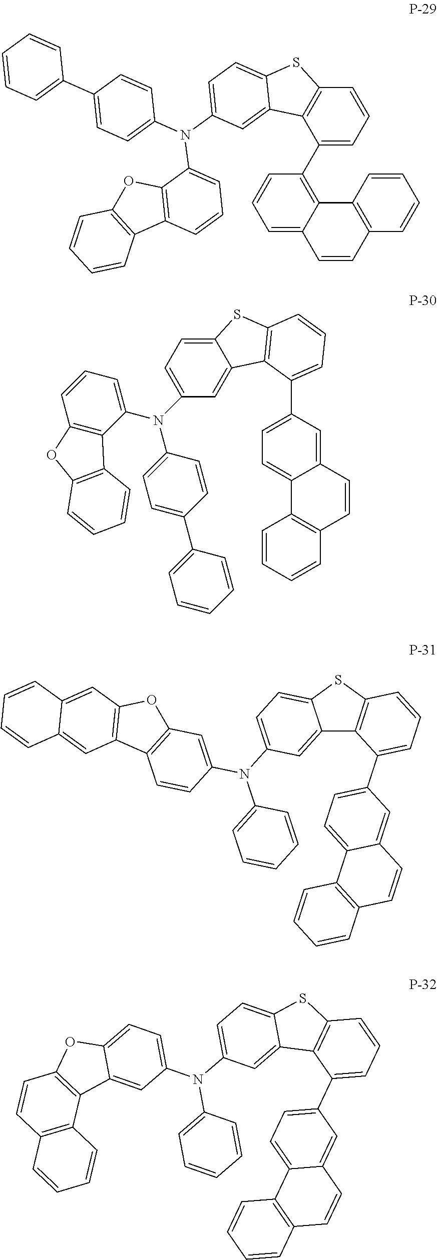 [SMILES: c1ccc(-c2ccc(N(c3ccc4sc5cccc(-c6cccc7ccc8ccccc8c67)c5c4c3)c3cccc4c3oc3ccccc34)cc2)cc1 ; c1ccc(-c2ccc(N(c3ccc4sc5cccc(-c6ccc7c(ccc8ccccc87)c6)c5c4c3)c3cccc4oc5ccccc5c34)cc2)cc1 ; c1ccc(N(c2ccc3c(c2)oc2cc4ccccc4cc23)c2ccc3sc4cccc(-c5ccc6c(ccc7ccccc76)c5)c4c3c2)cc1 ; c1ccc(N(c2ccc3sc4cccc(-c5ccc6c(ccc7ccccc76)c5)c4c3c2)c2ccc3oc4ccc5ccccc5c4c3c2)cc1]

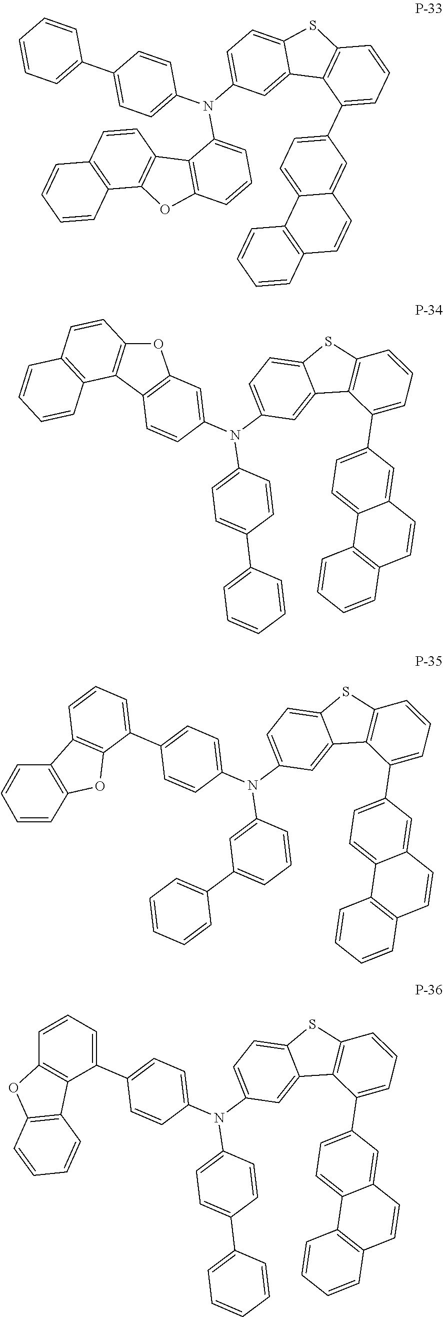 [SMILES: c1ccc(-c2ccc(N(c3ccc4sc5cccc(-c6ccc7c(ccc8ccccc87)c6)c5c4c3)c3cccc4oc5c6ccccc6ccc5c34)cc2)cc1 ; c1ccc(-c2ccc(N(c3ccc4c(c3)oc3ccc5ccccc5c34)c3ccc4sc5cccc(-c6ccc7c(ccc8ccccc87)c6)c5c4c3)cc2)cc1 ; c1ccc(-c2cccc(N(c3ccc(-c4cccc5c4oc4ccccc45)cc3)c3ccc4sc5cccc(-c6ccc7c(ccc8ccccc87)c6)c5c4c3)c2)cc1 ; c1ccc(-c2ccc(N(c3ccc(-c4cccc5oc6ccccc6c45)cc3)c3ccc4sc5cccc(-c6ccc7c(ccc8ccccc87)c6)c5c4c3)cc2)cc1]

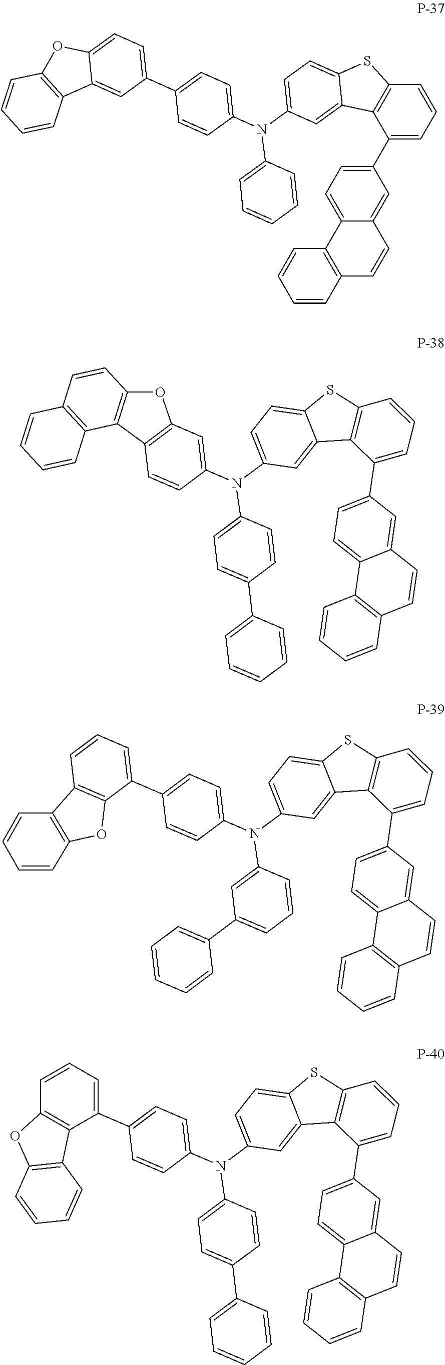 [SMILES: c1ccc(N(c2ccc(-c3ccc4oc5ccccc5c4c3)cc2)c2ccc3sc4cccc(-c5ccc6c(ccc7ccccc76)c5)c4c3c2)cc1 ; c1ccc(-c2ccc(N(c3ccc4c(c3)oc3ccc5ccccc5c34)c3ccc4sc5cccc(-c6ccc7c(ccc8ccccc87)c6)c5c4c3)cc2)cc1 ; c1ccc(-c2cccc(N(c3ccc(-c4cccc5c4oc4ccccc45)cc3)c3ccc4sc5cccc(-c6ccc7c(ccc8ccccc87)c6)c5c4c3)c2)cc1 ; c1ccc(-c2ccc(N(c3ccc(-c4cccc5oc6ccccc6c45)cc3)c3ccc4sc5cccc(-c6ccc7c(ccc8ccccc87)c6)c5c4c3)cc2)cc1]

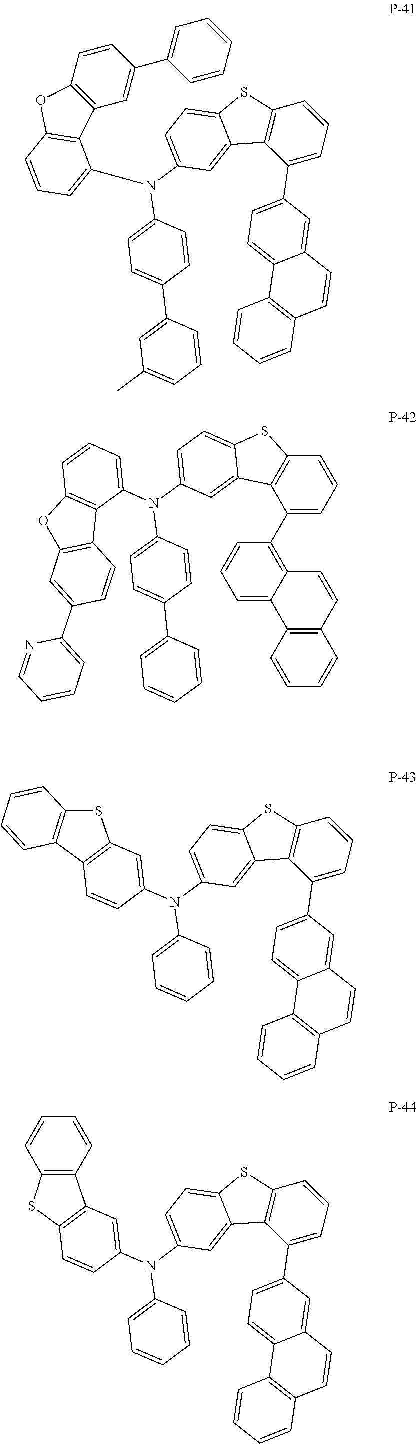 [SMILES: Cc1cccc(-c2ccc(N(c3ccc4sc5cccc(-c6ccc7c(ccc8ccccc87)c6)c5c4c3)c3cccc4oc5ccc(-c6ccccc6)cc5c34)cc2)c1 ; c1ccc(-c2ccc(N(c3ccc4sc5cccc(-c6cccc7c6ccc6ccccc67)c5c4c3)c3cccc4oc5cc(-c6ccccn6)ccc5c34)cc2)cc1 ; c1ccc(N(c2ccc3c(c2)sc2ccccc23)c2ccc3sc4cccc(-c5ccc6c(ccc7ccccc76)c5)c4c3c2)cc1 ; c1ccc(N(c2ccc3sc4ccccc4c3c2)c2ccc3sc4cccc(-c5ccc6c(ccc7ccccc76)c5)c4c3c2)cc1]

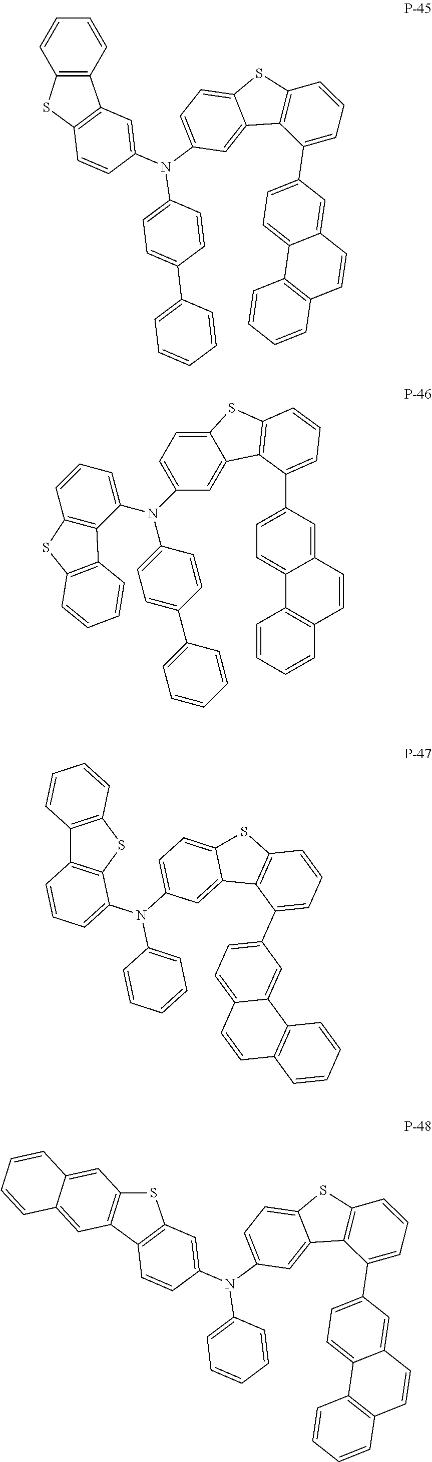 [SMILES: c1ccc(-c2ccc(N(c3ccc4sc5ccccc5c4c3)c3ccc4sc5cccc(-c6ccc7c(ccc8ccccc87)c6)c5c4c3)cc2)cc1 ; c1ccc(-c2ccc(N(c3ccc4sc5cccc(-c6ccc7c(ccc8ccccc87)c6)c5c4c3)c3cccc4sc5ccccc5c34)cc2)cc1 ; c1ccc(N(c2ccc3sc4cccc(-c5ccc6ccc7ccccc7c6c5)c4c3c2)c2cccc3c2sc2ccccc23)cc1 ; c1ccc(N(c2ccc3c(c2)sc2cc4ccccc4cc23)c2ccc3sc4cccc(-c5ccc6c(ccc7ccccc76)c5)c4c3c2)cc1]

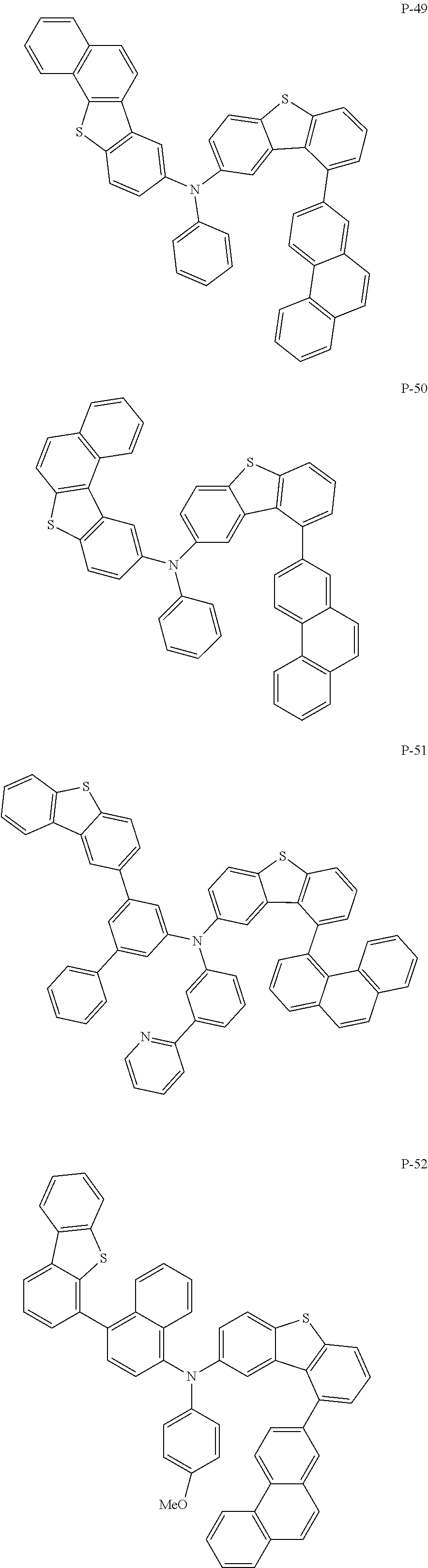 [SMILES: c1ccc(N(c2ccc3sc4c5ccccc5ccc4c3c2)c2ccc3sc4cccc(-c5ccc6c(ccc7ccccc76)c5)c4c3c2)cc1 ; c1ccc(N(c2ccc3sc4cccc(-c5ccc6c(ccc7ccccc76)c5)c4c3c2)c2ccc3sc4ccc5ccccc5c4c3c2)cc1 ; c1ccc(-c2cc(-c3ccc4sc5ccccc5c4c3)cc(N(c3cccc(-c4ccccn4)c3)c3ccc4sc5cccc(-c6cccc7ccc8ccccc8c67)c5c4c3)c2)cc1 ; COc1ccc(N(c2ccc3sc4cccc(-c5ccc6c(ccc7ccccc76)c5)c4c3c2)c2ccc(-c3cccc4c3sc3ccccc34)c3ccccc23)cc1]

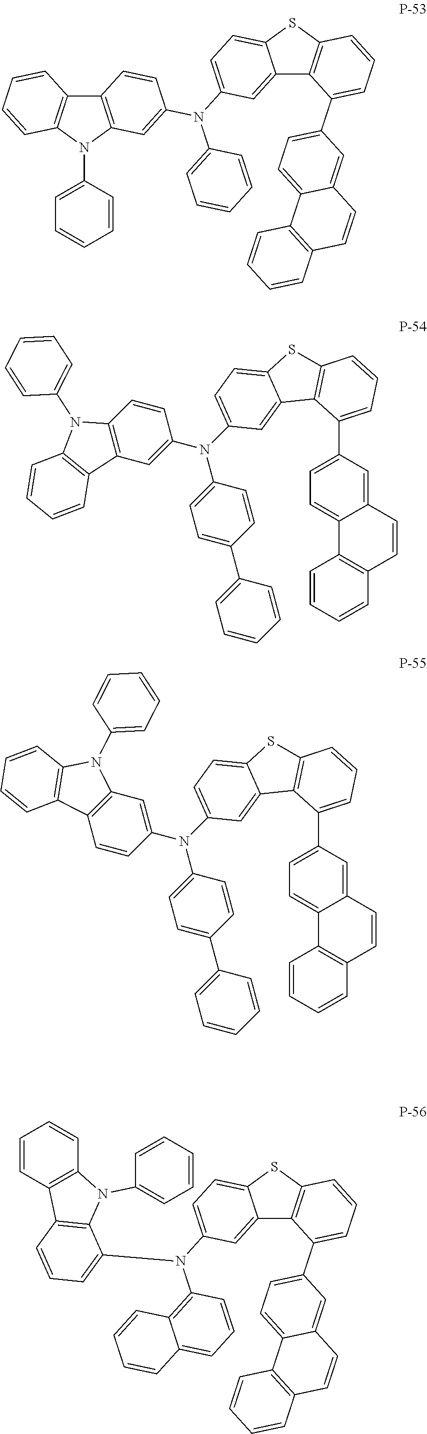 [SMILES: c1ccc(N(c2ccc3sc4cccc(-c5ccc6c(ccc7ccccc76)c5)c4c3c2)c2ccc3c4ccccc4n(-c4ccccc4)c3c2)cc1 ; c1ccc(-c2ccc(N(c3ccc4sc5cccc(-c6ccc7c(ccc8ccccc87)c6)c5c4c3)c3ccc4c(c3)c3ccccc3n4-c3ccccc3)cc2)cc1 ; c1ccc(-c2ccc(N(c3ccc4sc5cccc(-c6ccc7c(ccc8ccccc87)c6)c5c4c3)c3ccc4c5ccccc5n(-c5ccccc5)c4c3)cc2)cc1 ; c1ccc(-n2c3ccccc3c3cccc(N(c4ccc5sc6cccc(-c7ccc8c(ccc9ccccc98)c7)c6c5c4)c4cccc5ccccc45)c32)cc1]

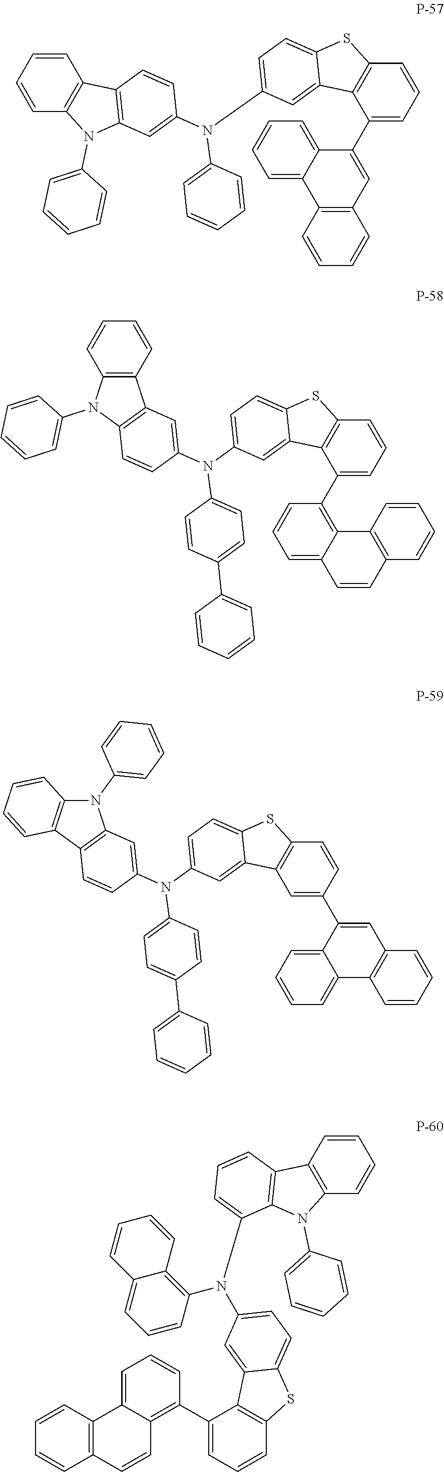 [SMILES: c1ccc(N(c2ccc3sc4cccc(-c5cc6ccccc6c6ccccc56)c4c3c2)c2ccc3c4ccccc4n(-c4ccccc4)c3c2)cc1 ; c1ccc(-c2ccc(N(c3ccc4sc5cccc(-c6cccc7ccc8ccccc8c67)c5c4c3)c3ccc4c(c3)c3ccccc3n4-c3ccccc3)cc2)cc1 ; c1ccc(-c2ccc(N(c3ccc4sc5ccc(-c6cc7ccccc7c7ccccc67)cc5c4c3)c3ccc4c5ccccc5n(-c5ccccc5)c4c3)cc2)cc1 ; c1ccc(-n2c3ccccc3c3cccc(N(c4ccc5sc6cccc(-c7cccc8c7ccc7ccccc78)c6c5c4)c4cccc5ccccc45)c32)cc1]

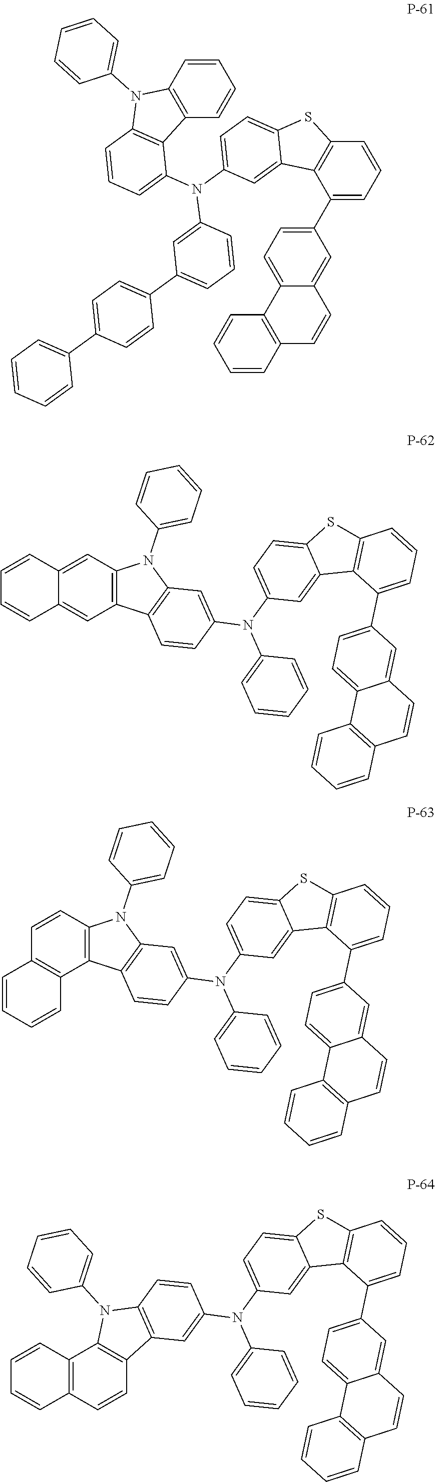 [SMILES: c1ccc(-c2ccc(-c3cccc(N(c4ccc5sc6cccc(-c7ccc8c(ccc9ccccc98)c7)c6c5c4)c4cccc5c4c4ccccc4n5-c4ccccc4)c3)cc2)cc1 ; c1ccc(N(c2ccc3sc4cccc(-c5ccc6c(ccc7ccccc76)c5)c4c3c2)c2ccc3c4cc5ccccc5cc4n(-c4ccccc4)c3c2)cc1 ; c1ccc(N(c2ccc3sc4cccc(-c5ccc6c(ccc7ccccc76)c5)c4c3c2)c2ccc3c4c5ccccc5ccc4n(-c4ccccc4)c3c2)cc1 ; c1ccc(N(c2ccc3sc4cccc(-c5ccc6c(ccc7ccccc76)c5)c4c3c2)c2ccc3c(c2)c2ccc4ccccc4c2n3-c2ccccc2)cc1]

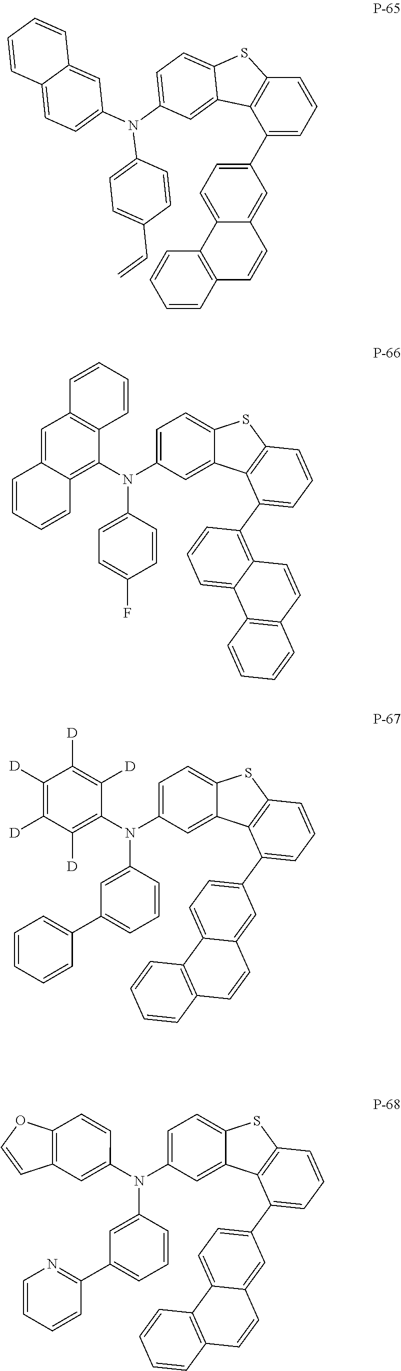 [SMILES: C=Cc1ccc(N(c2ccc3ccccc3c2)c2ccc3sc4cccc(-c5ccc6c(ccc7ccccc76)c5)c4c3c2)cc1 ; Fc1ccc(N(c2ccc3sc4cccc(-c5cccc6c5ccc5ccccc56)c4c3c2)c2c3ccccc3cc3ccccc23)cc1 ; [2H]c1c([2H])c([2H])c(N(c2cccc(-c3ccccc3)c2)c2ccc3sc4cccc(-c5ccc6c(ccc7ccccc76)c5)c4c3c2)c([2H])c1[2H] ; c1ccc(-c2cccc(N(c3ccc4occc4c3)c3ccc4sc5cccc(-c6ccc7c(ccc8ccccc87)c6)c5c4c3)c2)nc1]

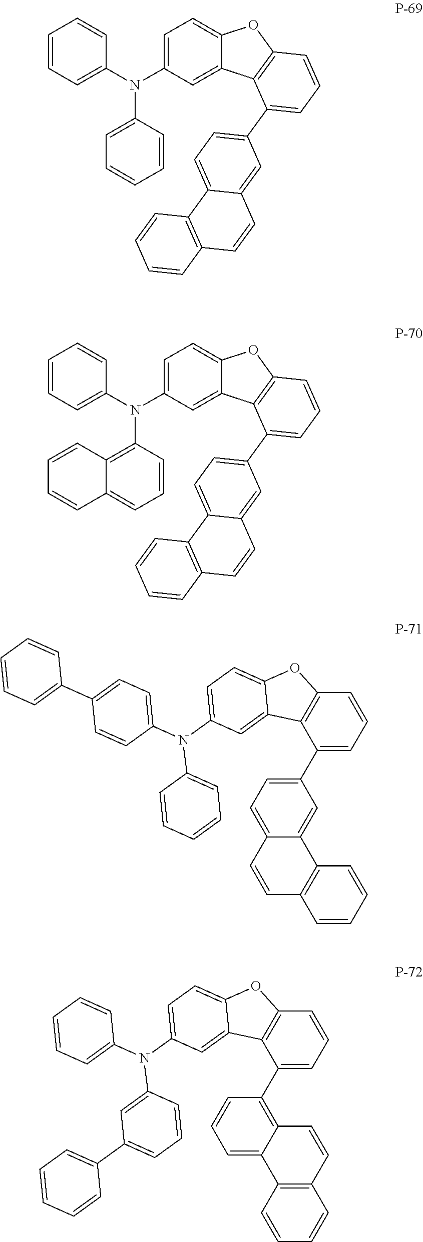 [SMILES: c1ccc(N(c2ccccc2)c2ccc3oc4cccc(-c5ccc6c(ccc7ccccc76)c5)c4c3c2)cc1 ; c1ccc(N(c2ccc3oc4cccc(-c5ccc6c(ccc7ccccc76)c5)c4c3c2)c2cccc3ccccc23)cc1 ; c1ccc(-c2ccc(N(c3ccccc3)c3ccc4oc5cccc(-c6ccc7ccc8ccccc8c7c6)c5c4c3)cc2)cc1 ; c1ccc(-c2cccc(N(c3ccccc3)c3ccc4oc5cccc(-c6cccc7c6ccc6ccccc67)c5c4c3)c2)cc1]

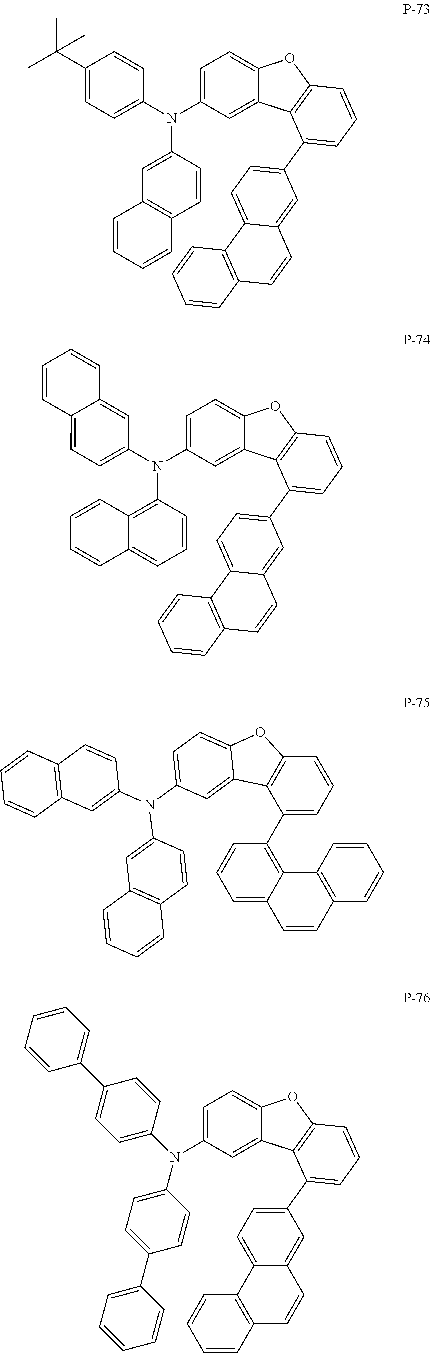 [SMILES: CC(C)(C)c1ccc(N(c2ccc3ccccc3c2)c2ccc3oc4cccc(-c5ccc6c(ccc7ccccc76)c5)c4c3c2)cc1 ; c1ccc2cc(N(c3ccc4oc5cccc(-c6ccc7c(ccc8ccccc87)c6)c5c4c3)c3cccc4ccccc34)ccc2c1 ; c1ccc2cc(N(c3ccc4ccccc4c3)c3ccc4oc5cccc(-c6cccc7ccc8ccccc8c67)c5c4c3)ccc2c1 ; c1ccc(-c2ccc(N(c3ccc(-c4ccccc4)cc3)c3ccc4oc5cccc(-c6ccc7c(ccc8ccccc87)c6)c5c4c3)cc2)cc1]

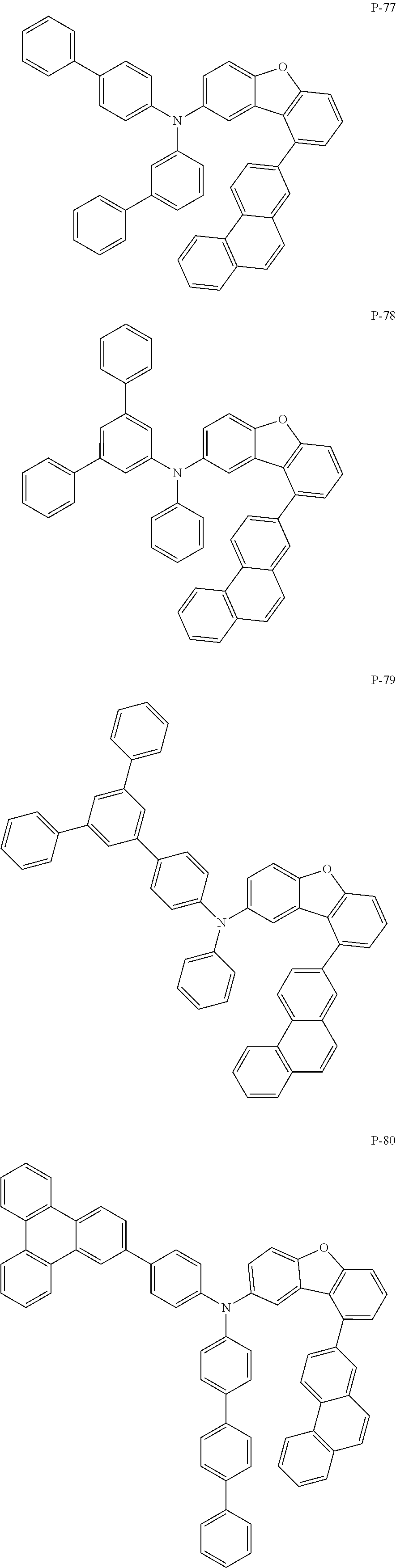 [SMILES: c1ccc(-c2ccc(N(c3cccc(-c4ccccc4)c3)c3ccc4oc5cccc(-c6ccc7c(ccc8ccccc87)c6)c5c4c3)cc2)cc1 ; c1ccc(-c2cc(-c3ccccc3)cc(N(c3ccccc3)c3ccc4oc5cccc(-c6ccc7c(ccc8ccccc87)c6)c5c4c3)c2)cc1 ; c1ccc(-c2cc(-c3ccccc3)cc(-c3ccc(N(c4ccccc4)c4ccc5oc6cccc(-c7ccc8c(ccc9ccccc98)c7)c6c5c4)cc3)c2)cc1 ; c1ccc(-c2ccc(-c3ccc(N(c4ccc(-c5ccc6c7ccccc7c7ccccc7c6c5)cc4)c4ccc5oc6cccc(-c7ccc8c(ccc9ccccc98)c7)c6c5c4)cc3)cc2)cc1]

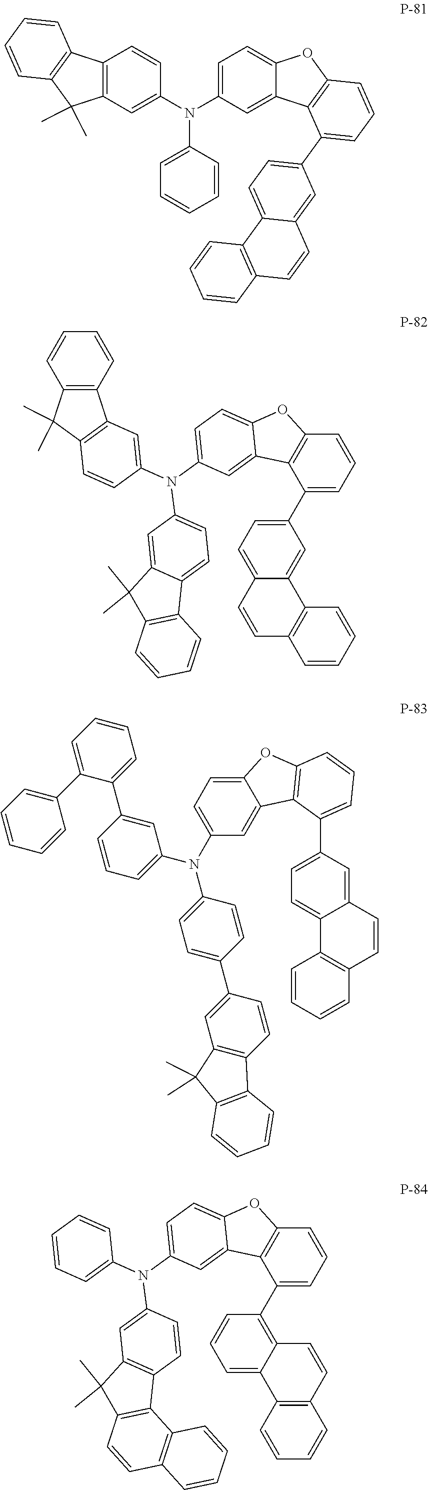 [SMILES: CC1(C)c2ccccc2-c2ccc(N(c3ccccc3)c3ccc4oc5cccc(-c6ccc7c(ccc8ccccc87)c6)c5c4c3)cc21 ; CC1(C)c2ccccc2-c2cc(N(c3ccc4c(c3)C(C)(C)c3ccccc3-4)c3ccc4oc5cccc(-c6ccc7ccc8ccccc8c7c6)c5c4c3)ccc21 ; CC1(C)c2ccccc2-c2ccc(-c3ccc(N(c4cccc(-c5ccccc5-c5ccccc5)c4)c4ccc5oc6cccc(-c7ccc8c(ccc9ccccc98)c7)c6c5c4)cc3)cc21 ; CC1(C)c2cc(N(c3ccccc3)c3ccc4oc5cccc(-c6cccc7c6ccc6ccccc67)c5c4c3)ccc2-c2c1ccc1ccccc21]

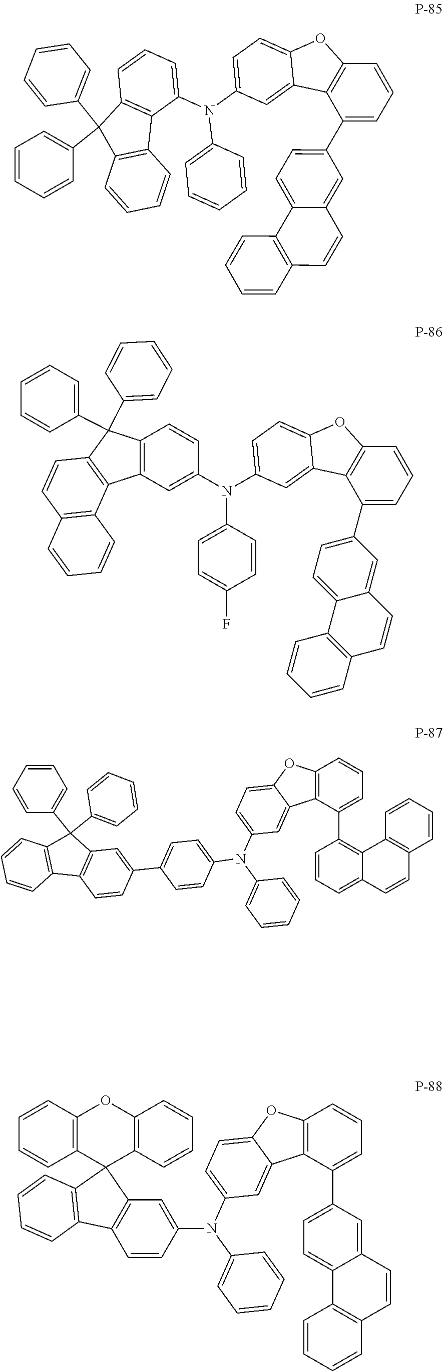 [SMILES: c1ccc(N(c2ccc3oc4cccc(-c5ccc6c(ccc7ccccc76)c5)c4c3c2)c2cccc3c2-c2ccccc2C3(c2ccccc2)c2ccccc2)cc1 ; Fc1ccc(N(c2ccc3c(c2)-c2c(ccc4ccccc24)C3(c2ccccc2)c2ccccc2)c2ccc3oc4cccc(-c5ccc6c(ccc7ccccc76)c5)c4c3c2)cc1 ; c1ccc(N(c2ccc(-c3ccc4c(c3)C(c3ccccc3)(c3ccccc3)c3ccccc3-4)cc2)c2ccc3oc4cccc(-c5cccc6ccc7ccccc7c56)c4c3c2)cc1 ; c1ccc(N(c2ccc3c(c2)C2(c4ccccc4Oc4ccccc42)c2ccccc2-3)c2ccc3oc4cccc(-c5ccc6c(ccc7ccccc76)c5)c4c3c2)cc1]

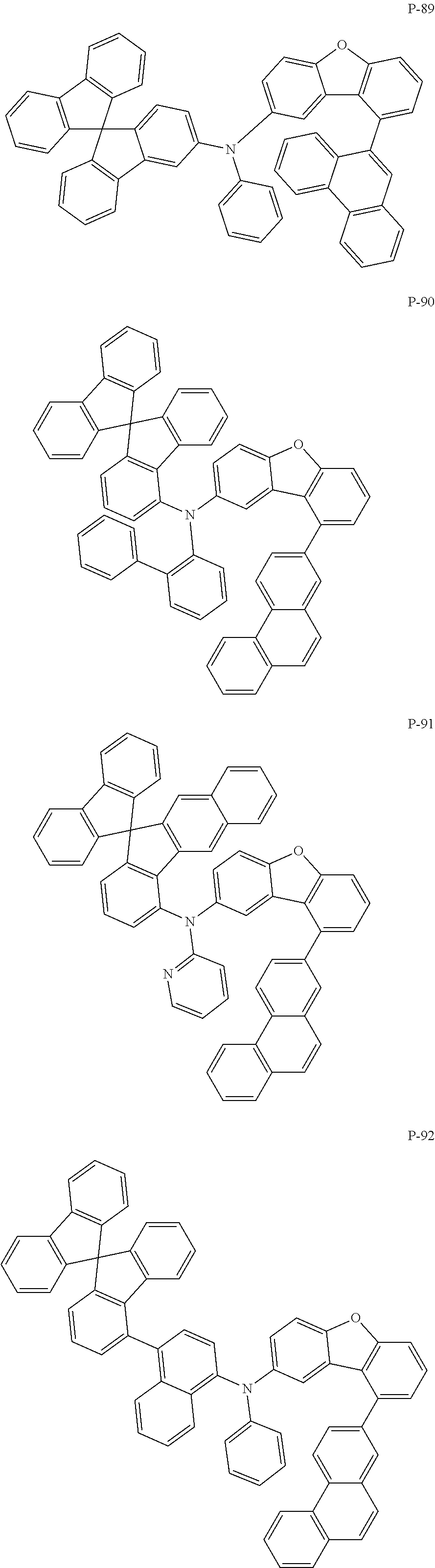 [SMILES: c1ccc(N(c2ccc3c(c2)-c2ccccc2C32c3ccccc3-c3ccccc32)c2ccc3oc4cccc(-c5cc6ccccc6c6ccccc56)c4c3c2)cc1 ; c1ccc(-c2ccccc2N(c2ccc3oc4cccc(-c5ccc6c(ccc7ccccc76)c5)c4c3c2)c2cccc3c2-c2ccccc2C32c3ccccc3-c3ccccc32)cc1 ; c1ccc(N(c2ccc3oc4cccc(-c5ccc6c(ccc7ccccc76)c5)c4c3c2)c2cccc3c2-c2cc4ccccc4cc2C32c3ccccc3-c3ccccc32)nc1 ; c1ccc(N(c2ccc3oc4cccc(-c5ccc6c(ccc7ccccc76)c5)c4c3c2)c2ccc(-c3cccc4c3-c3ccccc3C43c4ccccc4-c4ccccc43)c3ccccc23)cc1]

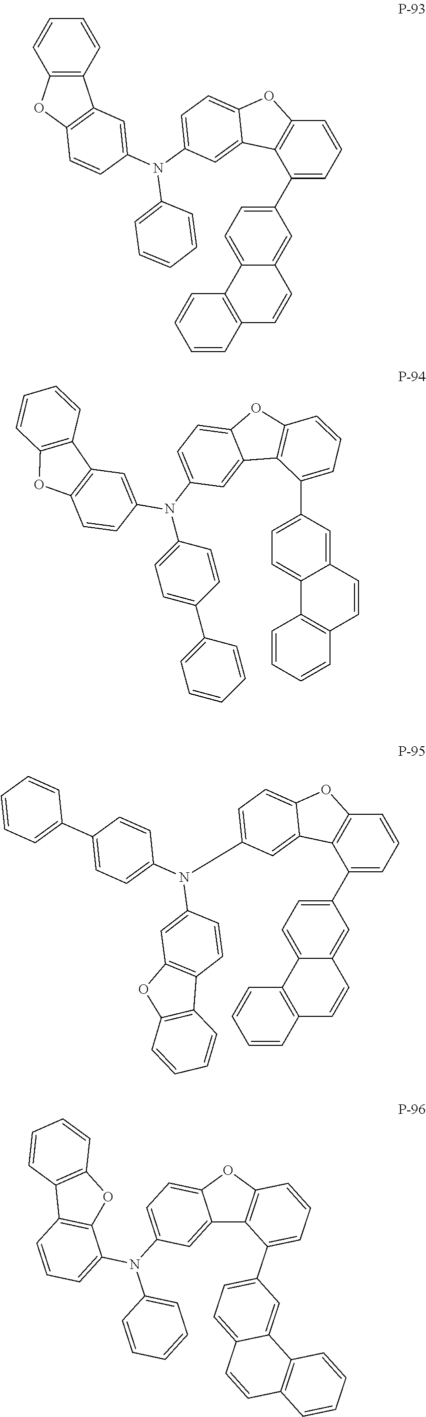 [SMILES: c1ccc(N(c2ccc3oc4ccccc4c3c2)c2ccc3oc4cccc(-c5ccc6c(ccc7ccccc76)c5)c4c3c2)cc1 ; c1ccc(-c2ccc(N(c3ccc4oc5ccccc5c4c3)c3ccc4oc5cccc(-c6ccc7c(ccc8ccccc87)c6)c5c4c3)cc2)cc1 ; c1ccc(-c2ccc(N(c3ccc4c(c3)oc3ccccc34)c3ccc4oc5cccc(-c6ccc7c(ccc8ccccc87)c6)c5c4c3)cc2)cc1 ; c1ccc(N(c2ccc3oc4cccc(-c5ccc6ccc7ccccc7c6c5)c4c3c2)c2cccc3c2oc2ccccc23)cc1]

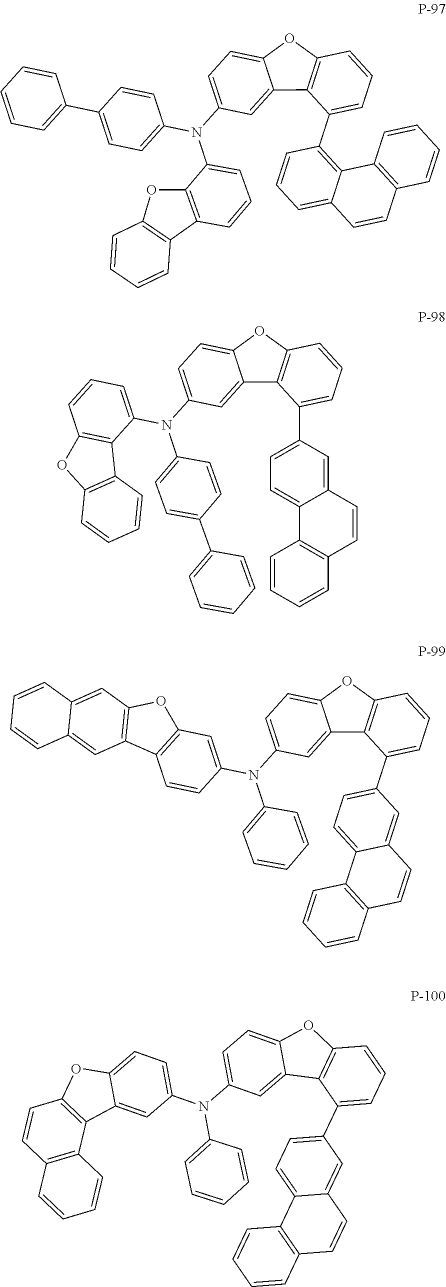 [SMILES: c1ccc(-c2ccc(N(c3ccc4oc5cccc(-c6cccc7ccc8ccccc8c67)c5c4c3)c3cccc4c3oc3ccccc34)cc2)cc1 ; c1ccc(-c2ccc(N(c3ccc4oc5cccc(-c6ccc7c(ccc8ccccc87)c6)c5c4c3)c3cccc4oc5ccccc5c34)cc2)cc1 ; c1ccc(N(c2ccc3c(c2)oc2cc4ccccc4cc23)c2ccc3oc4cccc(-c5ccc6c(ccc7ccccc76)c5)c4c3c2)cc1 ; c1ccc(N(c2ccc3oc4cccc(-c5ccc6c(ccc7ccccc76)c5)c4c3c2)c2ccc3oc4ccc5ccccc5c4c3c2)cc1]

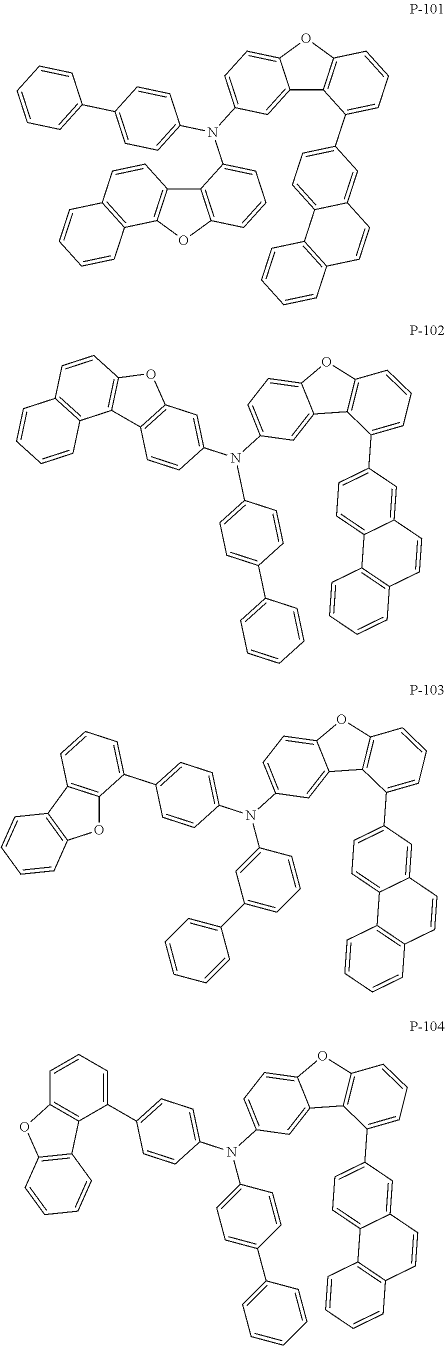 [SMILES: c1ccc(-c2ccc(N(c3ccc4oc5cccc(-c6ccc7c(ccc8ccccc87)c6)c5c4c3)c3cccc4oc5c6ccccc6ccc5c34)cc2)cc1 ; c1ccc(-c2ccc(N(c3ccc4c(c3)oc3ccc5ccccc5c34)c3ccc4oc5cccc(-c6ccc7c(ccc8ccccc87)c6)c5c4c3)cc2)cc1 ; c1ccc(-c2cccc(N(c3ccc(-c4cccc5c4oc4ccccc45)cc3)c3ccc4oc5cccc(-c6ccc7c(ccc8ccccc87)c6)c5c4c3)c2)cc1 ; c1ccc(-c2ccc(N(c3ccc(-c4cccc5oc6ccccc6c45)cc3)c3ccc4oc5cccc(-c6ccc7c(ccc8ccccc87)c6)c5c4c3)cc2)cc1]

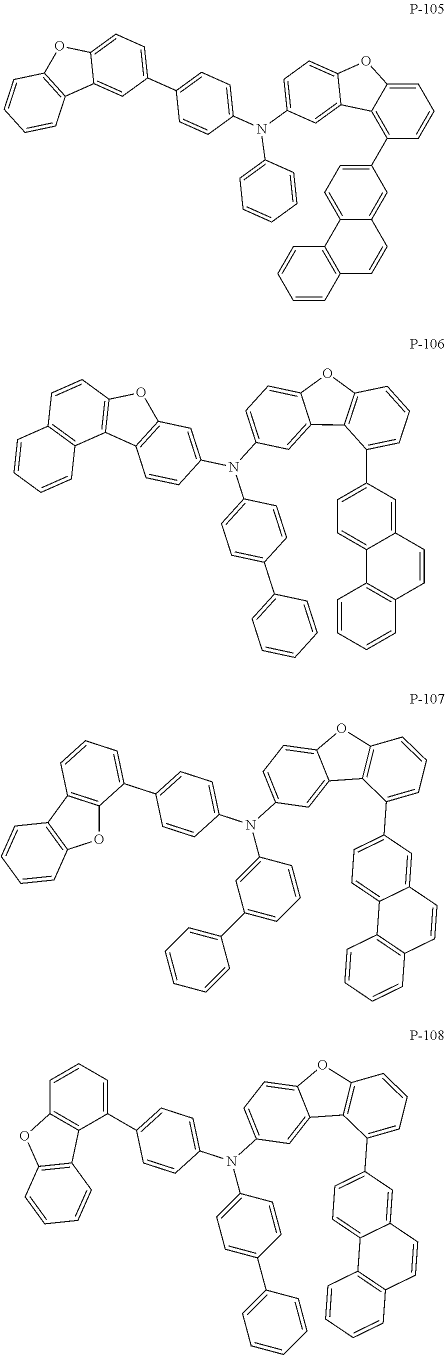 [SMILES: c1ccc(N(c2ccc(-c3ccc4oc5ccccc5c4c3)cc2)c2ccc3oc4cccc(-c5ccc6c(ccc7ccccc76)c5)c4c3c2)cc1 ; c1ccc(-c2ccc(N(c3ccc4c(c3)oc3ccc5ccccc5c34)c3ccc4oc5cccc(-c6ccc7c(ccc8ccccc87)c6)c5c4c3)cc2)cc1 ; c1ccc(-c2cccc(N(c3ccc(-c4cccc5c4oc4ccccc45)cc3)c3ccc4oc5cccc(-c6ccc7c(ccc8ccccc87)c6)c5c4c3)c2)cc1 ; c1ccc(-c2ccc(N(c3ccc(-c4cccc5oc6ccccc6c45)cc3)c3ccc4oc5cccc(-c6ccc7c(ccc8ccccc87)c6)c5c4c3)cc2)cc1]

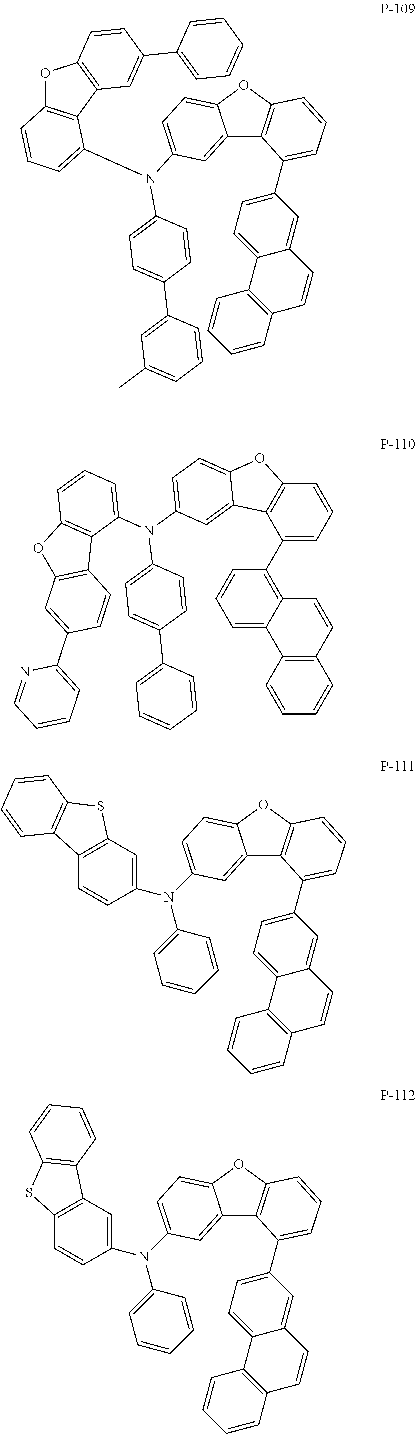 [SMILES: Cc1cccc(-c2ccc(N(c3ccc4oc5cccc(-c6ccc7c(ccc8ccccc87)c6)c5c4c3)c3cccc4oc5ccc(-c6ccccc6)cc5c34)cc2)c1 ; c1ccc(-c2ccc(N(c3ccc4oc5cccc(-c6cccc7c6ccc6ccccc67)c5c4c3)c3cccc4oc5cc(-c6ccccn6)ccc5c34)cc2)cc1 ; c1ccc(N(c2ccc3c(c2)sc2ccccc23)c2ccc3oc4cccc(-c5ccc6c(ccc7ccccc76)c5)c4c3c2)cc1 ; c1ccc(N(c2ccc3sc4ccccc4c3c2)c2ccc3oc4cccc(-c5ccc6c(ccc7ccccc76)c5)c4c3c2)cc1]

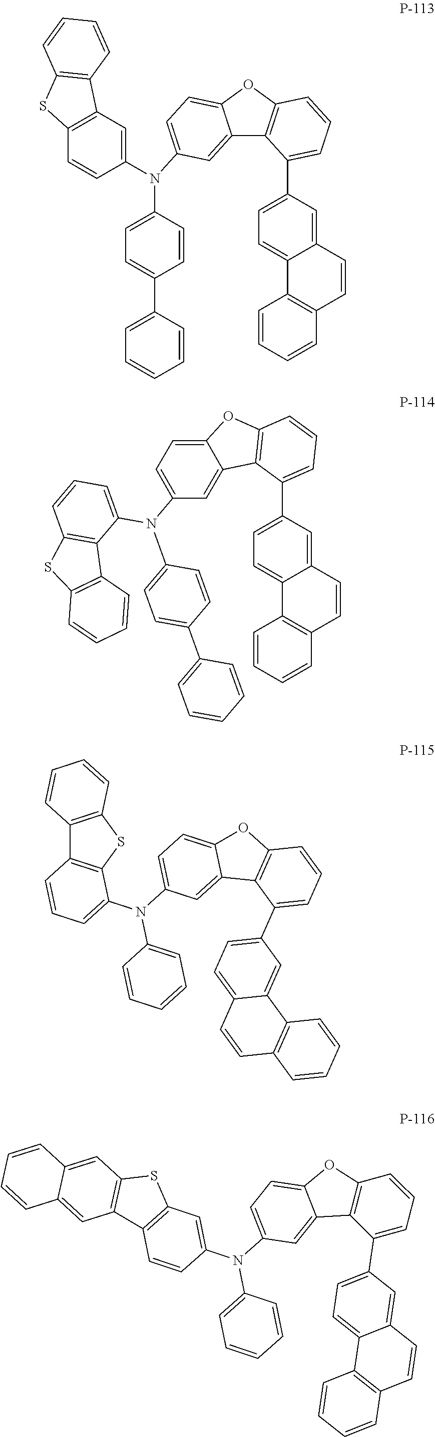 [SMILES: c1ccc(-c2ccc(N(c3ccc4sc5ccccc5c4c3)c3ccc4oc5cccc(-c6ccc7c(ccc8ccccc87)c6)c5c4c3)cc2)cc1 ; c1ccc(-c2ccc(N(c3ccc4oc5cccc(-c6ccc7c(ccc8ccccc87)c6)c5c4c3)c3cccc4sc5ccccc5c34)cc2)cc1 ; c1ccc(N(c2ccc3oc4cccc(-c5ccc6ccc7ccccc7c6c5)c4c3c2)c2cccc3c2sc2ccccc23)cc1 ; c1ccc(N(c2ccc3c(c2)sc2cc4ccccc4cc23)c2ccc3oc4cccc(-c5ccc6c(ccc7ccccc76)c5)c4c3c2)cc1]

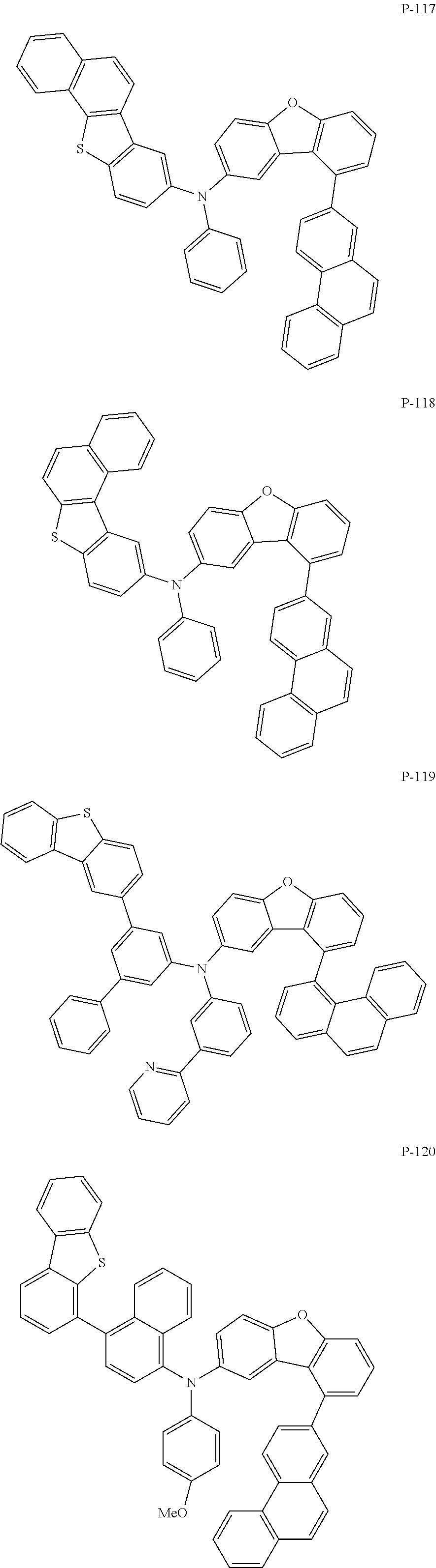 [SMILES: c1ccc(N(c2ccc3sc4c5ccccc5ccc4c3c2)c2ccc3oc4cccc(-c5ccc6c(ccc7ccccc76)c5)c4c3c2)cc1 ; c1ccc(N(c2ccc3oc4cccc(-c5ccc6c(ccc7ccccc76)c5)c4c3c2)c2ccc3sc4ccc5ccccc5c4c3c2)cc1 ; c1ccc(-c2cc(-c3ccc4sc5ccccc5c4c3)cc(N(c3cccc(-c4ccccn4)c3)c3ccc4oc5cccc(-c6cccc7ccc8ccccc8c67)c5c4c3)c2)cc1 ; COc1ccc(N(c2ccc3oc4cccc(-c5ccc6c(ccc7ccccc76)c5)c4c3c2)c2ccc(-c3cccc4c3sc3ccccc34)c3ccccc23)cc1]

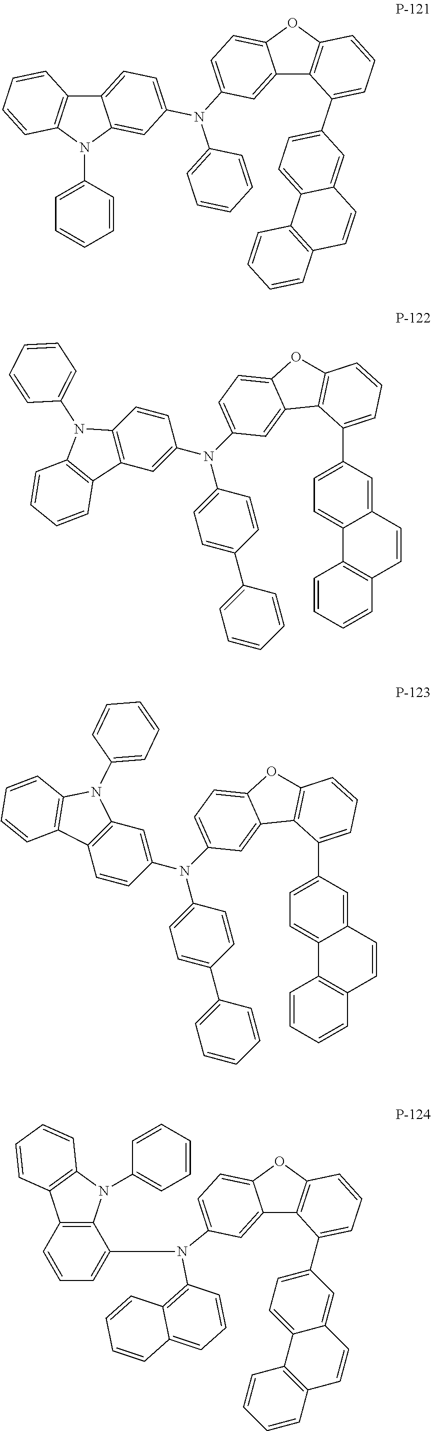 [SMILES: c1ccc(N(c2ccc3oc4cccc(-c5ccc6c(ccc7ccccc76)c5)c4c3c2)c2ccc3c4ccccc4n(-c4ccccc4)c3c2)cc1 ; c1ccc(-c2ccc(N(c3ccc4oc5cccc(-c6ccc7c(ccc8ccccc87)c6)c5c4c3)c3ccc4c(c3)c3ccccc3n4-c3ccccc3)cc2)cc1 ; c1ccc(-c2ccc(N(c3ccc4oc5cccc(-c6ccc7c(ccc8ccccc87)c6)c5c4c3)c3ccc4c5ccccc5n(-c5ccccc5)c4c3)cc2)cc1 ; c1ccc(-n2c3ccccc3c3cccc(N(c4ccc5oc6cccc(-c7ccc8c(ccc9ccccc98)c7)c6c5c4)c4cccc5ccccc45)c32)cc1]

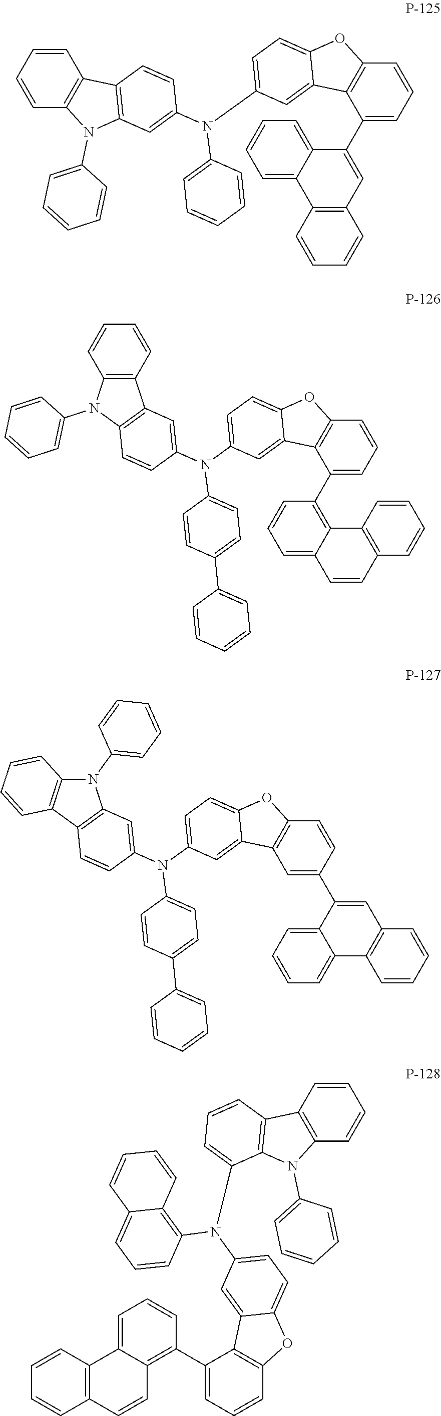 [SMILES: c1ccc(N(c2ccc3oc4cccc(-c5cc6ccccc6c6ccccc56)c4c3c2)c2ccc3c4ccccc4n(-c4ccccc4)c3c2)cc1 ; c1ccc(-c2ccc(N(c3ccc4oc5cccc(-c6cccc7ccc8ccccc8c67)c5c4c3)c3ccc4c(c3)c3ccccc3n4-c3ccccc3)cc2)cc1 ; c1ccc(-c2ccc(N(c3ccc4oc5ccc(-c6cc7ccccc7c7ccccc67)cc5c4c3)c3ccc4c5ccccc5n(-c5ccccc5)c4c3)cc2)cc1 ; c1ccc(-n2c3ccccc3c3cccc(N(c4ccc5oc6cccc(-c7cccc8c7ccc7ccccc78)c6c5c4)c4cccc5ccccc45)c32)cc1]

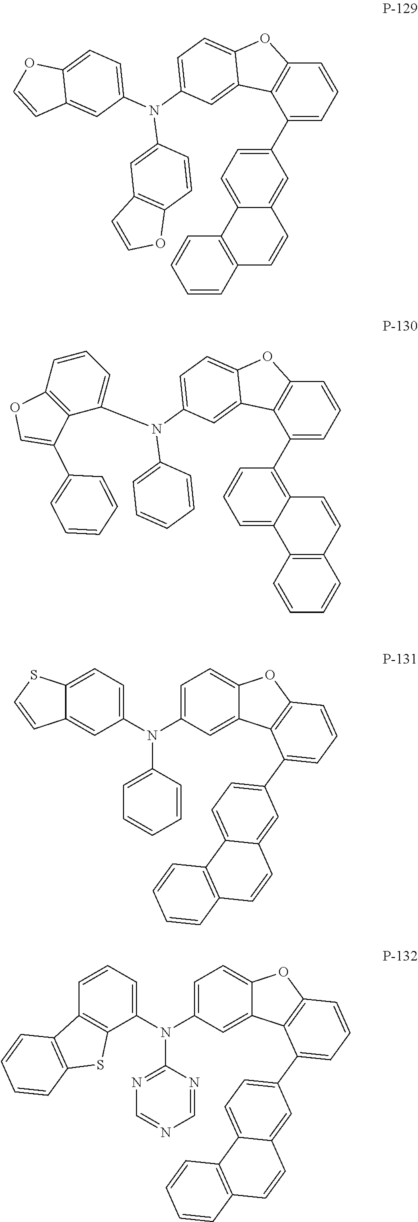 [SMILES: c1ccc2c(c1)ccc1cc(-c3cccc4oc5ccc(N(c6ccc7occc7c6)c6ccc7occc7c6)cc5c34)ccc12 ; c1ccc(-c2coc3cccc(N(c4ccccc4)c4ccc5oc6cccc(-c7cccc8c7ccc7ccccc78)c6c5c4)c23)cc1 ; c1ccc(N(c2ccc3sccc3c2)c2ccc3oc4cccc(-c5ccc6c(ccc7ccccc76)c5)c4c3c2)cc1 ; c1ccc2c(c1)ccc1cc(-c3cccc4oc5ccc(N(c6ncncn6)c6cccc7c6sc6ccccc67)cc5c34)ccc12]

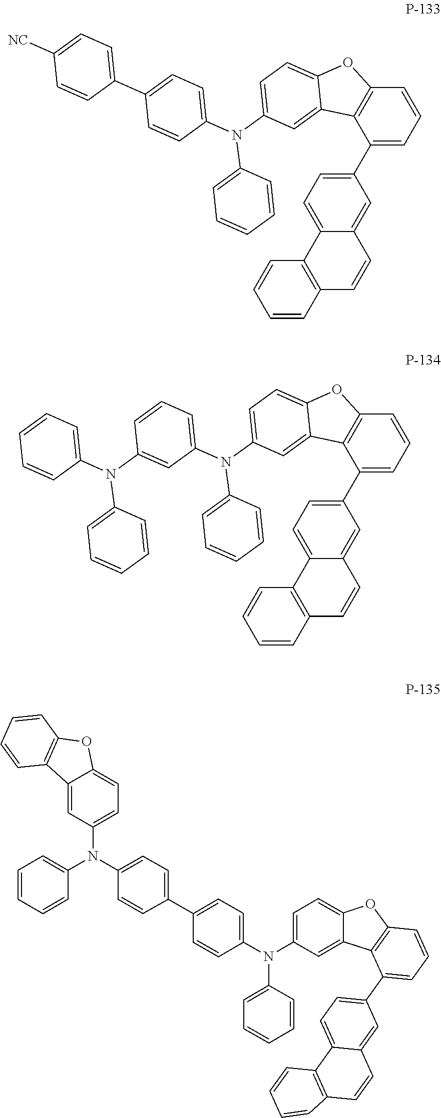 [SMILES: N#Cc1ccc(-c2ccc(N(c3ccccc3)c3ccc4oc5cccc(-c6ccc7c(ccc8ccccc87)c6)c5c4c3)cc2)cc1 ; c1ccc(N(c2ccccc2)c2cccc(N(c3ccccc3)c3ccc4oc5cccc(-c6ccc7c(ccc8ccccc87)c6)c5c4c3)c2)cc1 ; c1ccc(N(c2ccc(-c3ccc(N(c4ccccc4)c4ccc5oc6cccc(-c7ccc8c(ccc9ccccc98)c7)c6c5c4)cc3)cc2)c2ccc3oc4ccccc4c3c2)cc1]

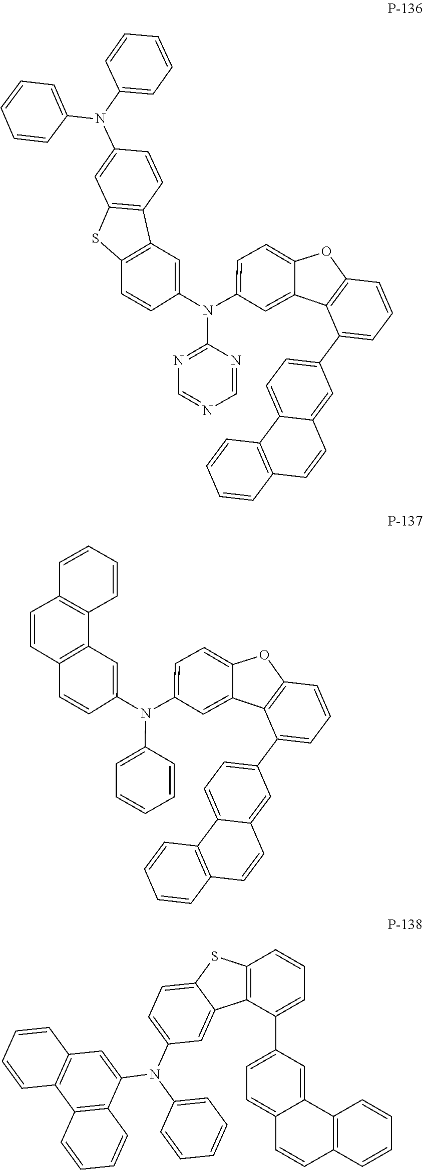 [SMILES: c1ccc(N(c2ccccc2)c2ccc3c(c2)sc2ccc(N(c4ccc5oc6cccc(-c7ccc8c(ccc9ccccc98)c7)c6c5c4)c4ncncn4)cc23)cc1 ; c1ccc(N(c2ccc3ccc4ccccc4c3c2)c2ccc3oc4cccc(-c5ccc6c(ccc7ccccc76)c5)c4c3c2)cc1 ; c1ccc(N(c2ccc3sc4cccc(-c5ccc6ccc7ccccc7c6c5)c4c3c2)c2cc3ccccc3c3ccccc23)cc1]

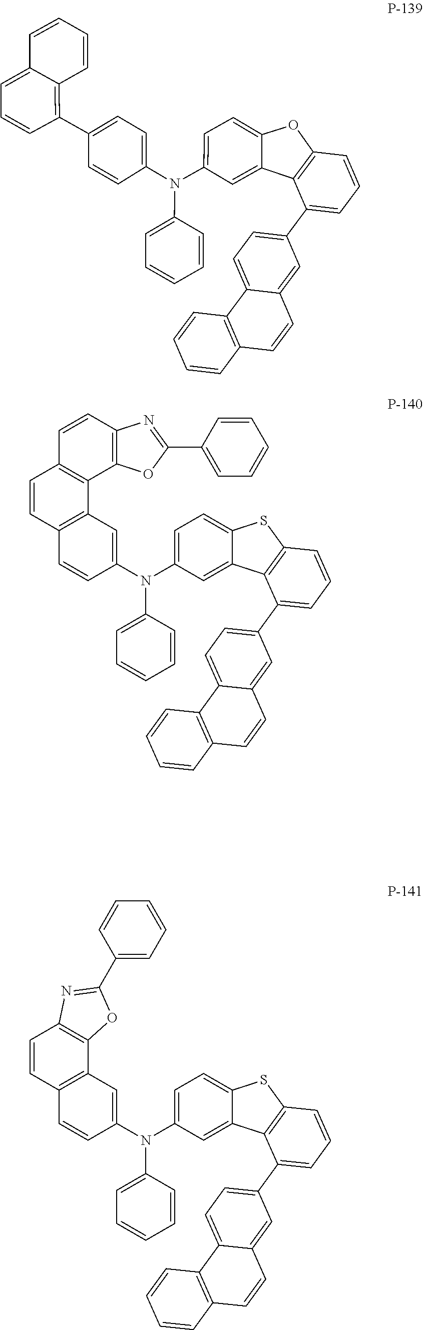

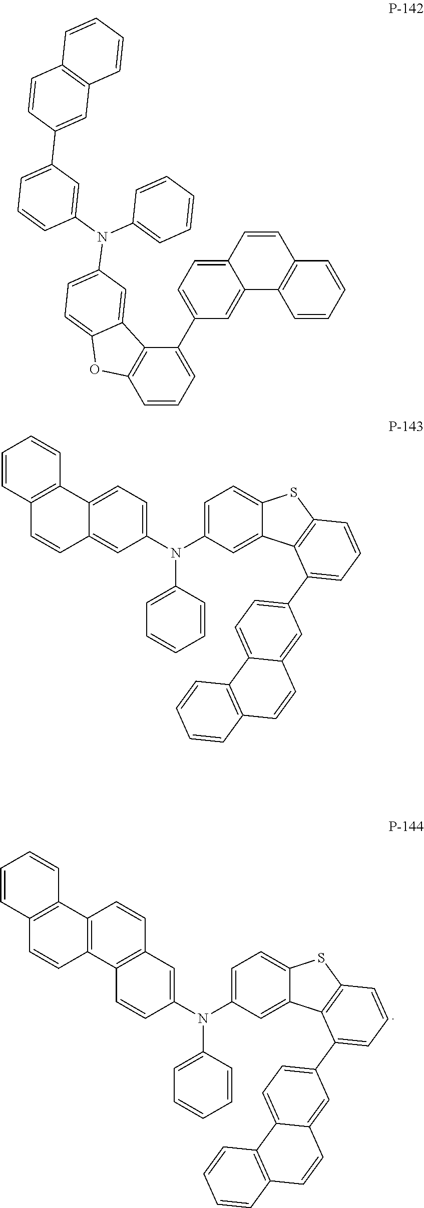 [SMILES: c1ccc(N(c2cccc(-c3ccc4ccccc4c3)c2)c2ccc3oc4cccc(-c5ccc6ccc7ccccc7c6c5)c4c3c2)cc1 ; c1ccc(N(c2ccc3c(ccc4ccccc43)c2)c2ccc3sc4cccc(-c5ccc6c(ccc7ccccc76)c5)c4c3c2)cc1 ; c1ccc(N(c2ccc3c(ccc4c5ccccc5ccc34)c2)c2ccc3sc4cccc(-c5ccc6c(ccc7ccccc76)c5)c4c3c2)cc1]

In another aspect of the present invention, the present invention provides an organic electric element comprising a first electrode, a second electrode, and an organic material layer between the first electrode and the second electrode, wherein the organic material layer comprises compound represented by the Formula 1.

The organic material layer includes a light-emitting layer and a hole transport zone between the first electrode and the light-emitting layer, and the light-emitting layer and/or the hole transport zone includes the compound represented by Formula 1. Preferably, the hole transport zone includes an emission auxiliary layer, and the compound of Formula 1 may be included in the light-emitting layer and/or the emission auxiliary layer.

The organic electric element may further include a layer for improving luminous efficiency. Here, the layer for improving luminous efficiency is formed on one side of a first electrode or a second electrode, the one side is not facing the organic material layer. Illustratively, the compound represented by Formula 1 may be included in the light efficiency improvement layer.

The organic material layer may comprise two or more stacks, wherein the stacks may comprise a hole transport layer, a light emitting layer, and an electron transport layer formed in sequence on the anode, and a charge generation layer may be formed between the two or more stacks.

In another aspect of the present invention, the present invention provides an electronic device comprising a display device and a control unit for controlling the display device, wherein the display device comprises the compound represented by Formula 1.

Hereinafter, synthesis example of the compound represented by Formula 1 and preparation method of an organic electroluminescent element will be described in detail by way of examples. However, the present invention is not limited to the following examples.

### Synthesis Example

The compound represented by Formula 1 according to the present invention can be synthesized by reacting Sub 1 and Sub 2 as shown in Reaction Scheme 1 below, but is not limited thereto.

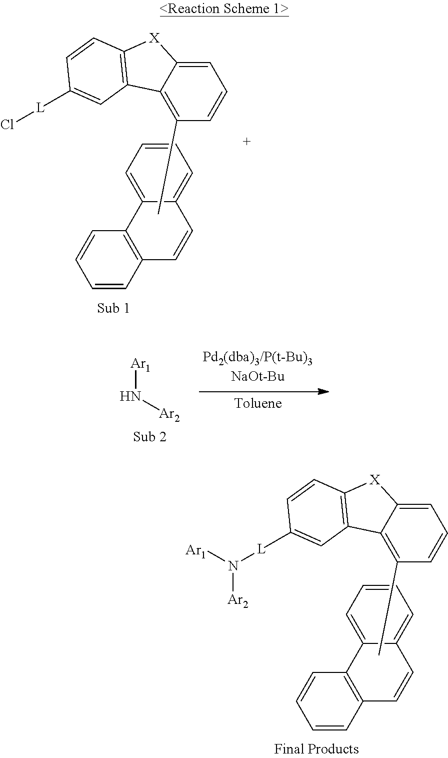

### Synthesis Example of Sub1

Sub 1 of the Reaction Scheme 1 can be synthesized according to the reaction route of the following Reaction Scheme 2, but is not limited thereto.

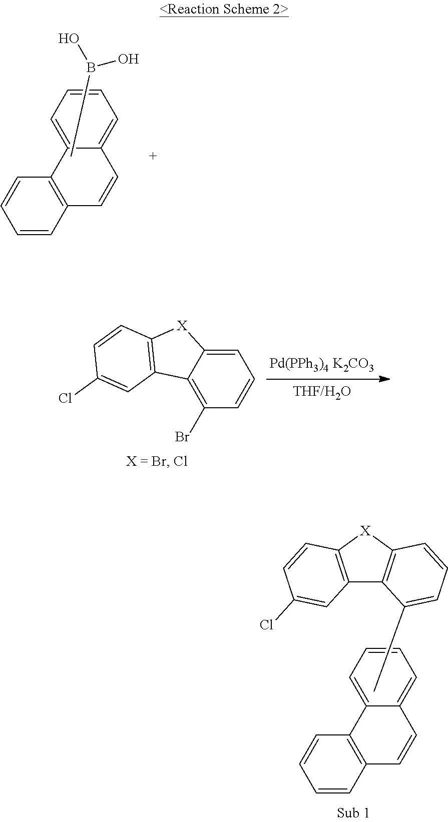

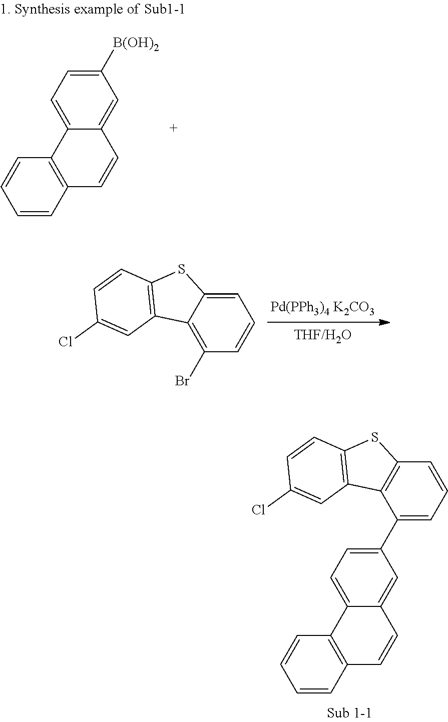 [SMILES: OB(O)c1ccc2c(ccc3ccccc32)c1 ; "+" ; Clc1ccc2sc3cccc(Br)c3c2c1 ; Clc1ccc2sc3cccc(-c4ccc5c(ccc6ccccc65)c4)c3c2c1]

After dissolving 1-bromo-8-chlorodibenzo[b,d]thiophene (CAS No. 1956366-55-4) (20 g, 67.2 mmol) in THF (200 ml), phenanthren-2-ylboronic acid (22.4 g 100.8 mmol), Pd(PPh₃)₄ (2.3 g, 2.0 mmol), K₂CO₃ (34.0 g, 27.9 mmol) and water (60 ml) were added to the solution and the mixture was stirred at 80° C. When the reaction was completed, the reaction product was extracted with CH₂Cl₂ and water, and then an organic layer was dried with MgSO₄ and concentrated. Then, the concentrate was separated through a silica gel column and recrystallized to obtain 21 g (yield: 79%) of the product.

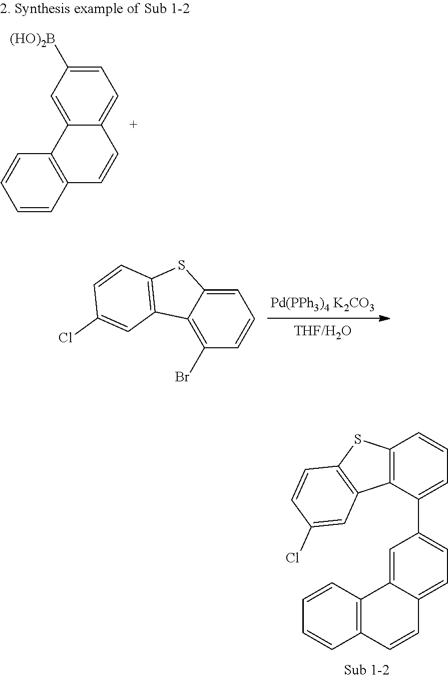

After dissolving 1-bromo-8-chlorodibenzo[b,d]thiophene (CAS No. 1956366-55-4) (25 g, 84.0 mmol) in THF (210 ml), phenanthren-3-ylboronic acid (28.0 g 126.0 mmol), Pd(PPh₃)₄ (2.9 g, 2.5 mmol), K₂CO₃ (34.8 g, 252.0 mmol) and water (70 ml) were added thereto, and then the synthesis was carried out in the same manner as in the synthesis method of Sub 1-1 to obtain 25 g (yield: 75.4%) of the product.

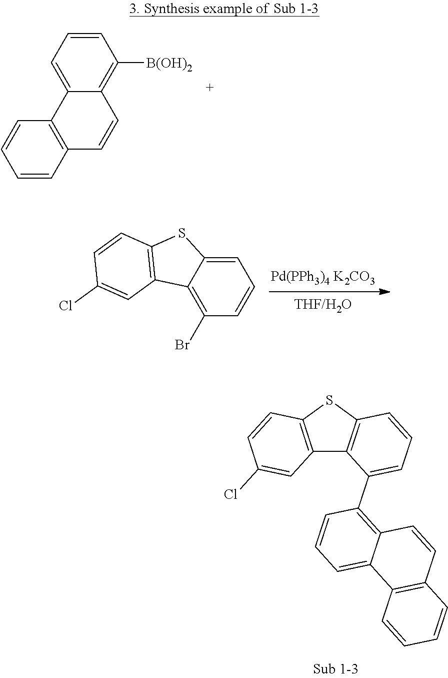 [SMILES: OB(O)c1cccc2c1ccc1ccccc12 ; Clc1ccc2sc3cccc(Br)c3c2c1 ; Clc1ccc2sc3cccc(-c4cccc5c4ccc4ccccc45)c3c2c1]

After dissolving 1-bromo-8-chlorodibenzo[b,d]thiophene (CAS No. 1956366-55-4) (23 g, 77.3 mmol) in THF (200 ml), phenanthren-1-ylboronic acid (25.7 g 115.9 mmol), Pd(PPh₃)₄ (2.7 g, 2.3 mmol), K₂CO₃ (32.0 g, 231.9 mmol) and water (70 ml) were added thereto, and then the synthesis was carried out in the same manner as in the synthesis method of Sub 1-1 to obtain 21.2 g (yield: 69.2%) of the product.

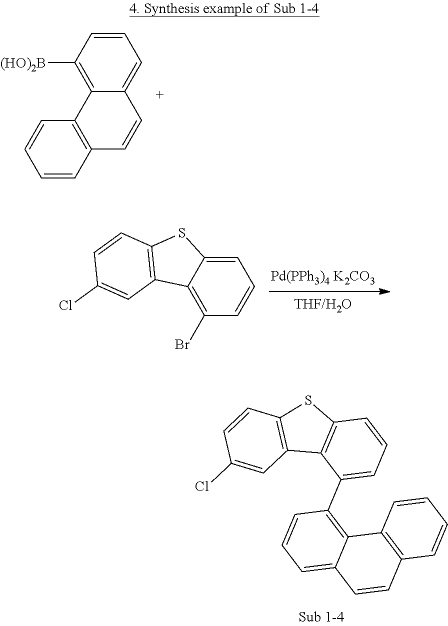 [SMILES: OB(O)c1cccc2ccc3ccccc3c12 ; Clc1ccc2sc3cccc(Br)c3c2c1 ; Clc1ccc2sc3cccc(-c4cccc5ccc6ccccc6c45)c3c2c1]

After dissolving 1-bromo-8-chlorodibenzo[b,d]thiophene (CAS No. 1956366-55-4) (30 g, 100.8 mmol) in THF (290 ml), phenanthren-4-ylboronic acid (33.6 g 151.2 mmol), Pd(PPh₃)₄ (3.5 g, 3.0 mmol), K₂CO₃ (41.8 g, 301.4 mmol) and water (100 ml) were added thereto, and then the synthesis was carried out in the same manner as in the synthesis method of Sub 1-1 to obtain 29.9 g (yield: 75.2%) of the product.

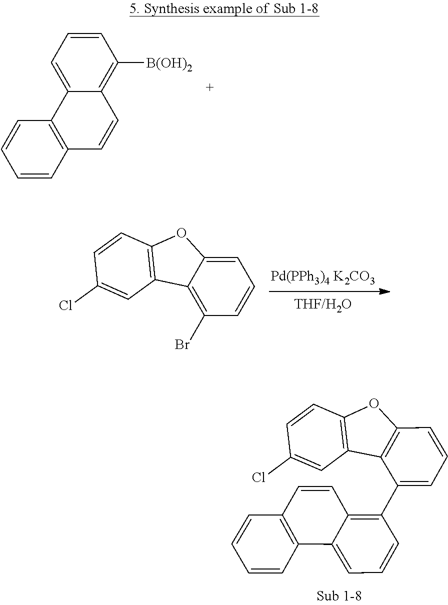 [SMILES: OB(O)c1cccc2c1ccc1ccccc12 ; Clc1ccc2oc3cccc(Br)c3c2c1 ; Clc1ccc2oc3cccc(-c4cccc5c4ccc4ccccc45)c3c2c1]

After dissolving 1-bromo-8-chlorodibenzo[b,d]furan (CAS No. 2173554-83-9) (20 g, 71.0 mmol) in THF (200 ml), phenanthren-1-ylboronic acid (23.7 g 106.6 mmol), Pd(PPh₃)₄ (2.5 g, 2.1 mmol), K₂CO₃ (29.5 g, 23.1 mmol) and water (60 ml) were added thereto, and then the synthesis was carried out in the same manner as in the synthesis method of Sub 1-1 to obtain 19.5 g (yield: 72.5%) of the product.

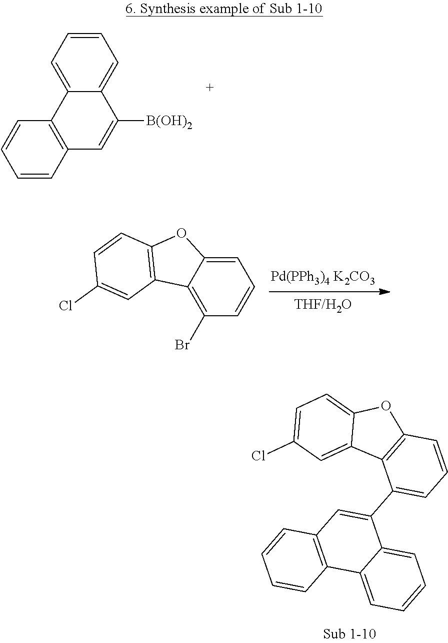 [SMILES: OB(O)c1cc2ccccc2c2ccccc12 ; Clc1ccc2oc3cccc(Br)c3c2c1 ; Clc1ccc2oc3cccc(-c4cc5ccccc5c5ccccc45)c3c2c1]

After dissolving 1-bromo-8-chlorodibenzo[b,d]furan (CAS No. 2173554-83-9) (25 g, 88.8 mmol) in THF (250 ml), phenanthren-9-ylboronic acid (29.6 g 133.2 mmol), Pd(PPh₃)₄ (3.1 g, 2.7 mmol), K₂CO₃ (36.8 g, 266.4 mmol) and water (80 ml) were added thereto, and then the synthesis was carried out in the same manner as in the synthesis method of Sub 1-1 to obtain 28 g (yield: 83.2%) of the product.

Compounds belonging to Sub 1 may be, but not limited to, the following compounds, and Table 1 shows FD-MS (Field Desorption-Mass Spectrometry) values of the following compounds.

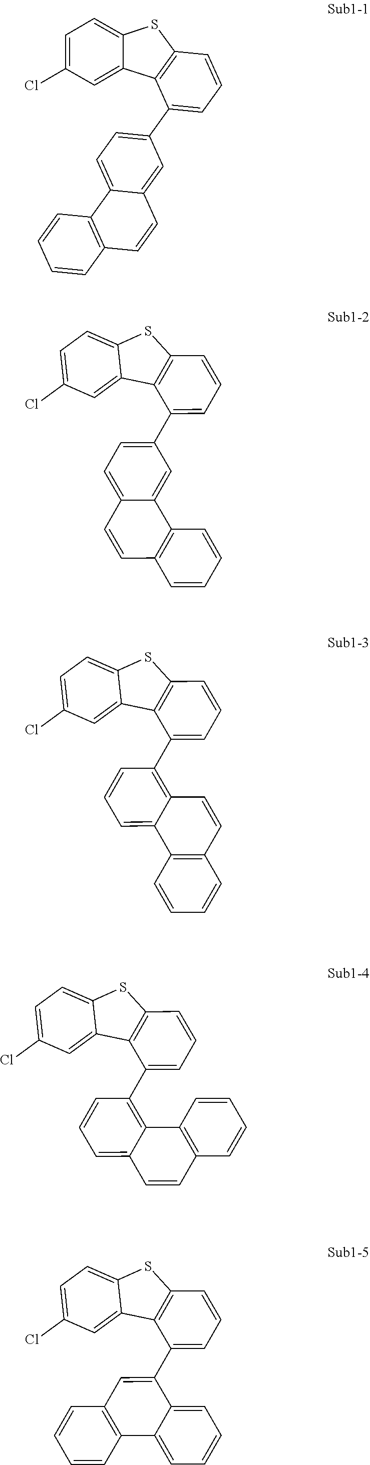 [SMILES: Clc1ccc2sc3cccc(-c4ccc5c(ccc6ccccc65)c4)c3c2c1 ; Clc1ccc2sc3cccc(-c4ccc5ccc6ccccc6c5c4)c3c2c1 ; Clc1ccc2sc3cccc(-c4cccc5c4ccc4ccccc45)c3c2c1 ; Clc1ccc2sc3cccc(-c4cccc5ccc6ccccc6c45)c3c2c1 ; Clc1ccc2sc3cccc(-c4cc5ccccc5c5ccccc45)c3c2c1]

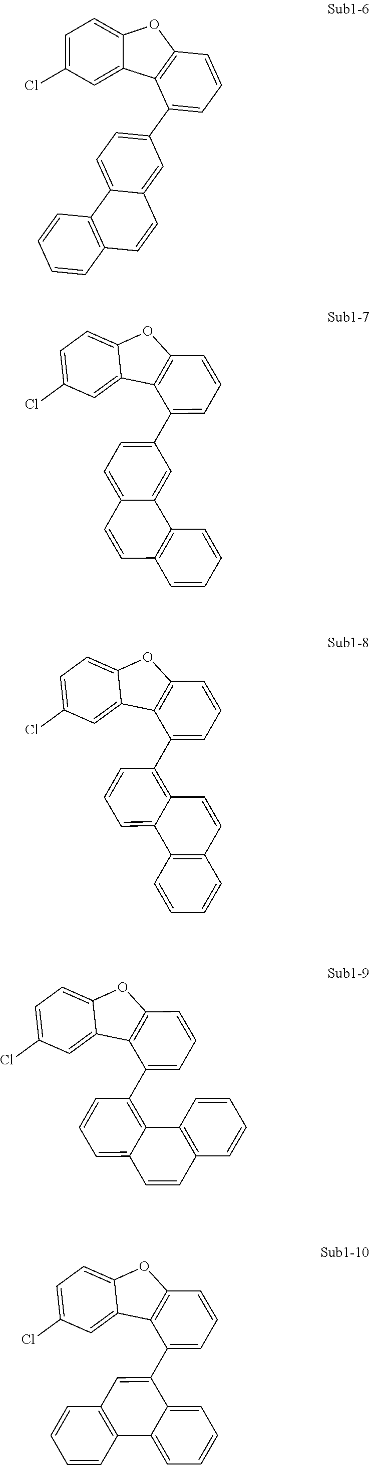 [SMILES: Clc1ccc2oc3cccc(-c4ccc5c(ccc6ccccc65)c4)c3c2c1 ; Clc1ccc2oc3cccc(-c4ccc5ccc6ccccc6c5c4)c3c2c1 ; Clc1ccc2oc3cccc(-c4cccc5c4ccc4ccccc45)c3c2c1 ; Clc1ccc2oc3cccc(-c4cccc5ccc6ccccc6c45)c3c2c1 ; Clc1ccc2oc3cccc(-c4cc5ccccc5c5ccccc45)c3c2c1]

TABLE 1

| Compound | FD-MS |
|---|---|
| Sub 1-1 | m/z = 394.06 (C₂₆H₁₅ClS = 394.92) |
| Sub 1-3 | m/z = 394.06 (C₂₆H₁₅ClS = 394.92) |
| Sub 1-5 | m/z = 394.06 (C₂₆H₁₅ClS = 394.92) |
| Sub 1-7 | m/z = 378.08 (C₂₆H₁₅ClO = 378.86) |
| Sub 1-9 | m/z = 378.08 (C₂₆H₁₅ClO = 378.86) |
| Sub 1-2 | m/z = 394.06 (C₂₆H₁₅ClS = 394.92) |
| Sub 1-4 | m/z = 394.06 (C₂₆H₁₅ClS = 394.92) |
| Sub 1-6 | m/z = 378.08 (C₂₆H₁₅ClO = 378.86) |
| Sub 1-8 | m/z = 378.08 (C₂₆H₁₅ClO = 378.86) |
| Sub 1-10 | m/z = 378.08 (C₂₆H₁₅ClO = 378.86) |

### Exemplary Compounds of Sub 2

Compounds belonging to Sub 2 may be, but not limited to, the following compounds, and Table 2 shows FD-MS (Field Desorption-Mass Spectrometry) values of the following compounds.

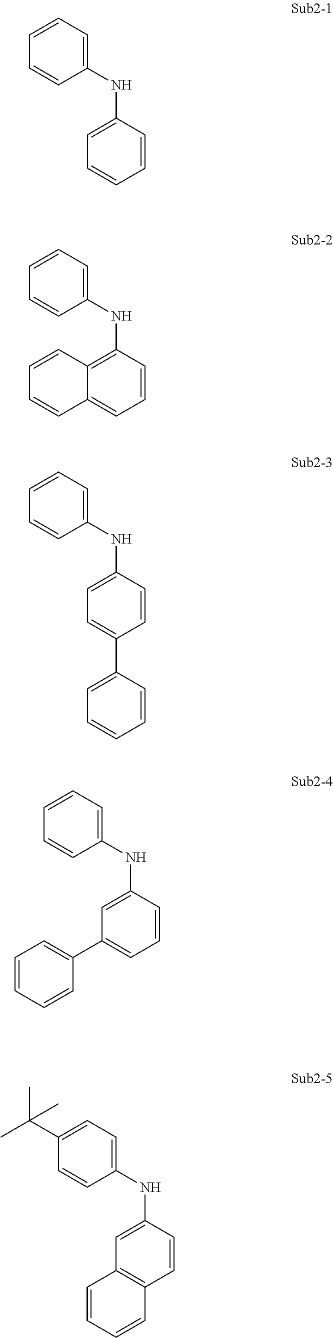 [SMILES: c1ccc(Nc2ccccc2)cc1 ; c1ccc(Nc2cccc3ccccc23)cc1 ; c1ccc(Nc2ccc(-c3ccccc3)cc2)cc1 ; c1ccc(Nc2cccc(-c3ccccc3)c2)cc1 ; CC(C)(C)c1ccc(Nc2ccc3ccccc3c2)cc1]

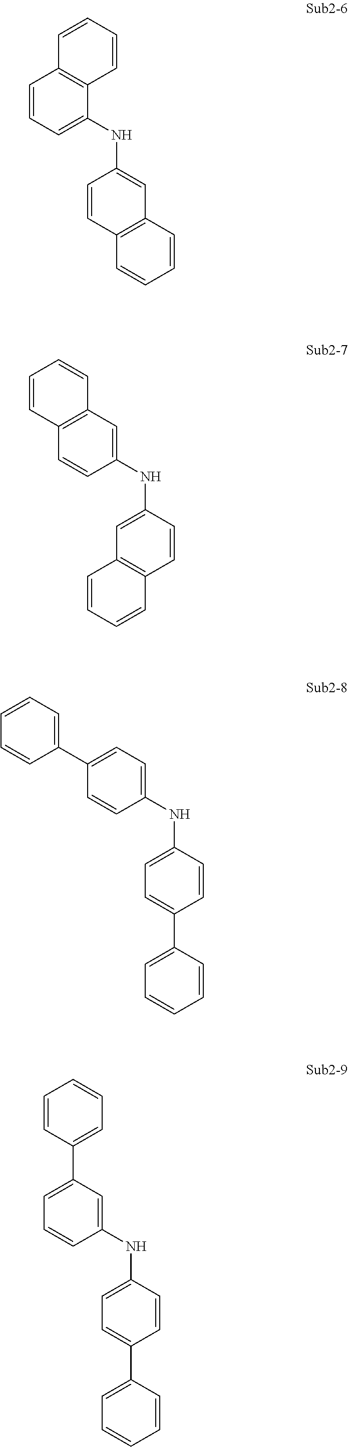 [SMILES: c1ccc2cc(Nc3cccc4ccccc34)ccc2c1 ; c1ccc2cc(Nc3ccc4ccccc4c3)ccc2c1 ; c1ccc(-c2ccc(Nc3ccc(-c4ccccc4)cc3)cc2)cc1 ; c1ccc(-c2ccc(Nc3cccc(-c4ccccc4)c3)cc2)cc1]

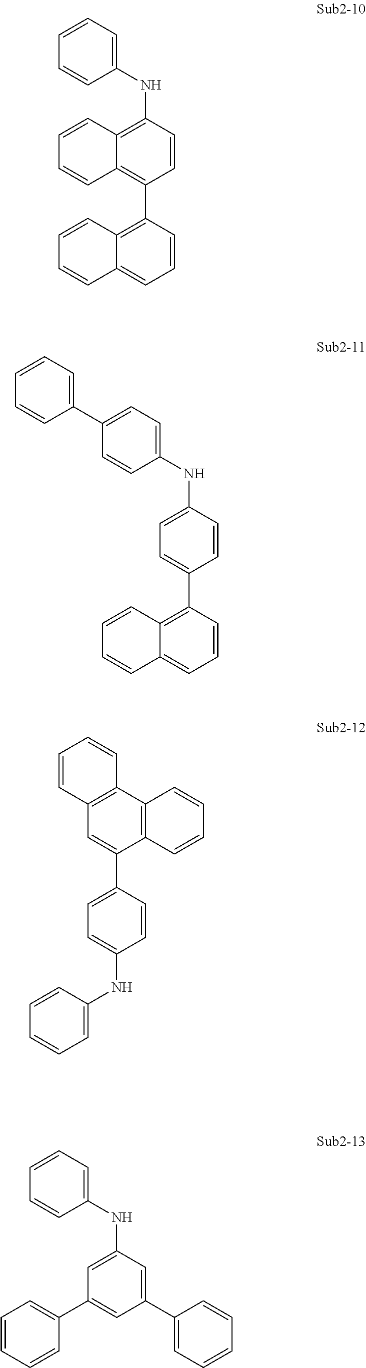 [SMILES: c1ccc(Nc2ccc(-c3cccc4ccccc34)c3ccccc23)cc1 ; c1ccc(-c2ccc(Nc3ccc(-c4cccc5ccccc45)cc3)cc2)cc1 ; c1ccc(Nc2ccc(-c3cc4ccccc4c4ccccc34)cc2)cc1 ; c1ccc(Nc2cc(-c3ccccc3)cc(-c3ccccc3)c2)cc1]

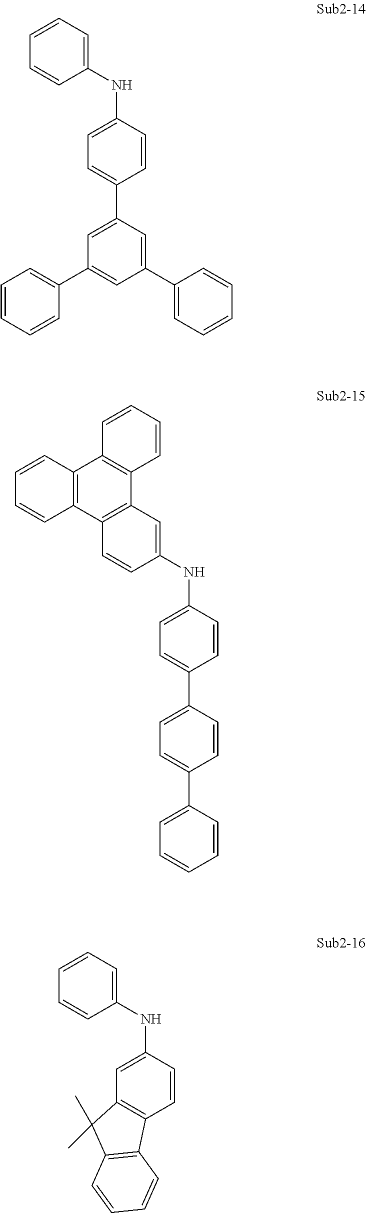 [SMILES: c1ccc(Nc2ccc(-c3cc(-c4ccccc4)cc(-c4ccccc4)c3)cc2)cc1 ; c1ccc(-c2ccc(-c3ccc(Nc4ccc5c6ccccc6c6ccccc6c5c4)cc3)cc2)cc1 ; CC1(C)c2ccccc2-c2ccc(Nc3ccccc3)cc21]

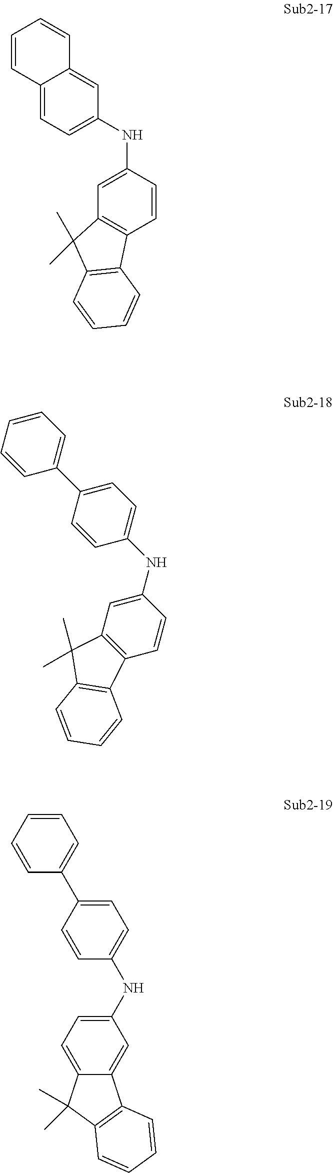 [SMILES: CC1(C)c2ccccc2-c2ccc(Nc3ccc4ccccc4c3)cc21 ; CC1(C)c2ccccc2-c2ccc(Nc3ccc(-c4ccccc4)cc3)cc21 ; CC1(C)c2ccccc2-c2cc(Nc3ccc(-c4ccccc4)cc3)ccc21]

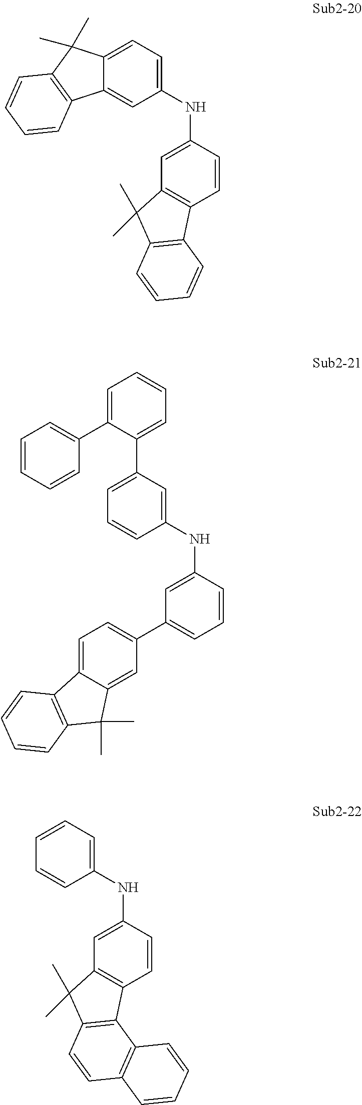 [SMILES: CC1(C)c2ccccc2-c2cc(Nc3ccc4c(c3)C(C)(C)c3ccccc3-4)ccc21 ; CC1(C)c2ccccc2-c2ccc(-c3cccc(Nc4cccc(-c5ccccc5-c5ccccc5)c4)c3)cc21 ; CC1(C)c2cc(Nc3ccccc3)ccc2-c2c1ccc1ccccc21]

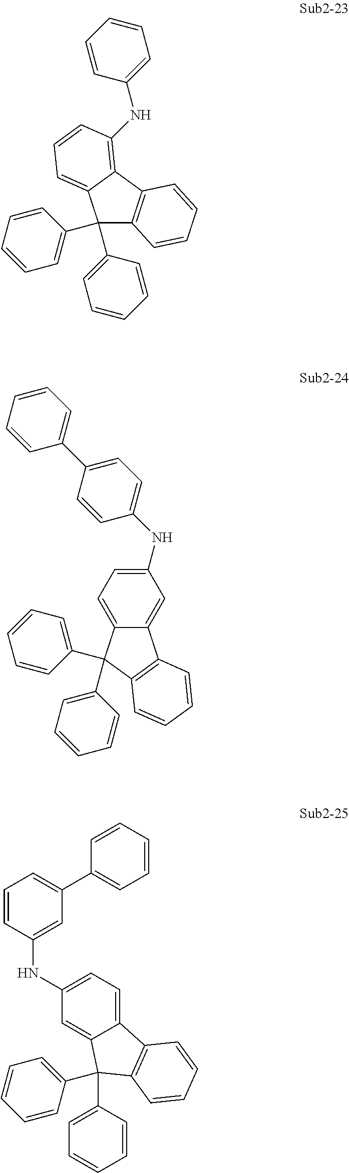 [SMILES: c1ccc(Nc2cccc3c2-c2ccccc2C3(c2ccccc2)c2ccccc2)cc1 ; c1ccc(-c2ccc(Nc3ccc4c(c3)-c3ccccc3C4(c3ccccc3)c3ccccc3)cc2)cc1 ; c1ccc(-c2cccc(Nc3ccc4c(c3)C(c3ccccc3)(c3ccccc3)c3ccccc3-4)c2)cc1]

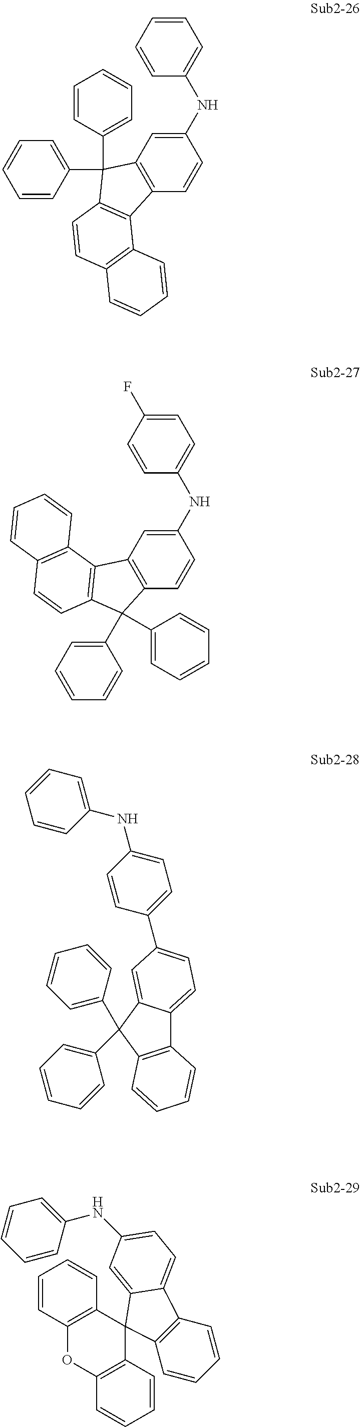 [SMILES: c1ccc(Nc2ccc3c(c2)C(c2ccccc2)(c2ccccc2)c2ccc4ccccc4c2-3)cc1 ; Fc1ccc(Nc2ccc3c(c2)-c2c(ccc4ccccc24)C3(c2ccccc2)c2ccccc2)cc1 ; c1ccc(Nc2ccc(-c3ccc4c(c3)C(c3ccccc3)(c3ccccc3)c3ccccc3-4)cc2)cc1 ; c1ccc(Nc2ccc3c(c2)C2(c4ccccc4Oc4ccccc42)c2ccccc2-3)cc1]

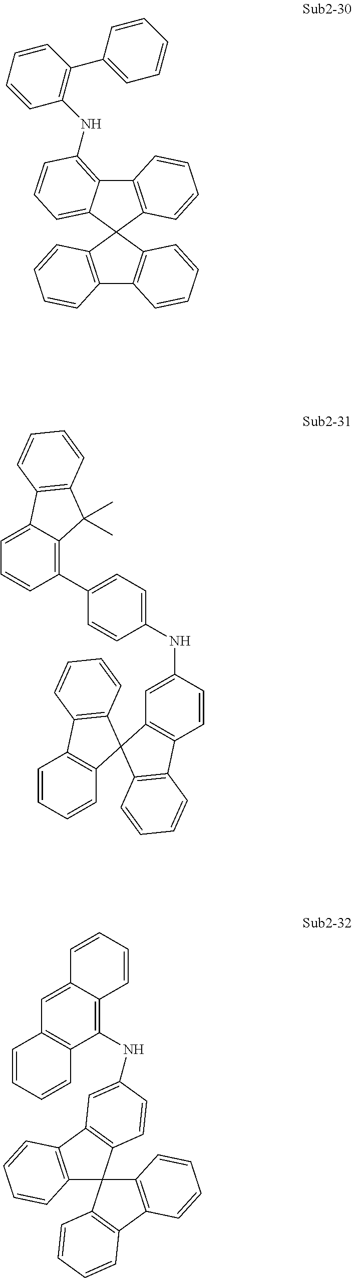 [SMILES: c1ccc(-c2ccccc2Nc2cccc3c2-c2ccccc2C32c3ccccc3-c3ccccc32)cc1 ; CC1(C)c2ccccc2-c2cccc(-c3ccc(Nc4ccc5c(c4)C4(c6ccccc6-c6ccccc64)c4ccccc4-5)cc3)c21 ; c1ccc2c(c1)-c1ccccc1C21c2ccccc2-c2cc(Nc3c4ccccc4cc4ccccc34)ccc21]

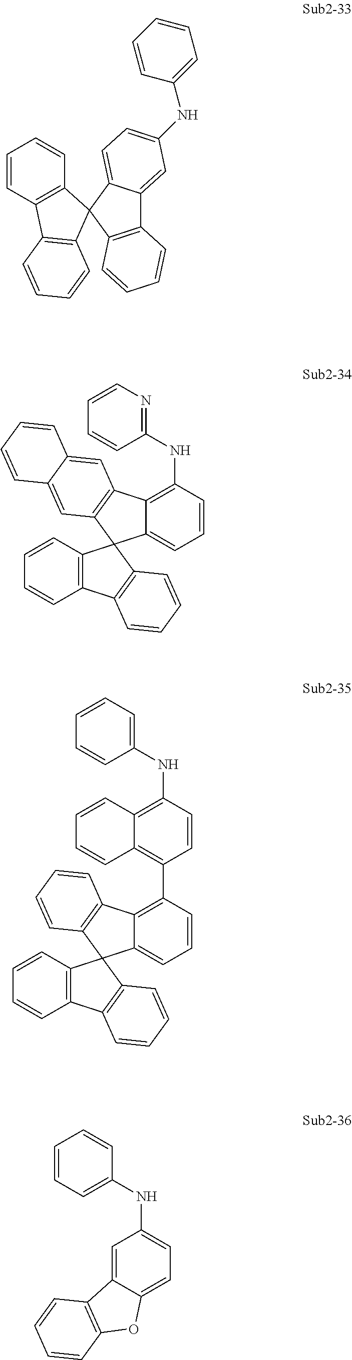 [SMILES: c1ccc(Nc2ccc3c(c2)-c2ccccc2C32c3ccccc3-c3ccccc32)cc1 ; c1ccc(Nc2cccc3c2-c2cc4ccccc4cc2C32c3ccccc3-c3ccccc32)nc1 ; c1ccc(Nc2ccc(-c3cccc4c3-c3ccccc3C43c4ccccc4-c4ccccc43)c3ccccc23)cc1 ; c1ccc(Nc2ccc3oc4ccccc4c3c2)cc1]

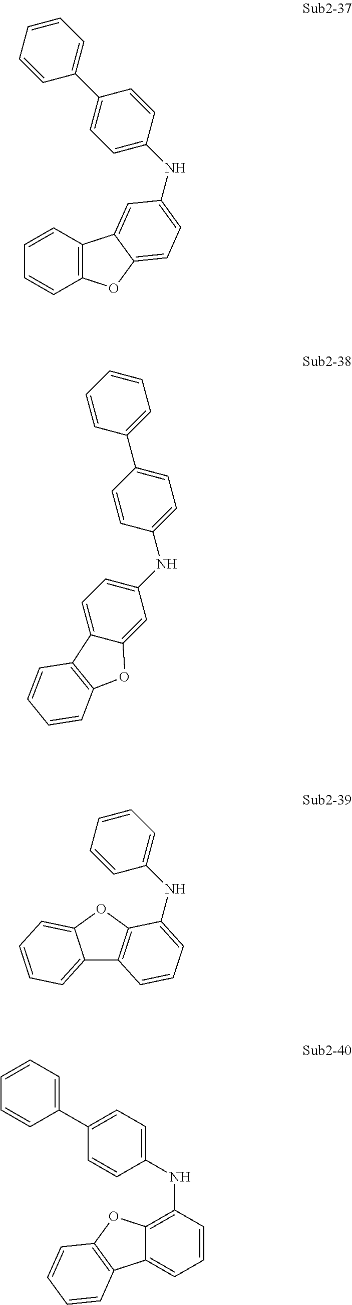 [SMILES: c1ccc(-c2ccc(Nc3ccc4oc5ccccc5c4c3)cc2)cc1 ; c1ccc(-c2ccc(Nc3ccc4c(c3)oc3ccccc34)cc2)cc1 ; c1ccc(Nc2cccc3c2oc2ccccc23)cc1 ; c1ccc(-c2ccc(Nc3cccc4c3oc3ccccc34)cc2)cc1]

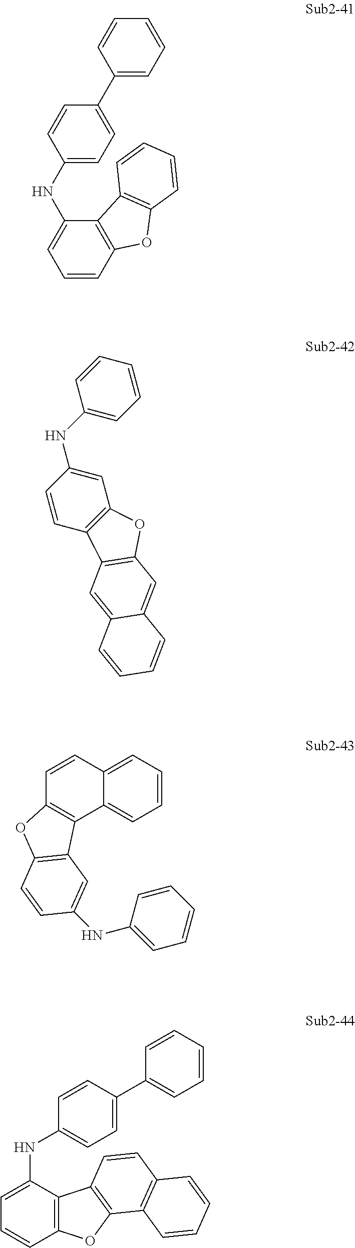 [SMILES: c1ccc(-c2ccc(Nc3cccc4oc5ccccc5c34)cc2)cc1 ; c1ccc(Nc2ccc3c(c2)oc2cc4ccccc4cc23)cc1 ; c1ccc(Nc2ccc3oc4ccc5ccccc5c4c3c2)cc1 ; c1ccc(-c2ccc(Nc3cccc4oc5c6ccccc6ccc5c34)cc2)cc1]

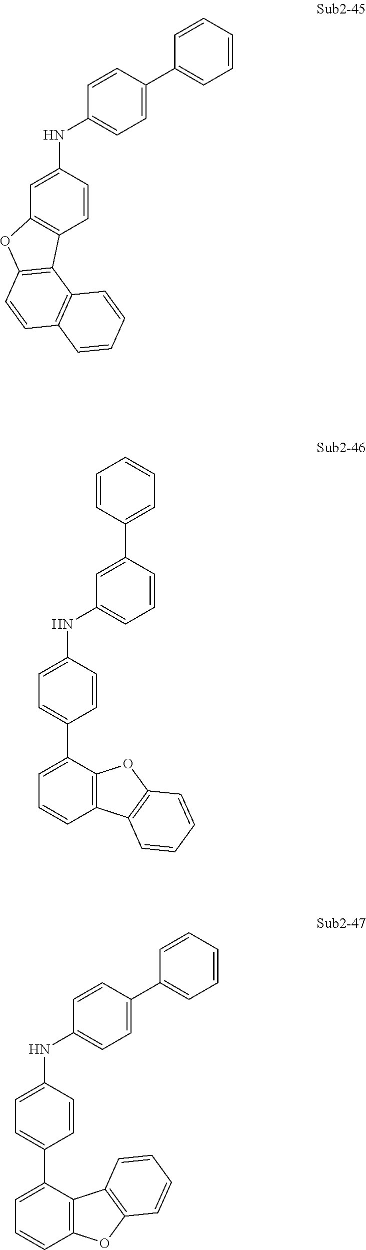 [SMILES: c1ccc(-c2ccc(Nc3ccc4c(c3)oc3ccc5ccccc5c34)cc2)cc1 ; c1ccc(-c2cccc(Nc3ccc(-c4cccc5c4oc4ccccc45)cc3)c2)cc1 ; c1ccc(-c2ccc(Nc3ccc(-c4cccc5oc6ccccc6c45)cc3)cc2)cc1]

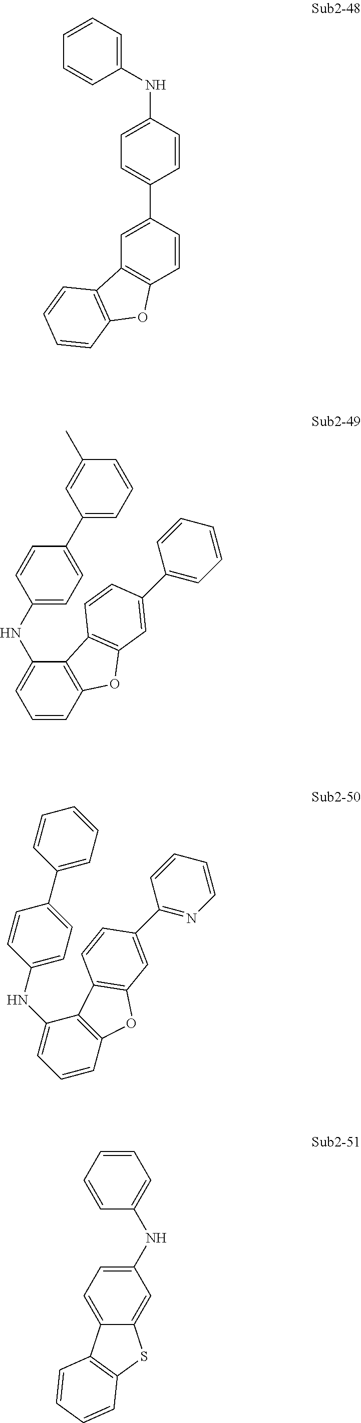 [SMILES: c1ccc(Nc2ccc(-c3ccc4oc5ccccc5c4c3)cc2)cc1 ; Cc1cccc(-c2ccc(Nc3cccc4oc5cc(-c6ccccc6)ccc5c34)cc2)c1 ; c1ccc(-c2ccc(Nc3cccc4oc5cc(-c6ccccn6)ccc5c34)cc2)cc1 ; c1ccc(Nc2ccc3c(c2)sc2ccccc23)cc1]

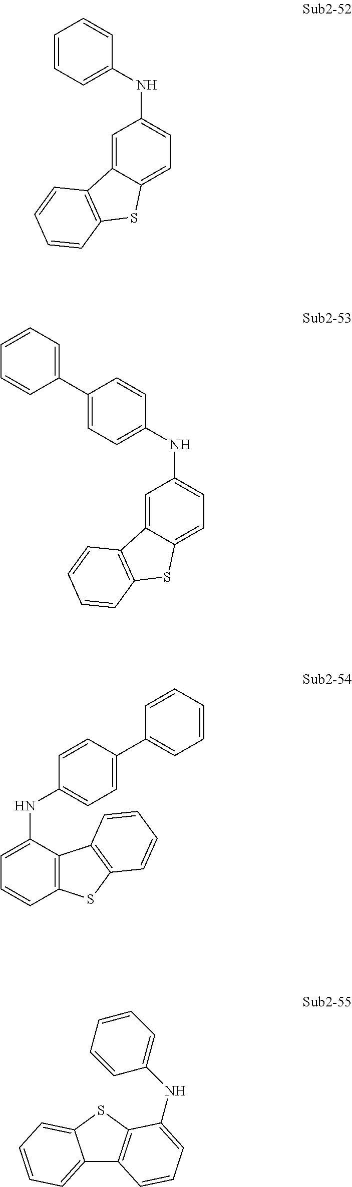 [SMILES: c1ccc(Nc2ccc3sc4ccccc4c3c2)cc1 ; c1ccc(-c2ccc(Nc3ccc4sc5ccccc5c4c3)cc2)cc1 ; c1ccc(-c2ccc(Nc3cccc4sc5ccccc5c34)cc2)cc1 ; c1ccc(Nc2cccc3c2sc2ccccc23)cc1]

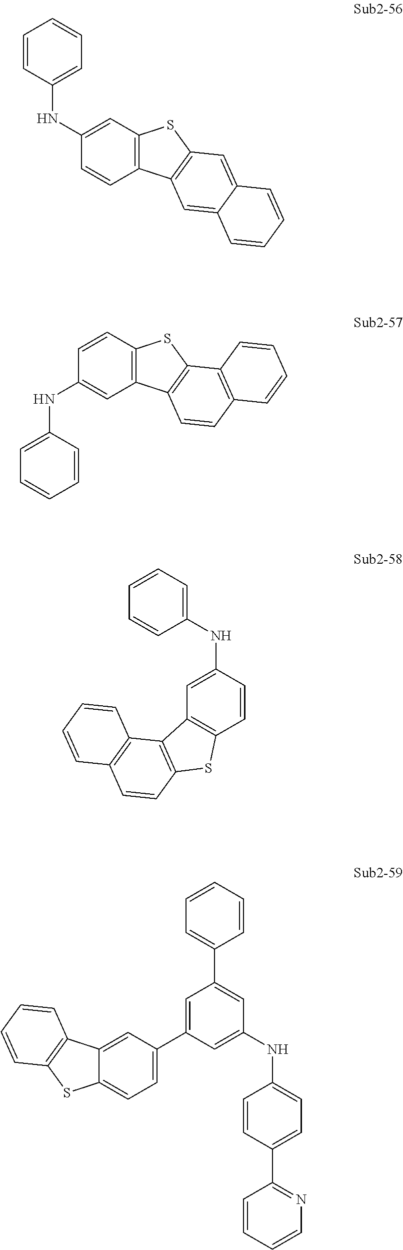 [SMILES: c1ccc(Nc2ccc3c(c2)sc2cc4ccccc4cc23)cc1 ; c1ccc(Nc2ccc3sc4c5ccccc5ccc4c3c2)cc1 ; c1ccc(Nc2ccc3sc4ccc5ccccc5c4c3c2)cc1 ; c1ccc(-c2cc(Nc3ccc(-c4ccccn4)cc3)cc(-c3ccc4sc5ccccc5c4c3)c2)cc1]

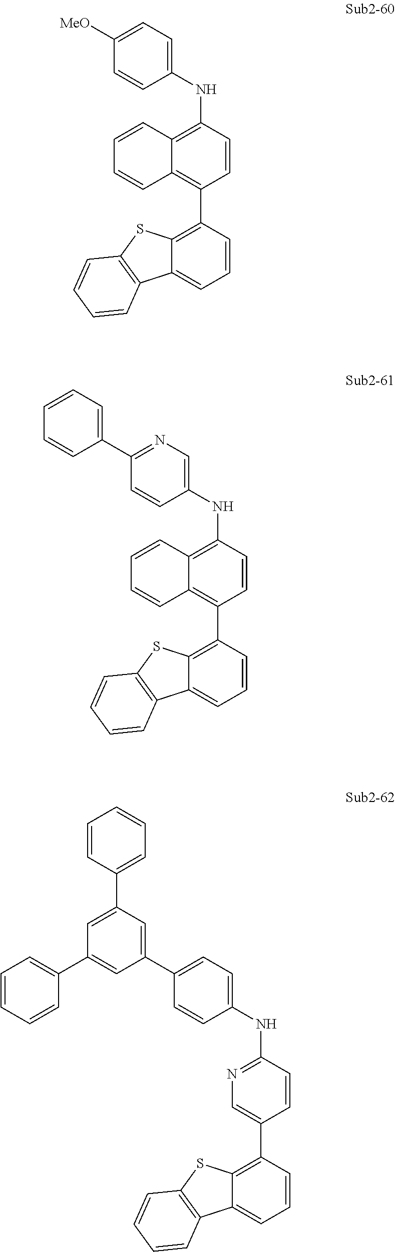 [SMILES: COc1ccc(Nc2ccc(-c3cccc4c3sc3ccccc34)c3ccccc23)cc1 ; c1ccc(-c2ccc(Nc3ccc(-c4cccc5c4sc4ccccc45)c4ccccc34)cn2)cc1 ; c1ccc(-c2cc(-c3ccccc3)cc(-c3ccc(Nc4ccc(-c5cccc6c5sc5ccccc56)cn4)cc3)c2)cc1]

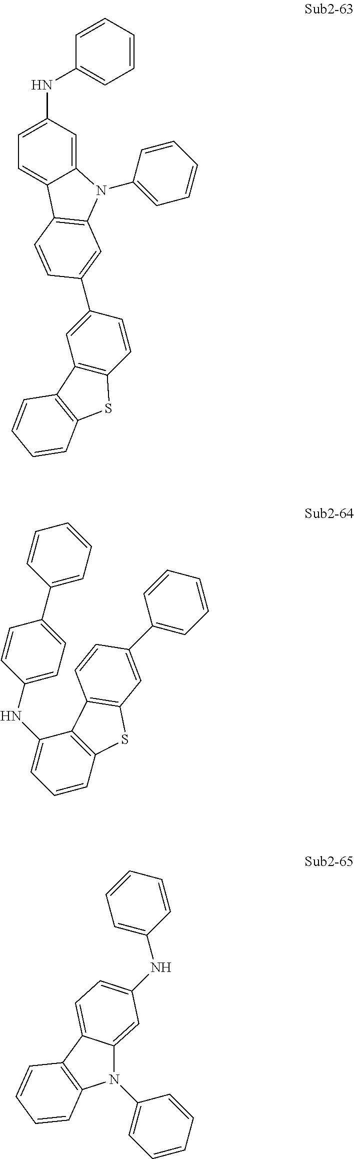 [SMILES: c1ccc(Nc2ccc3c4ccc(-c5ccc6sc7ccccc7c6c5)cc4n(-c4ccccc4)c3c2)cc1 ; c1ccc(-c2ccc(Nc3cccc4sc5cc(-c6ccccc6)ccc5c34)cc2)cc1 ; c1ccc(Nc2ccc3c4ccccc4n(-c4ccccc4)c3c2)cc1]

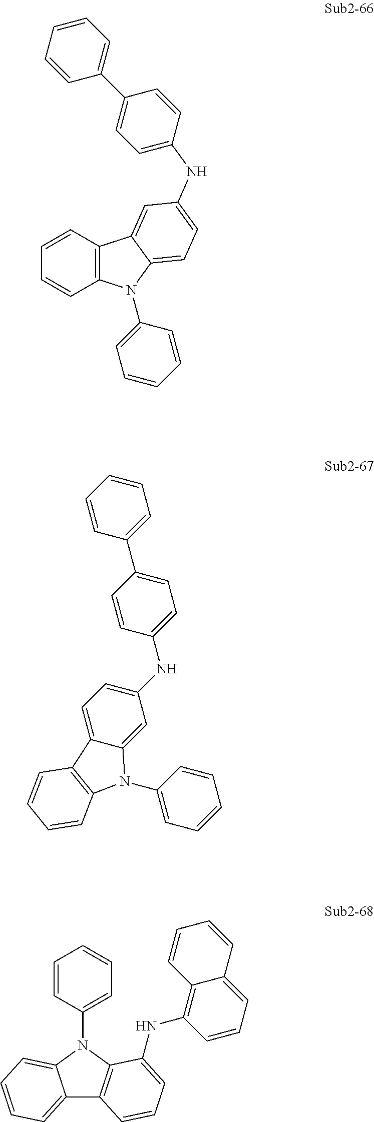 [SMILES: c1ccc(-c2ccc(Nc3ccc4c(c3)c3ccccc3n4-c3ccccc3)cc2)cc1 ; c1ccc(-c2ccc(Nc3ccc4c5ccccc5n(-c5ccccc5)c4c3)cc2)cc1 ; c1ccc(-n2c3ccccc3c3cccc(Nc4cccc5ccccc45)c32)cc1]

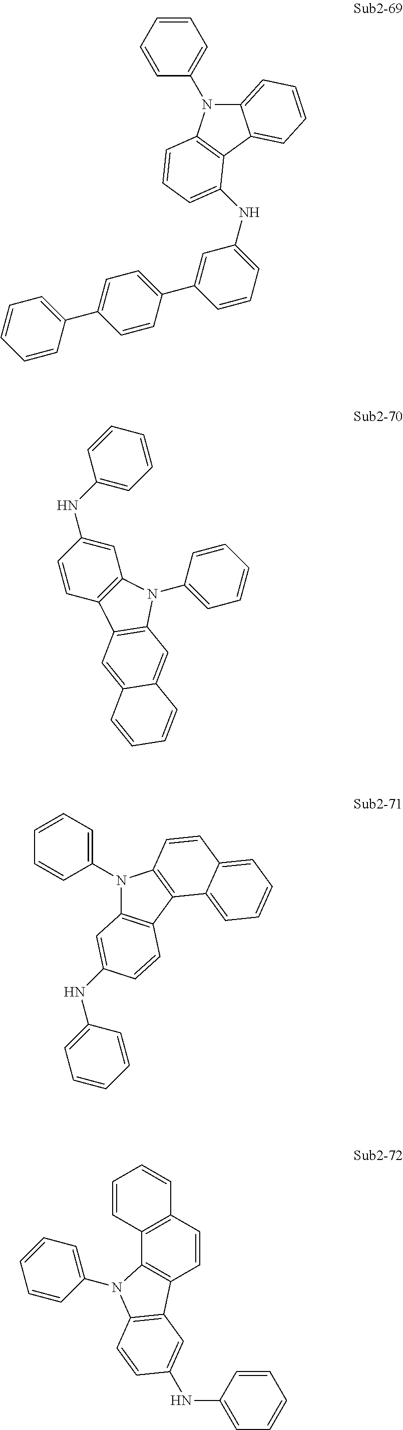 [SMILES: c1ccc(-c2ccc(-c3cccc(Nc4cccc5c4c4ccccc4n5-c4ccccc4)c3)cc2)cc1 ; c1ccc(Nc2ccc3c4cc5ccccc5cc4n(-c4ccccc4)c3c2)cc1 ; c1ccc(Nc2ccc3c4c5ccccc5ccc4n(-c4ccccc4)c3c2)cc1 ; c1ccc(Nc2ccc3c(c2)c2ccc4ccccc4c2n3-c2ccccc2)cc1]

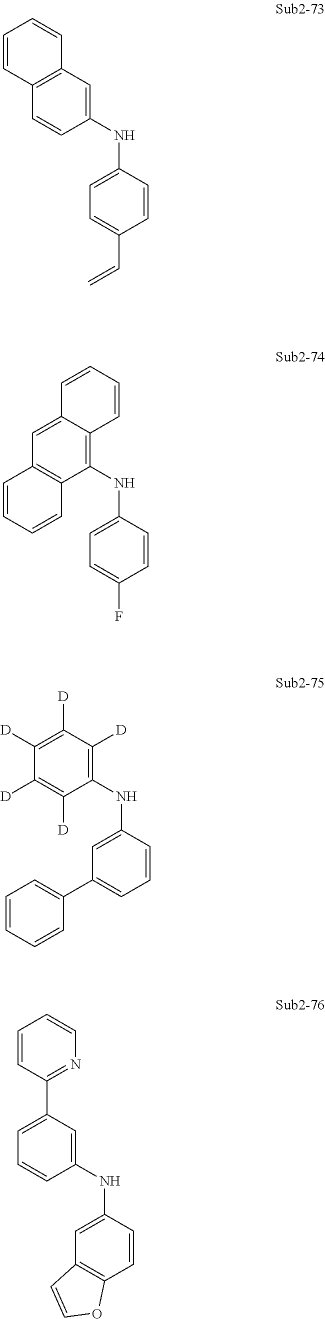 [SMILES: C=Cc1ccc(Nc2ccc3ccccc3c2)cc1 ; Fc1ccc(Nc2c3ccccc3cc3ccccc23)cc1 ; [2H]c1c([2H])c([2H])c(Nc2cccc(-c3ccccc3)c2)c([2H])c1[2H] ; c1ccc(-c2cccc(Nc3ccc4occc4c3)c2)nc1]

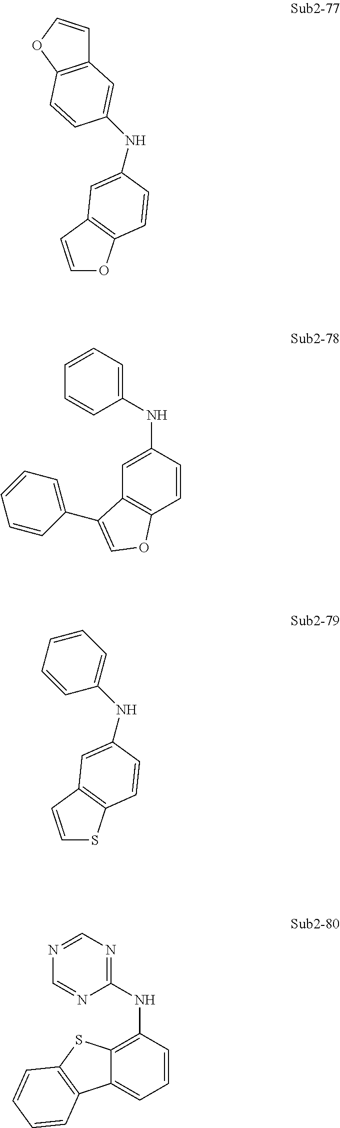 [SMILES: c1cc2cc(Nc3ccc4occc4c3)ccc2o1 ; c1ccc(Nc2ccc3occ(-c4ccccc4)c3c2)cc1 ; c1ccc(Nc2ccc3sccc3c2)cc1 ; c1ccc2c(c1)sc1c(Nc3ncncn3)cccc12]

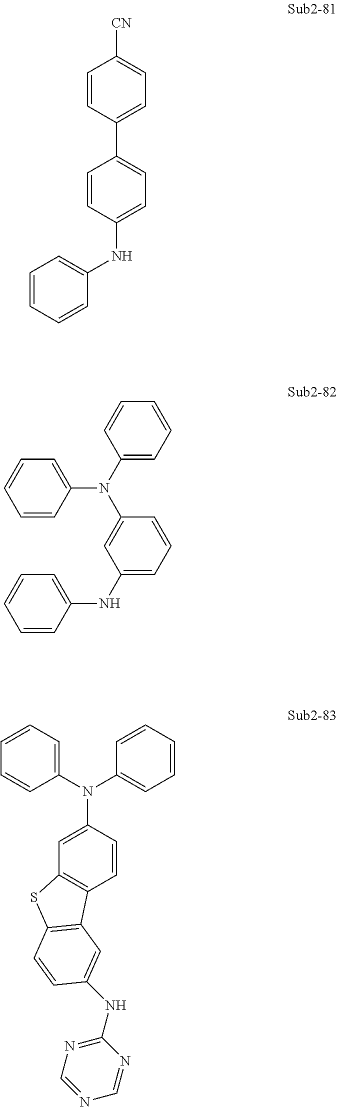 [SMILES: N#Cc1ccc(-c2ccc(Nc3ccccc3)cc2)cc1 ; c1ccc(Nc2cccc(N(c3ccccc3)c3ccccc3)c2)cc1 ; c1ccc(N(c2ccccc2)c2ccc3c(c2)sc2ccc(Nc4ncncn4)cc23)cc1]

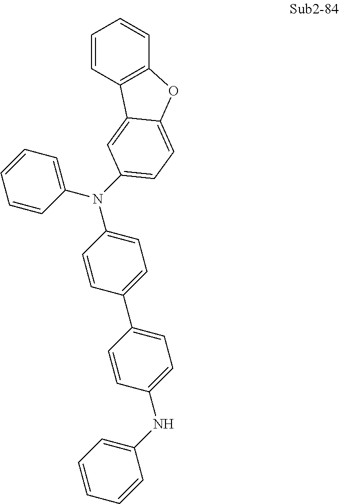 [SMILES: c1ccc(Nc2ccc(-c3ccc(N(c4ccccc4)c4ccc5oc6ccccc6c5c4)cc3)cc2)cc1]

TABLE 2

| Compound | FD-MS |
|---|---|
| Sub 2-1 | m/z = 169.09 (C₁₂H₁₁N = 169.23) |
| Sub 2-3 | m/z = 245.12 (C₁₈H₁₅N = 245.33) |
| Sub 2-5 | m/z = 275.17 (C₂₀H₂₁N = 275.4) |
| Sub 2-7 | m/z = 269.12 (C₂₀H₁₅N = 269.35) |
| Sub 2-9 | m/z = 321.15 (C₂₄H₁₉N = 321.42) |
| Sub 2-11 | m/z = 371.17 (C₂₈H₂₁N = 371.48) |
| Sub 2-13 | m/z = 321.15 (C₂₄H₁₉N = 321.42) |
| Sub 2-15 | m/z = 471.2 (C₃₆H₂₅N = 471.6) |
| Sub 2-17 | m/z = 335.17 (C₂₅H₂₁N = 335.45) |
| Sub 2-19 | m/z = 361.18 (C₂₇H₂₃N = 361.49) |
| Sub 2-21 | m/z = 513.25 (C₃₉H₃₁N = 513.68) |
| Sub 2-23 | m/z = 409.18 (C₃₁H₂₃N = 409.53) |
| Sub 2-25 | m/z = 485.21 (C₃₇H₂₇N = 485.63) |
| Sub 2-27 | m/z = 477.19 (C₃₅H₂₄FN = 477.58) |
| Sub 2-29 | m/z = 423.16 (C₃₁H₂₁NO = 423.52) |
| Sub 2-31 | m/z = 599.26 (C₄₆H₃₃N = 599.78) |
| Sub 2-33 | m/z = 407.17 (C₃₁H₂₁N = 407.52) |
| Sub 2-35 | m/z = 533.21 (C₄₁H₂₇N = 533.67) |
| Sub 2-37 | m/z = 335.13 (C₂₄H₁₇NO = 335.41) |
| Sub 2-39 | m/z = 259.1 (C₁₈H₁₃NO = 259.31) |
| Sub 2-41 | m/z = 335.13 (C₂₄H₁₇NO = 335.41) |
| Sub 2-43 | m/z = 309.12 (C₂₂H₁₅NO = 309.37) |
| Sub 2-45 | m/z = 385.15 (C₂₈H₁₉NO = 385.47) |
| Sub 2-47 | m/z = 411.16 (C₃₀H₂₁NO = 411.5) |
| Sub 2-49 | m/z = 425.18 (C₃₁H₂₃NO = 425.53) |
| Sub 2-51 | m/z = 275.08 (C₁₈H₁₃NS = 275.37) |
| Sub 1-53 | m/z = 351.11 (C₂₄H₁₇NS = 351.47) |
| Sub 1-55 | m/z = 275.08 (C₁₈H₁₃NS = 275.37) |
| Sub 1-57 | m/z = 325.09 (C₂₂H₁₅NS = 325.43) |
| Sub 1-59 | m/z = 504.17 (C₃₅H₂₄N₂S = 504.65) |
| Sub 1-61 | m/z = 478.15 (C₃₃H₂₂N₂S = 478.61) |
| Sub 1-63 | m/z = 516.17 (C₃₆H₂₄N₂S = 516.66) |
| Sub 1-65 | m/z = 334.15 (C₂₄H₁₈N₂ = 334.42) |
| Sub 1-67 | m/z = 410.18 (C₃₀H₂₂N₂ = 410.52) |
| Sub 1-69 | m/z = 486.21 (C₃₆H₂₆N₂ = 486.62) |
| Sub 1-71 | m/z = 384.16 (C₂₈H₂₀N₂ = 384.48) |
| Sub 1-73 | m/z = 245.12 (C₁₈H₁₅N = 245.33) |
| Sub 1-75 | m/z = 250.15 (C₁₈H₁₀D₅N = 250.36) |
| Sub 1-77 | m/z = 249.08 (C₁₆H₁₁NO₂ = 249.27) |
| Sub 1-79 | m/z = 225.06 (C₁₄H₁₁NS = 225.31) |
| Sub 1-81 | m/z = 270.12 (C₁₉H₁₄N₂ = 270.34) |
| Sub 1-83 | m/z = 445.14 (C₂₇H₁₉N₅S = 445.54) |
| Sub 2-2 | m/z = 219.1 (C₁₆H₁₃N = 219.29) |
| Sub 2-4 | m/z = 245.12 (C₁₈H₁₅N = 245.33) |
| Sub 2-6 | m/z = 269.12 (C₂₀H₁₅N = 269.35) |
| Sub 2-8 | m/z = 321.15 (C₂₄H₁₉N = 321.42) |
| Sub 2-10 | m/z = 345.15 (C₂₆H₁₉N = 345.45) |
| Sub 2-12 | m/z = 345.15 (C₂₆H₁₉N = 345.45) |
| Sub 2-14 | m/z = 397.18 (C₃₀H₂₃N = 397.52) |
| Sub 2-16 | m/z = 285.15 (C₂₁H₁₉N = 285.39) |
| Sub 2-18 | m/z = 361.18 (C₂₇H₂₃N = 361.49) |
| Sub 2-20 | m/z = 401.21 (C₃₀H₂₇N = 401.55) |
| Sub 2-22 | m/z = 335.17 (C₂₅H₂₁N = 335.45) |
| Sub 2-24 | m/z = 485.21 (C₃₇H₂₇N = 485.63) |
| Sub 2-26 | m/z = 459.2 (C₃₅H₂₅N = 459.59) |
| Sub 2-28 | m/z = 485.21 (C₃₇H₂₇N = 485.63) |
| Sub 2-30 | m/z = 483.2 (C₃₇H₂₅N = 483.61) |
| Sub 2-32 | m/z = 507.2 (C₃₉H₂₅N = 507.64) |
| Sub 2-34 | m/z = 458.18 (C₃₄H₂₂N₂ = 458.56) |
| Sub 2-36 | m/z = 259.1 (C₁₈H₁₃NO = 259.31) |
| Sub 2-38 | m/z = 335.13 (C₂₄H₁₇NO = 335.41) |
| Sub 2-40 | m/z = 335.13 (C₂₄H₁₇NO = 335.41) |
| Sub 2-42 | m/z = 309.12 (C₂₂H₁₅NO = 309.37) |
| Sub 2-44 | m/z = 385.15 (C₂₈H₁₉NO = 385.47) |
| Sub 2-46 | m/z = 411.16 (C₃₀H₂₁NO = 411.5) |
| Sub 2-48 | m/z = 335.13 (C₂₄H₁₇NO = 335.41) |
| Sub 2-50 | m/z = 412.16 (C₂₉H₂₀N₂O = 412.49) |
| Sub 2-52 | m/z = 275.08 (C₁₈H₁₃NS = 275.37) |
| Sub 1-54 | m/z = 351.11 (C₂₄H₁₇NS = 351.47) |
| Sub 1-56 | m/z = 325.09 (C₂₂H₁₅NS = 325.43) |
| Sub 1-58 | m/z = 325.09 (C₂₂H₁₅NS = 325.43) |
| Sub 1-60 | m/z = 431.13 (C₂₉H₂₁NOS = 431.55) |
| Sub 1-62 | m/z = 580.2 (C₄₁H₂₈N₂S = 580.75) |
| Sub 1-64 | m/z = 427.14 (C₃₀H₂₁NS = 427.57) |
| Sub 1-66 | m/z = 410.18 (C₃₀H₂₂N₂ = 410.52) |
| Sub 1-68 | m/z = 384.16 (C₂₈H₂₀N₂ = 384.48) |
| Sub 1-70 | m/z = 384.16 (C₂₈H₂₀N₂ = 384.48) |
| Sub 1-72 | m/z = 384.16 (C₂₈H₂₀N₂ = 384.48) |
| Sub 1-74 | m/z = 287.11 (C₂₀H₁₄FN = 287.34) |
| Sub 1-76 | m/z = 286.11 (C₁₉H₁₄N₂O = 286.33) |
| Sub 1-78 | m/z = 285.12 (C₂₀H₁₅NO = 285.35) |
| Sub 1-80 | m/z = 278.06 (C₁₅H₁₀N₄S = 278.33) |
| Sub 1-82 | m/z = 336.16 (C₂₄H₂₀N₂ = 336.44) |
| Sub 1-84 | m/z = 502.2 (C₃₆H₂₆N₂O = 502.62) |

### Synthesis Example of Final Compound

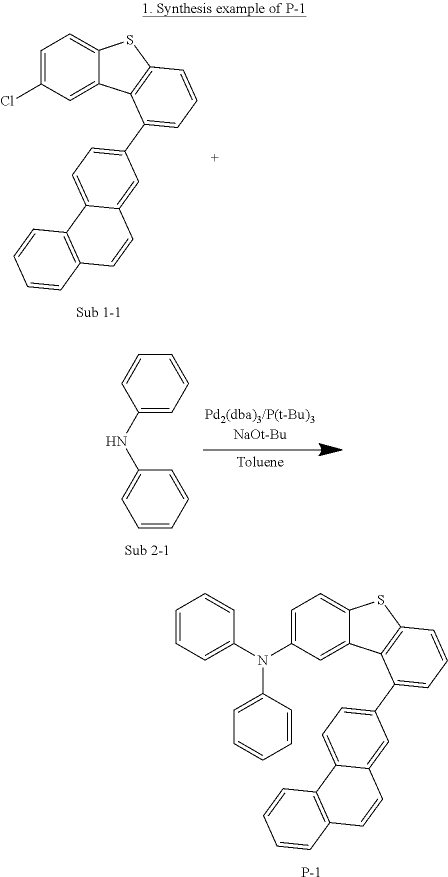

After dissolving Sub 1-1 (20 g, 50.6 mmol) in toluene (150 ml), Sub 2-1 (8.6 g, 50.6 mmol), Pd₂(dba)₃ (1.4 g, 1.5 mmol), 50% P(t-Bu)₃ (1.2 ml, 3.0 mmol) and NaOt-Bu (14.6 g, 151.9 mmol) were added thereto and the mixture was stirred at 130° C. When the reaction was completed, the reaction product was extracted with toluene and water. Then, an organic layer was dried over MgSO₄ and concentrated. Then, the concentrate was separated by a silica gel column and recrystallized to obtain 21.3 g (yield: 79.7%) of the product.

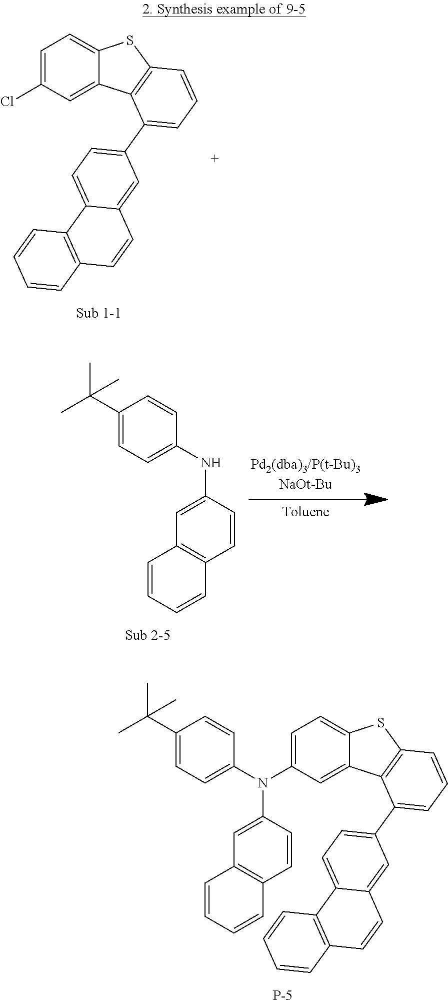

After dissolving Sub 1-1 (22 g, 55.7 mmol) in toluene (200 ml), Sub 2-5 (15.3 g, 55.7 mmol), Pd₂(dba)₃ (1.5 g, 1.7 mmol), 50% P(t-Bu)₃ (1.4 ml, 3.3 mmol) and NaOt-Bu (16.1 g, 167.1 mmol) were added thereto, and then the synthesis was carried out in the same manner as in the synthesis method of P-1 to obtain 24.7 g (yield: 70.0%) of the product.

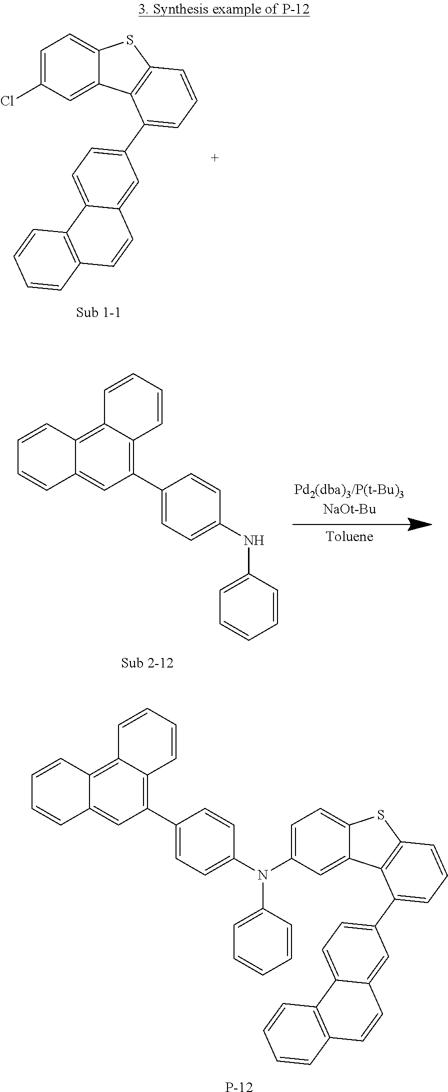

After dissolving Sub 1-1 (24 g, 60.8 mmol) in toluene (220 ml), Sub 2-12 (21.0 g, 60.8 mmol), Pd₂(dba)₃ (1.7 g, 1.8 mmol), 50% P(t-Bu)₃ (1.5 ml, 3.6 mmol) and NaOt-Bu (17.5 g, 182.3 mmol) were added thereto, and then the synthesis was carried out in the same manner as in the synthesis method of P-1 to obtain 28.1 g (yield: 72.9%) of the product.

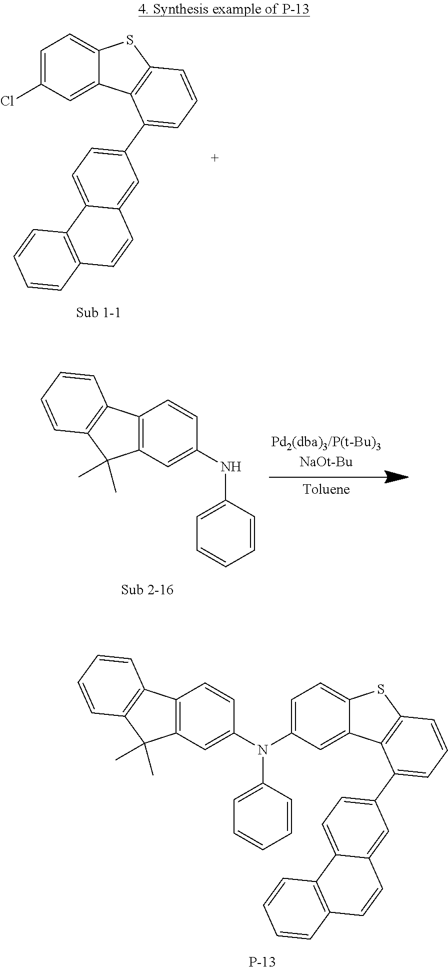

After dissolving Sub 1-1 (23 g, 58.2 mmol) in toluene (210 ml), Sub 2-16 (16.6 g, 58.2 mmol), Pd₂(dba)₃ (1.6 g, 1.7 mmol), 50% P(t-Bu)₃ (1.4 ml, 3.5 mmol) and NaOt-Bu (16.8 g, 174.7 mmol) were added thereto, and then the synthesis was carried out in the same manner as in the synthesis method of P-1 to obtain 26.5 g (yield: 71.8%) of the product.

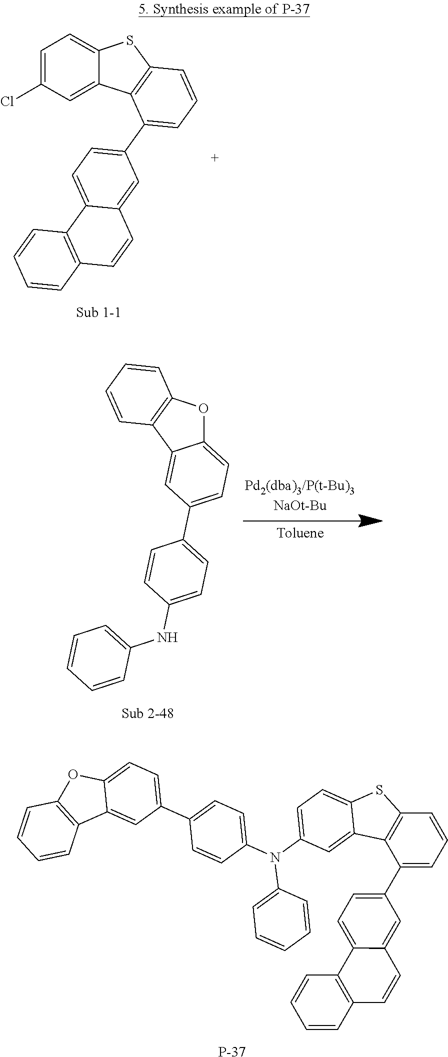

After dissolving Sub 1-1 (30 g, 76.0 mmol) in toluene (300 ml), Sub 2-48 (25.5 g, 76.0 mmol), Pd₂(dba)₃ (2.1 g, 2.3 mmol), 50% P(t-Bu)₃ (1.8 ml, 4.6 mmol) and NaOt-Bu (21.9 g, 227.9 mmol) were added thereto, and then the synthesis was carried out in the same manner as in the synthesis method of P-1 to obtain 33.5 g (yield: 69.6%) of the product.

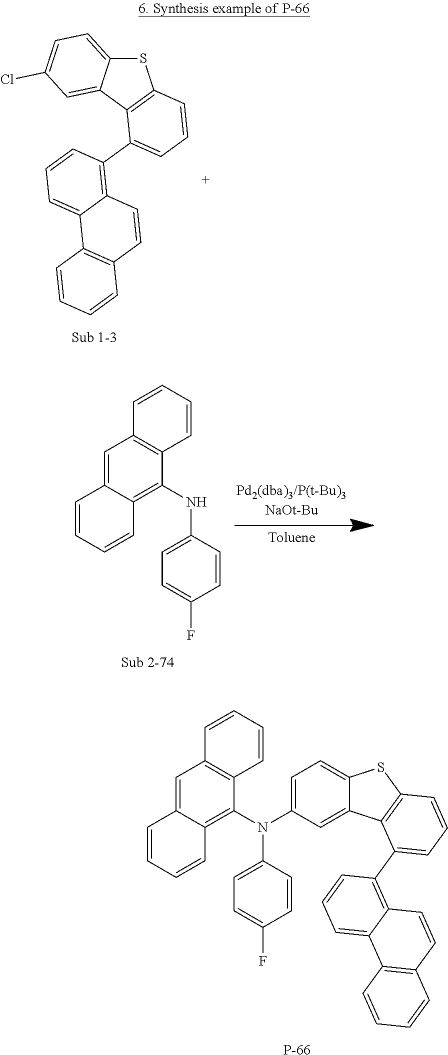

After dissolving Sub 1-3 (33 g, 83.6 mmol) in toluene (350 ml), Sub 2-74 (24.0 g, 83.6 mmol), Pd₂(dba)₃ (2.3 g, 2.5 mmol), 50% P(t-Bu)₃ (2.0 ml, 5.0 mmol) and NaOt-Bu (24.1 g, 250.7 mmol) were added thereto, and then the synthesis was carried out in the same manner as in the synthesis method of P-1 to obtain 36.2 g (yield: 67.1%) of the product.

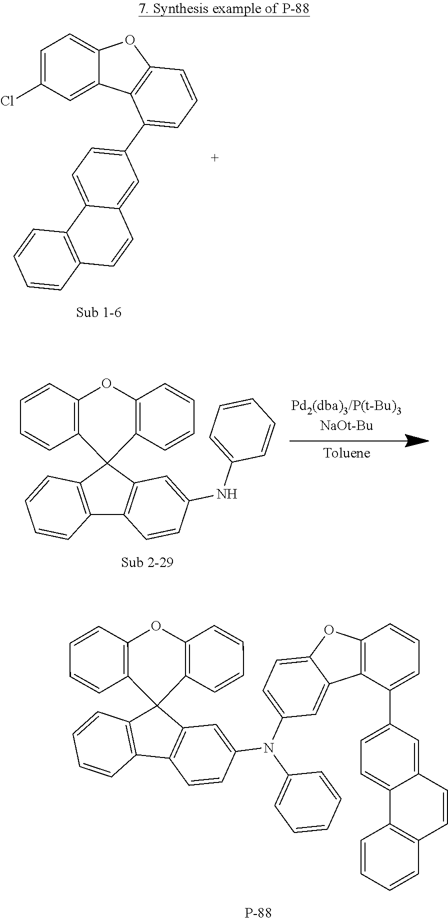

After dissolving Sub 1-6 (29 g, 76.5 mmol) in toluene (290 ml), Sub 2-29 (32.4 g, 76.5 mmol), Pd₂(dba)₃ (2.1 g, 2.3 mmol), 50% P(t-Bu)₃ (1.9 ml, 4.6 mmol) and NaOt-Bu (22.0 g, 229.6 mmol) were added thereto, and then the synthesis was carried out in the same manner as in the synthesis method of P-1 to obtain 40.2 g (yield: 68.6%) of the product.

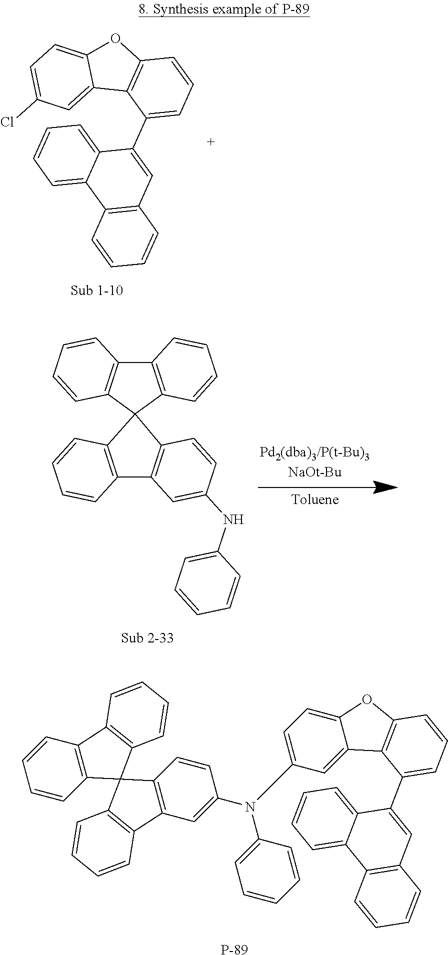

After dissolving Sub 1-10 (25 g, 66.0 mol) in toluene (250 ml), Sub 2-33 (26.9 g, 66.0 mmol), Pd₂(dba)₃ (1.8 g, 2.0 mmol), 50% P(t-Bu)₃ (1.6 ml, 4.0 mmol) and NaOt-Bu (19.0 g, 198.0 mmol) were added thereto, and then the synthesis was carried out in the same manner as in the synthesis method of P-1 to obtain 37 g (yield: 75.0%) of the product.

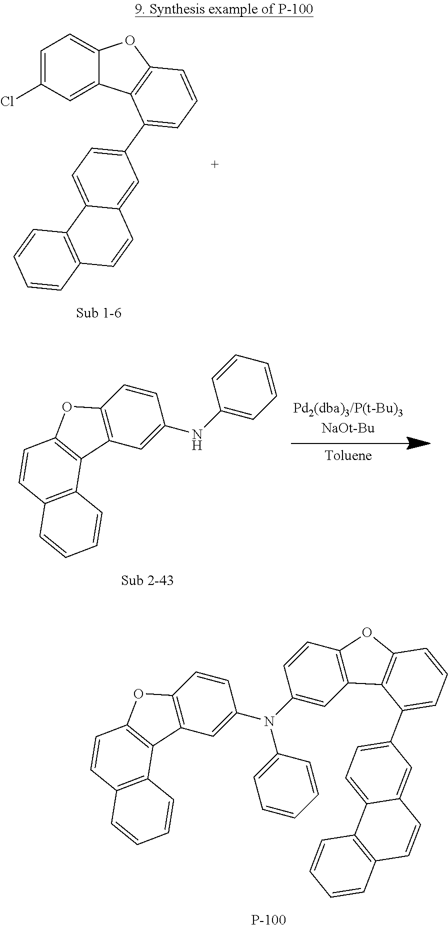 [SMILES: Clc1ccc2oc3cccc(-c4ccc5c(ccc6ccccc65)c4)c3c2c1 ; c1ccc(Nc2ccc3oc4ccc5ccccc5c4c3c2)cc1 ; c1ccc(N(c2ccc3oc4cccc(-c5ccc6c(ccc7ccccc76)c5)c4c3c2)c2ccc3oc4ccc5ccccc5c4c3c2)cc1]

After dissolving Sub 1-6 (34 g, 89.7 mmol) in toluene (340 ml), Sub 2-43 (27.8 g, 89.7 mmol), Pd₂(dba)₃ (2.5 g, 2.7 mmol), 50% P(t-Bu)₃ (2.2 ml, 5.4 mmol) and NaOt-Bu (25.9 g, 269.2 mmol) were added thereto, and then the synthesis was carried out in the same manner as in the synthesis method of P-1 to obtain 41.2 g (yield: 70.4%) of the product.

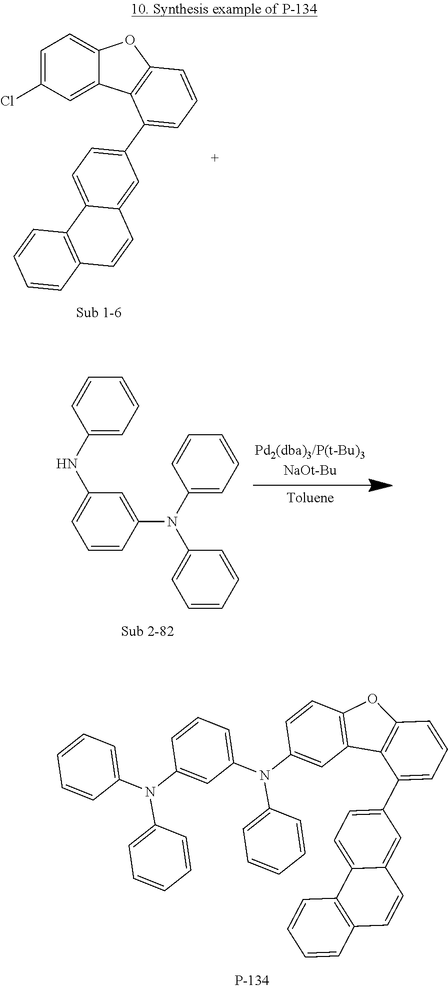 [SMILES: Clc1ccc2oc3cccc(-c4ccc5c(ccc6ccccc65)c4)c3c2c1 ; c1ccc(Nc2cccc(N(c3ccccc3)c3ccccc3)c2)cc1 ; c1ccc(N(c2ccccc2)c2cccc(N(c3ccccc3)c3ccc4oc5cccc(-c6ccc7c(ccc8ccccc87)c6)c5c4c3)c2)cc1]

After dissolving Sub 1-6 (27 g, 71.2 mmol) in toluene (270 ml), Sub 2-82 (24.0 g, 71.2 mmol), Pd₂(dba)₃ (2.0 g, 2.1 mmol), 50% P(t-Bu)₃ (1.7 ml, 4.3 mmol) and NaOt-Bu (20.5 g, 213.8 mmol) were added thereto, and then the synthesis was carried out in the same manner as in the synthesis method of P-1 to obtain 38 g (yield: 78.5%) of the product.

FD-MS values of the compounds of the present invention synthesized by the above synthesis method are shown in Table 3 below.

TABLE 3

| Compound | FD-MS |
|---|---|
| P-1 | m/z = 527.17 (C₃₈H25NS = 527.69) |
| P-3 | m/z = 603.20 (C₄₄H29NS = 603.78) |
| P-5 | m/z = 633.25 (C₄₆H₃₅NS = 633.85) |
| P-7 | m/z = 627.20 (C₄₆H₂₉NS = 627.81) |
| P-9 | m/z = 679.23 (C₅₀H₃₃NS = 679.88) |
| P-11 | m/z = 729.25 (C₅₄H₃₅NS = 729.94) |
| P-13 | m/z = 643.23 (C₄₇H₃₃NS = 643.85) |
| P-15 | m/z = 719.26 (C₅₃H₃₇NS = 719.95) |
| P-17 | m/z = 767.26 (C₅₇H₃₇NS = 767.99) |
| P-19 | m/z = 843.30 (C₆₃H₄₁NS = 844.09) |
| P-21 | m/z = 781.24 (C₅₇H₃₅NOS = 781.97) |
| P-23 | m/z = 957.34 (C₇₂H₄₇NS = 958.24) |
| P-25 | m/z = 617.18 (C₄₄H₂₇NOS = 617.77) |
| P-27 | m/z = 693.21 (C₅₀H₃₁NOS = 693.86) |
| P-29 | m/z = 693.21 (C₅₀H₃₁NOS = 693.86) |
| P-31 | m/z = 667.20 (C₄₈H₂₉NOS = 667.83) |
| P-33 | m/z = 921.30 (C₆₃H₃₅D₅N₄S₂ = 922.19) |
| P-35 | m/z = 769.24 (C₅₆H₃₅NOS = 769.96) |
| P-37 | m/z = 693.21 (C₅₀H₃₁NOS = 693.86) |
| P-39 | m/z = 769.24 (C₅₆H₃₅NOS = 769.96) |
| P-41 | m/z = 783.26 (C₅₇H₃₇NOS = 783.99) |
| P-43 | m/z = 633.16 (C₄₄H₂₇NS₂ = 633.83) |
| P-45 | m/z = 709.19 (C₅₀H₃₁NS₂ = 709.93) |
| P-47 | m/z = 633.16 (C₄₄H₂₇NS₂ = 633.83) |
| P-49 | m/z = 683.17 (C₄₈H₂₉NS₂ = 683.89) |
| P-51 | m/z = 862.25 (C₆₁H₃₈N₂S₂ = 863.11) |
| P-53 | m/z = 692.23 (C₅₀H₃₂N₂S = 692.88) |
| P-55 | m/z = 768.26 (C₅₆H₃₆N2S = 768.98) |
| P-57 | m/z = 692.23 (C₅₀H₃₂N₂S = 692.88) |
| P-59 | m/z = 768.26 (C₅₆H₃₆N₂S = 768.98) |
| P-61 | m/z = 974.26 (C₅₀H₃₂N₂S = 975.26) |
| P-63 | m/z = 742.24 (C₅₄H₃₄N₂S = 742.94) |
| P-65 | m/z = 603.20 (C44H29NS = 603.78) |
| P-67 | m/z = 608.23 (C₄₄H₂₄D₅NS = 608.81) |
| P-69 | m/z = 511.19 (C38H25NO = 511.62) |
| P-71 | m/z = 587.22 (C44H29NO = 587.72) |
| P-73 | m/z = 617.27 (C₄₆H₃₅NO = 617.79) |
| P-75 | m/z = 611.22 (C₄₆H₂₉NO = 611.74) |
| P-77 | m/z = 663.26 (C50H33NO = 663.82) |
| P-79 | m/z = 739.29 (C₅₆H₃₇NO = 739.92) |
| P-81 | m/z = 627.26 (C₄₇H₃₃NO = 627.79) |
| P-83 | m/z = 1082.24 (C₆₉H₄₂N₆S₄ = 1083.38) |
| P-85 | m/z = 751.29 (C₅₇H₃₇NO = 751.93) |
| P-87 | m/z = 827.32 (C₆₃H₄₁NO = 828.03) |
| P-89 | m/z = 749.27 (C₅₇H35NO = 749.91) |
| P-91 | m/z = 800.28 (C₆₀H₃₆N₂O = 800.96) |
| P-93 | m/z = 601.20 (C₄₄H₂₇NO₂ = 601.71) |
| P-95 | m/z = 677.24 (C₅₀H₃₁NO₂ = 601.71) |
| P-97 | m/z = 677.24 (C₅₀H₃₁NO₂ = 677.80) |
| P-99 | m/z = 651.22 (C₄₈H₂₉NO₂ = 651.77) |
| P-101 | m/z = 727.25 (C₅₄H₃₃NO₂ = 727.86) |
| P-103 | m/z = 753.27 (C₅₆H₃₅NO₂ = 753.90) |
| P-105 | m/z = 677.24 (C₅₀H₃₁NO₂ = 677.80) |
| P-107 | m/z = 753.27 (C₅₆H₃₅NO₂ = 753.90) |
| P-109 | m/z = 1049.16 (C₆₃H₃₅N₇S₅ = 1050.32) |
| P-111 | m/z = 617.18 (C₄₄H₂₇NOS = 617.77) |
| P-113 | m/z = 693.21 (C₅₀H₃₁NOS = 693.86) |
| P-115 | m/z = 617.18 (C₄₄H₂₇NOS = 617.77) |
| P-117 | m/z = 667.20 (C₄₈H₂₉NOS = 667.83) |
| P-119 | m/z = 846.27 (C₆₁H₃₈N₂OS = 847.05) |
| P-121 | m/z = 676.25 (C₅₀H₃₂N₂O = 676.82) |
| P-123 | m/z = 752.28 (C₅₆H₃₆N₂O = 752.92) |
| P-125 | m/z = 828.31 (C₆₂H₄₀N₂O = 829.02) |
| P-127 | m/z = 726.27 (C₅₄H₃₄N₂O = 726.88) |
| P-129 | m/z = 591.18 (C₄₂H₂₅NO₃ = 591.67) |
| P-131 | m/z = 567.17 (C₄₀H₂₅NOS = 567.712) |
| P-133 | m/z = 612.22 (C₄₅H₂₈N2O = 612.73) |
| P-135 | m/z = 844.31 (C₆₂H₄₀N₂O₂ = 845.01) |
| P-137 | m/z = 611.22 (C₄₆H₂₉NO = 611.74) |
| P-139 | m/z = 637.24 (C₄₈H₃₁NO = 637.78) |
| P-141 | m/z = 678.23 (C₄₉H₃₀N₂O₂ = 678.79) |
| P-143 | m/z = 627.2 (C₄₆H₂₉NS = 627.81) |
| P-2 | m/z = 577.19 (C₄₂H₂₇NS = 577.75) |
| P-4 | m/z = 603.20 (C₄₄H₂₉NS = 603.78) |
| P-6 | m/z = 627.20 (C₄₆H₂₉NS = 627.81) |
| P-8 | m/z = 679.23 (C₅₀H₃₃NS = 679.88) |
| P-10 | m/z = 703.23 (C₅₂H₃₃NS = 703.90) |
| P-12 | m/z = 703.23 (C₅₂H₃₃NS = 703.90) |
| P-14 | m/z = 693.25 (C₅₁H₃₅NS = 693.91) |
| P-16 | m/z = 719.26 (C₅₃H₃₇NS = 719.95) |
| P-18 | m/z = 843.30 (C₆₃H₄₁NS = 844.09) |
| P-20 | m/z = 843.30 (C₆₃H₄₁NS = 844.09) |
| P-22 | m/z = 841.28 (C₆₃H₃₉NS = 842.07) |
| P-24 | m/z = 865.28 (C₆₅H₃₉NS = 866.09) |
| P-26 | m/z = 693.21 (C₅₀H₃₁NOS = 693.86) |
| P-28 | m/z = 617.18 (C₄₄H₂₇NOS = 617.77) |
| P-30 | m/z = 693.21 (C₅₀H₃₁NOS = 693.86) |
| P-32 | m/z = 667.20 (C₄₈H₃₀N₂S₃ = 667.83) |
| P-34 | m/z = 743.23 (C₅₄H₃₃NOS = 743.92) |
| P-36 | m/z = 769.24 (C₅₆H₃₅NOS = 769.96) |
| P-38 | m/z = 743.23 (C₅₄H₃₃NOS = 743.92) |
| P-40 | m/z = 769.24 (C₅₆H₃₅NOS = 769.96) |
| P-42 | m/z = 770.24 (C₅₅H₃₄N₂OS = 770.95) |
| P-44 | m/z = 633.16 (C₄₄H₂₇NS₂ = 633.83) |
| P-46 | m/z = 709.19 (C₅₀H₃₁NS₂ = 709.93) |
| P-48 | m/z = 683.17 (C₄₈H₂₉NS₂ = 683.89) |
| P-50 | m/z = 683.17C₄₈H₂₉NS = 683.89) |
| P-52 | m/z = 789.22 (C₅₅H₃₅NOS₂ = 790.01) |
| P-54 | m/z = 768.26 (C₅₆H₃₆N₂S = 768.98) |
| P-56 | m/z = 742.24 (C₅₄H₃₄N₂S = 742.94) |
| P-58 | m/z = 768.26 (C₅₆H₃₆N₂S = 768.98) |
| P-60 | m/z = 742.24 (C₅₄H₃₄N₂S = 742.94) |
| P-62 | m/z = 742.24 (C₅₄H₃₄N₂S = 742.94) |
| P-64 | m/z = 742.24 (C₅₄H₃₄N₂S = 742.94) |
| P-66 | m/z = 645.19 (C₄₆H₂₈FNS = 645.80) |
| P-68 | m/z = 644.19 (C₄₅H₂₈N₂OS = 644.79) |
| P-70 | m/z = 561.21 (C₄₂H₂₇NO = 561.68) |
| P-72 | m/z = 587.22 (C₄₄H₂₉NO = 587.72) |
| P-74 | m/z = 611.22 (C₄₆H₂₉NO = 611.74) |
| P-76 | m/z = 663.26 (C₅₀H₃₃NO = 663.82) |
| P-78 | m/z = 663.26 (C₅₀H₃₃NO = 663.82) |
| P-80 | m/z = 889.33 (C₆₈H₄₃NO = 890.10) |
| P-82 | m/z = 743.32 (C₅₆H₄₁NO = 743.95) |
| P-84 | m/z = 677.27 (C₅₁H₃₅NO = 677.85) |
| P-86 | m/z = 819.29 (C₆₁H₃₈FNO = 819.98) |
| P-88 | m/z = 765.27 (C₅₇H₃₅NO₂ = 765.91) |
| P-90 | m/z = 825.30 (C₆₃H₃₉NO = 826.01) |
| P-92 | m/z = 875.32 (C₆₇H₄₁NO = 876.07) |
| P-94 | m/z = 677.24 (C₅₀H₃₁NO₂ = 677.80) |
| P-96 | m/z = 601.20 (C₄₄H₂₇NO₂ = 992.21) |
| P-98 | m/z = 677.24 (C₅₀H₃₁NO₂ = 677.80) |
| P-100 | m/z = 651.22 (C₄₈H₂₉NO₂ = 651.77) |
| P-102 | m/z = 727.25 (C₅₄H₃₃NO₂ = 727.86) |
| P-104 | m/z = 753.27 (C₅₆H₃₅NO₂) = 753.90) |
| P-106 | m/z = 727.25 (C₅₄H₃₃NO₂ = 727.86) |
| P-108 | m/z = 1152.31 (C₅₆H₃₅NO₂ = 1153.45) |
| P-110 | m/z = 754.26 (C₅₅H₃₄N₂O₂ = 754.89) |
| P-112 | m/z = 617.18 (C₄₄H₂₇NOS = 617.77) |
| P-114 | m/z = 693.21 (C₅₀H₃₁NOS = 693.86) |
| P-116 | m/z = 667.20 (C₄₈H₂₉NOS = 667.83) |
| P-118 | m/z = 667.20 (C₄₈H₂₉NOS = 667.83) |
| P-120 | m/z = 1152.31 (C₇₇H₄₈N₆S₃ = 1153.45) |
| P-122 | m/z = 752.28 (C₅₆H₃₆N₂O = 752.92) |
| P-124 | m/z = 726.27 (C₅₄H₃₄N₂O = 726.88) |
| P-126 | m/z = 726.27 (C₅₄H₃₄N₂O = 726.88) |
| P-128 | m/z = 726.27 (C₅₄H₃₄N₂O = 726.88) |
| P-130 | m/z = 627.22 (C₄₆H₂₉NO₂ = 627.74) |
| P-132 | m/z = 1152.31 (C₇₇H₄₈N₆S₃ = 1153.45) |
| P-134 | m/z = 678.27 (C₅₀H₃₄N₂O = 678.84) |
| P-136 | m/z = 787.24 (C₅₃H₃₃N₅OS = 787.94) |
| P-138 | m/z = 627.2 (C₄₆H₂₉NS = 627.81) |
| P-140 | m/z = 744.22 (C₅₃H₃₂N₂OS = 744.91) |
| P-142 | m/z = 637.24 (C₄₈H₃₁NO = 637.78) |
| P-144 | m/z = 677.22 (C₅₀H₃₁NS = 677.87) |

### Fabrication and Evaluation of Organic Electric Element

### [Test Example 1] Red Organic Electroluminescent Element (Emission-Auxiliary Layer)

After vacuum-depositing N1-(naphthalen-2-yl)-N4,N4-bis(4-(naphthalen-2-yl(phenyl)amino)phenyl)-N1-phenylbenzene-1,4-diamine (hereinafter abbreviated as “2-TNATA”) on an ITO layer (anode) formed on a glass substrate to form a hole injection layer of 60 nm thickness, a hole transport layer of 60 nm thickness was formed by vacuum-depositing N,N′-bis(1-naphthalenyl)-N,N′-bis-phenyl-(1,1′-biphenyl)-4,4′-diamine (hereinafter abbreviated as “NPB”) on the hole injection layer.

Subsequently, an emission-auxiliary layer of 20 nm thickness was formed by vacuum-depositing the compound P-3 of the present invention on the hole transport layer and 4,4′-N,N′-dicarbazole-biphenyl (hereinafter abbreviated as “CBP”) as a host material and bis-(1-phenyl isoquinolyl)iridium(III)acetylacetonate (hereinafter abbreviated as “(piq)₂Ir(acac)”) as a dopant material in a weight ratio of 95:5 were deposited on the emission-auxiliary layer to form a light emitting layer of 30 nm thickness.

Next, (1,1′-bisphenyl-4-olato)bis(2-methyl-8-quinolinolato)aluminum (hereinafter abbreviated as “BAlq”) was vacuum-deposited to form a hole blocking layer of 10 nm thickness on the light emitting layer, and tris-(8-hydroxyquinoline)aluminum (hereinafter abbreviated as “Alq₃”) was vacuum-deposited to form a an electron transport layer of 40 nm thickness on the hole blocking layer.

Thereafter, LiF was deposited to form an electron injection layer of 0.2 nm thickness on the electron transport layer, and then Al was deposited to form a cathode of 150 nm thickness on the electron injection layer.

### [Test Example 2] to [Test Example 9]

The organic electroluminescent elements were fabricated in the same manner as described in Example 1 except that the compounds of the present invention described in the following Table 4, instead of compound P-3 of the present invention, were used as material of an emission-auxiliary layer.

### Comparative Example 1

The organic electroluminescent element was fabricated in the same manner as in Example 1, except that an emission-auxiliary layer was not formed.

### [Comparative Example 2] to [Comparative Example 4]

The organic electroluminescent elements were fabricated in the same manner as described in Example 1 except that one of Comparative Compounds A to C, instead of compound P-3 of the present invention, was used as material of an emission-auxiliary layer.

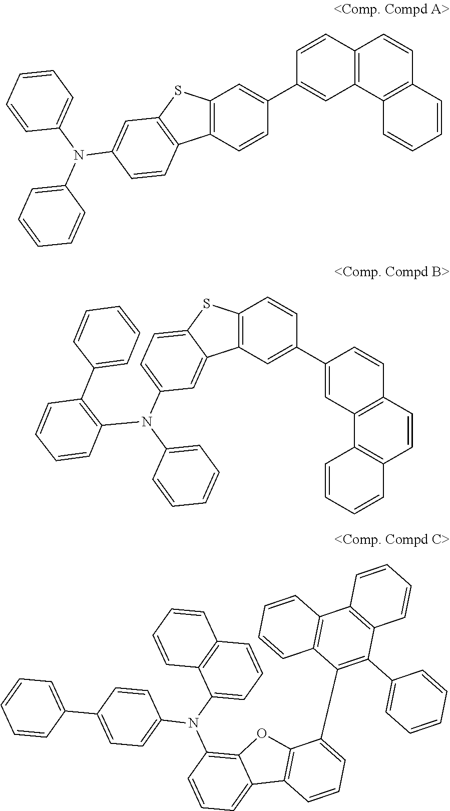 [SMILES: c1ccc(N(c2ccccc2)c2ccc3c(c2)sc2cc(-c4ccc5ccc6ccccc6c5c4)ccc23)cc1 ; c1ccc(-c2ccccc2N(c2ccccc2)c2ccc3sc4ccc(-c5ccc6ccc7ccccc7c6c5)cc4c3c2)cc1 ; c1ccc(-c2ccc(N(c3cccc4ccccc34)c3cccc4c3oc3c(-c5c(-c6ccccc6)c6ccccc6c6ccccc56)cccc34)cc2)cc1]

A forward bias DC voltage was applied to the electroluminescent elements manufactured in Test Examples 1 to 9 and Comparative Examples 1 to 4 and electroluminescence (EL) characteristics were measured with Photo Research's PR-650 and lifetime (T95) was measured with a lifetime measuring device manufactured by Mc Science company at 2500 cd/m² standard luminance. The measurement results are shown in Table 4 below.

TABLE 4

| | | | Current | | | |
|---|---|---|---|---|---|---|
| | | Voltage | Density | Brightness | Efficiency | Lifetime |
| | Compound | (V) | (mA/cm²) | (cd/m²) | (cd/A) | T(95) |
| comp. Ex(1) | — | 6.9 | 39.1 | 2500.0 | 6.4 | 60.1 |
| comp. Ex(2) | Comp. compd A | 6.2 | 21.7 | 2500.0 | 11.5 | 81.5 |
| comp. Ex(3) | Comp. compd B | 6.4 | 22.3 | 2500.0 | 11.2 | 76.8 |
| comp. Ex(4) | Comp. compd C | 6.7 | 20.3 | 2500.0 | 12.3 | 92.8 |
| Test Ex. (1) | P-3 | 5.1 | 8.4 | 2500.0 | 29.8 | 126.4 |
| Test Ex. (2) | P-12 | 5.0 | 8.6 | 2500.0 | 29.1 | 124.4 |
| Test Ex. (3) | P-15 | 4.9 | 8.2 | 2500.0 | 30.5 | 168.5 |
| Test Ex. (4) | P-30 | 4.8 | 8.9 | 2500.0 | 28.0 | 152.1 |
| Test Ex. (5) | P-48 | 4.8 | 9.8 | 2500.0 | 25.4 | 118.7 |
| Test Ex. (6) | P-76 | 5.0 | 8.7 | 2500.0 | 28.7 | 153.7 |
| Test Ex. (7) | P_88 | 5.2 | 9.3 | 2500.0 | 26.9 | 128.8 |
| Test Ex. (8) | P-95 | 4.9 | 9.2 | 2500.0 | 27.2 | 117.2 |
| Test Ex. (9) | P-121 | 4.9 | 9.1 | 2500.0 | 27.6 | 123.9 |

Referring to Table 4 above, when the compound of the present invention is used as an emission auxiliary layer material, the driving voltage of the organic electroluminescence element is significantly lower and luminous efficiency and lifespan can be significantly improved, compared to the comparative examples without forming an emission auxiliary layer or using one of Comparative Compounds A to Comparative Compounds C.

Comparing Comparative Compounds A to C with the compound of the present invention, there are differences in the substitution positions of phenanthryl substituted in dibenzothiophene or dibenzofuran and the substitution positions of the amine group. The compounds of the present invention differ in the positions where the phenanthryl group and amine group are substituted on each benzene ring based on X in Formula 1, that is, they are substituted in asymmetric positions for each other. On the other hand, in the case of comparative compounds A to C, the phenanthryl and amine groups are substituted at the same position of each benzene ring based on S or O, that is, they are substituted in symmetric positions for each other. That is, the compound of the present invention has a phenanthryl group substituted at 1-position and an amine group substituted at 7-position of dibenzothiophene or dibenzofuran. On the other hand, comparative compound A has a phenanthryl group substituted at 3-position and an amine group substituted at 6-position, comparative compound B has a phenanthryl group substituted at 2-position and an amine group substituted at 7-position, and comparative compound C has a phenanthryl group substituted at 4-position and an amine group substituted at 5-position.

When an emission layer is formed with compound in which each substituent is substituted in a symmetrical position for each other, such as Comparative Compounds A to C, the overall material is not arranged evenly, thereby slowing down HOD (Hole Only Device) and becoming chemically unstable. As a result, it appears that when an emission auxiliary layer is formed with such a compound, the lifespan of the element is affected and the lifespan is reduced compared to the compound of the present invention.

In addition, since the HOMO or LUMO energy level of the compound of the present invention has an appropriate value between the hole transport layer and the light emitting layer, when the compound of the present invention is used as an emission auxiliary layer material, holes and electrons maintain charge balance and light is emitted from within a light emitting layer rather than at the hole transport layer interface, and thus it appears that efficiency and lifespan are maximized.

Comparing Test Examples 1 to 9 of the present invention, in a 3-condensed ring containing X, in the case of a dibenzothiophene core where X is S, when the compound in which the dibenzothiophene core has amine group substituted with a 3-condensed ring having different heteroatom is used as an emission auxiliary layer material (Examples 3 and 4), the lifespan is significantly improved while the driving voltage is lowered. In the case of a dibenzofuran core where X is O, when the compound in which the dibenzofuran core has amine group substituted with aryl group is used as an emission auxiliary layer material (Examples 6), the lifespan is significantly improved. From these results, it can be seen that the driving voltage or lifespan may vary depending on the type of heteroatom in the core and substituent substituted for the amine group.

### [Test Example 10] Red Organic Electroluminescent Element (Phosphorescent Host)

2-TNATA film was vacuum deposited on the ITO layer (anode) formed on the glass substrate to form a hole injection layer with a thickness of 60 nm, and then NPB was vacuum-deposited to form a hole transport layer with a thickness of 60 nm. Thereafter, compound DSNL1 (a first compound) and compound P-1 (a second compound) of the present invention were mixed at a ratio of 5:5, the mixture as a host material and (piq)₂Ir(acac) as a dopant in a weight ratio of 95:5 were deposited on the hole transport layer to form a light emitting layer of 30 nm thickness.

Next, BAlq was vacuum-deposited to form a hole blocking layer of 10 nm thickness on the light emitting layer, and Alq₃ was vacuum-deposited to form a an electron transport layer of 40 nm thickness on the hole blocking layer. Thereafter, LiF was deposited to form an electron injection layer of 0.2 nm thickness on the electron transport layer, and then Al was deposited to form a cathode of 150 nm thickness on the electron injection layer.

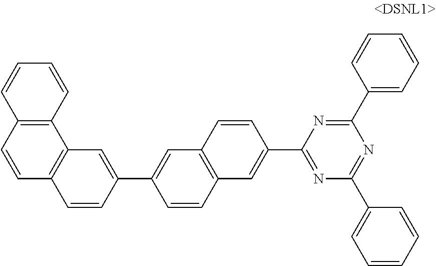 [SMILES: c1ccc(-c2nc(-c3ccccc3)nc(-c3ccc4cc(-c5ccc6ccc7ccccc7c6c5)ccc4c3)n2)cc1]

### [Test Example 11] to [Test Example 16]

The organic electroluminescent elements were fabricated in the same manner as described in Example 10 except that compound of the present invention described in the following Table 5, instead of compound P-1, was used as a second compound among the host materials of a light emitting layer.

### [Comparative Example 5] to [Comparative Example 7]

The organic electroluminescent elements were fabricated in the same manner as described in Example 10 except that one of Comparative Compounds A to C, instead of compound P-1, was used as a second compound among the host materials of a light emitting layer.

A forward bias DC voltage was applied to the electroluminescent elements manufactured in Test Examples 10 to 16 and Comparative Examples 5 to 7 and electroluminescence (EL) characteristics were measured with Photo Research's PR-650 and lifetime (T95) was measured with a lifetime measuring device manufactured by Mc Science company at 2500 cd/m² standard luminance. The measurement results are shown in Table 5 below.

TABLE 5

| | | | | Current | | | |
|---|---|---|---|---|---|---|---|
| | | | Voltage | Density | Brightness | Efficiency | Lifetime |
| | Compound 1 | Compound 2 | (V) | (mA/cm²) | (cd/m²) | (cd/A) | T(95) |
| comp. Ex(5) | DSNL1 | Comp. compd A | 5.1 | 11.7 | 2500 | 21.4 | 90.4 |
| comp. Ex(6) | | Comp. compd B | 5.2 | 11.3 | 2500 | 22.1 | 88.7 |
| comp. Ex(7) | | Comp. compd C | 6 | 10.4 | 2500 | 24.1 | 98.2 |
| Test Ex. (10) | | P-1 | 4.6 | 7.5 | 2500 | 33.3 | 141.1 |
| Test Ex. (11) | | P-9 | 4.5 | 7.3 | 2500 | 34.1 | 138.7 |
| Test Ex. (12) | | P-34 | 4.3 | 8.1 | 2500 | 30.8 | 132.6 |
| Test Ex. (13) | | P-37 | 4.4 | 8.0 | 2500 | 31.2 | 135.7 |
| Test Ex. (14) | | P-67 | 4.7 | 6.8 | 2500 | 36.8 | 143.5 |
| Test Ex. (15) | | P-90 | 4.5 | 6.6 | 2500 | 37.7 | 136.4 |
| Test Ex. (16) | | P-118 | 4.3 | 8.2 | 2500 | 30.5 | 131.0 |

Referring to Table 5 above, it can be seen that the driving voltage, efficiency and lifespan are significantly improved when using a mixture of DSNL1 and the compound of the present invention as a host, compared to when using a mixture of DSNL1 and one of the comparative compounds A to C.

As already explained, the compound of the present invention in which the phenanthryl group and amine group are bonded in asymmetric positions for each other based on X is different from comparative compounds in which they are bonded in symmetric positions for each other. The energy level of the compound and the HOD value vary depending on the symmetry of these binding sites. When the phenanthryl group and the amine group are bonded in an asymmetric position for each other, as in the compound of the present invention, the distance between the compounds becomes closer, and the hole characteristics are strengthened, resulting in a fast HOD value and excellent hole mobility. That is, in the case of the compound of the present invention, by substituting a phenanthryl group at 1-position of the core of dibenzothiophene or dibenzofuran, the distance between compounds is reduced by their interaction. This has the same effect in terms of the distance between compounds even when DSNL1, a different compound, is mixed.

In addition, because the compound of the present invention of Formula 1 contains S or O elements in the core, it exhibits a high refractive index and high Tg value, and has HOMO and LUMO levels that maximize the energy transfer effect. As a result, it appears that luminous efficiency is greatly improved and lifespan is also significantly improved due to high thermal stability.

Although the exemplary embodiments of the present invention have been described for illustrative purposes, those skilled in the art to which the present invention pertains will be capable of various modifications without departing from the essential characteristics of the present invention. Therefore, the embodiment disclosed herein is intended to illustrate the present invention rather than to limit the present invention and the scope of the present invention is not limited by the embodiments. The scope of the present invention shall be construed on the basis of the accompanying claims, and it shall be construed that all of the technical ideas included within the scope equivalent to the claims belong to the present invention.

1. A compound of Formula 1: 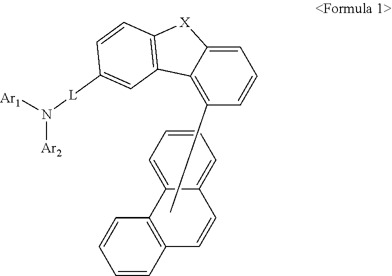
wherein:
X is O or S,
Ar₁ and Ar₂ are each independently selected from the group consisting of a C₆-C₆₀ aryl group, a fluorenyl group, a C₂-C₆₀ heterocyclic group containing at least one heteroatom of O, N, S, Si and P, and a C₃-C₆₀ aliphatic ring group,
L is selected from the group consisting of a single bond, a C₆-C₆₀ arylene group, a fluorenylene group, a C₂-C₆₀ heterocyclic group containing at least one heteroatom of O, N, S, Si and P and a C₃-C₆₀ aliphatic ring group,
the aryl group, the arylene group, the fluorenyl group, the fluorenylene group, the heterocyclic group, and the aliphatic ring group may be each optionally substituted with one or more substituents selected from the group consisting of deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group containing at least one heteroatom of O, N, S, Si and P, a C₃-C₃₀ aliphatic ring group and -L′-N(Rₐ)(R_{b}),
L′ is selected from the group consisting of a single bond, a C₆-C₃₀ arylene group, a fluorenylene group, a C₂-C₃₀ heterocyclic group containing at least one heteroatom of O, N, S, Si and P and a C₃-C₃₀ aliphatic ring group, and
Rₐ and R_{b} are each independently selected from the group consisting of a C₆-C₃₀ aryl group, a fluorenyl group, a C₂-C₃₀ heterocyclic group containing at least one heteroatom of O, N, S, Si and P, and a C₃-C₃₀ aliphatic ring group.

2. The compound of claim 1, wherein Formula 1 is represented by one of Formula 1-1 to Formula 1-5: 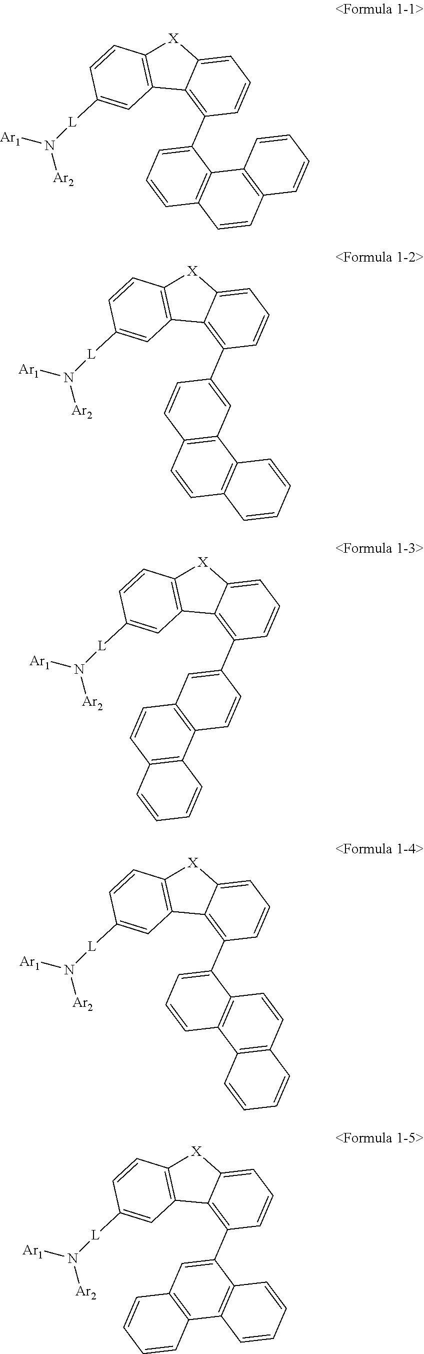
in Formula 1-1 to Formula 1-5, X, L, Ar₁ and Ar₂ are the same as defined in claim 1.

3. The compound of claim 1, wherein Formula 1 is represented by Formula 1-6 or Formula 1-7: 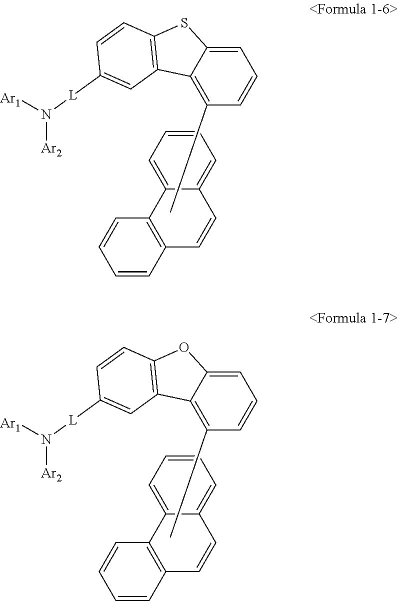
in Formula 1-6 and Formula 1-7, L, Ar₁ and Ar₂ are the same as defined in claim 1.

4. The compound of claim 1, wherein Formula 1 is represented by Formula 1-8: 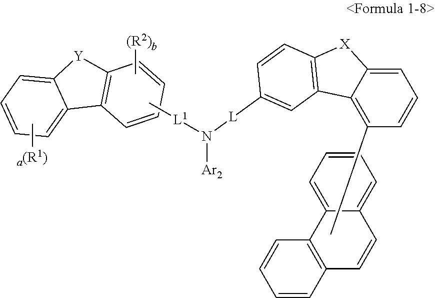
in Formula 1-8, X, L and Ar₂ are the same as defined in claim 1,
Y is O, S, C(R′)(R″) or N-(L₂-Ar₃),
R¹, R², R′ and R″ are each independently selected from the group consisting of hydrogen, deuterium, halogen, a silane group unsubstituted or substituted with a C₁-C₂₀ alkyl group or a C₆-C₂₀ aryl group, a phosphine oxide group unsubstituted or substituted with a C₁-C₂₀ alkyl group or a C₆-C₂₀ aryl group, siloxane group, a cyano group, a nitro group, a C₁-C₂₀ alkylthio group, a C₁-C₂₀ alkoxyl group, a C₆-C₂₀ aryloxy group, a C₆-C₂₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group containing at least one heteroatom of O, N, S, Si and P, and a C₃-C₃₀ aliphatic ring group, and adjacent groups may be bonded to each other to form a ring, and R′ and R″ may be bonded to each other to form a ring,
a is an integer of 0 to 4, b is an integer of 0 to 3, where a and b are each an integer of 2 or more, a plurality of R¹s and a plurality of R²s are each the same as or different from each other, and adjacent R¹s and adjacent R²s may be bonded to each other to form a ring,
L¹ and L₂ are each independently selected from the group consisting of a single bond, a C₆-C₃₀ arylene group, a fluorenylene group, a C₂-C₃₀ heterocyclic group containing at least one heteroatom of O, N, S, Si and P, and a C₃-C₃₀ aliphatic ring group, and
Ar₃ is selected from the group consisting of a C₆-C₃₀ aromatic ring group, a fluorenyl group, a C₂-C₃₀ heterocyclic group containing at least one heteroatom of O, N, S, Si and P, and a C₃-C₃₀ aliphatic ring.

5. The compound of claim 1, wherein both Ar₁ and Ar₂ are a C₆-C₆₀ aryl group.

6. The compound of claim 1, wherein the compound represented by Formula 1 is one of the following compounds:

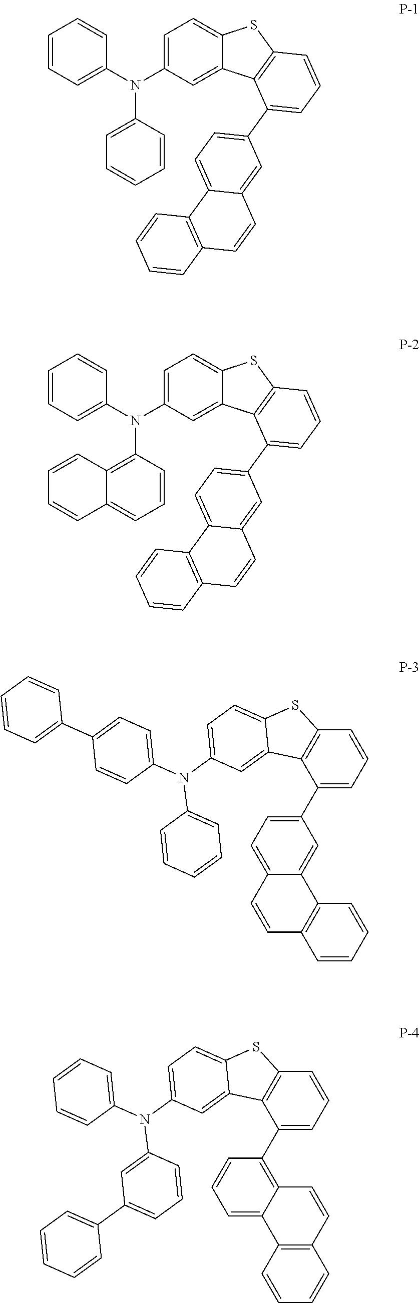 [SMILES: c1ccc(N(c2ccccc2)c2ccc3sc4cccc(-c5ccc6c(ccc7ccccc76)c5)c4c3c2)cc1 ; c1ccc(N(c2ccc3sc4cccc(-c5ccc6c(ccc7ccccc76)c5)c4c3c2)c2cccc3ccccc23)cc1 ; c1ccc(-c2ccc(N(c3ccccc3)c3ccc4sc5cccc(-c6ccc7ccc8ccccc8c7c6)c5c4c3)cc2)cc1 ; c1ccc(-c2cccc(N(c3ccccc3)c3ccc4sc5cccc(-c6cccc7c6ccc6ccccc67)c5c4c3)c2)cc1]

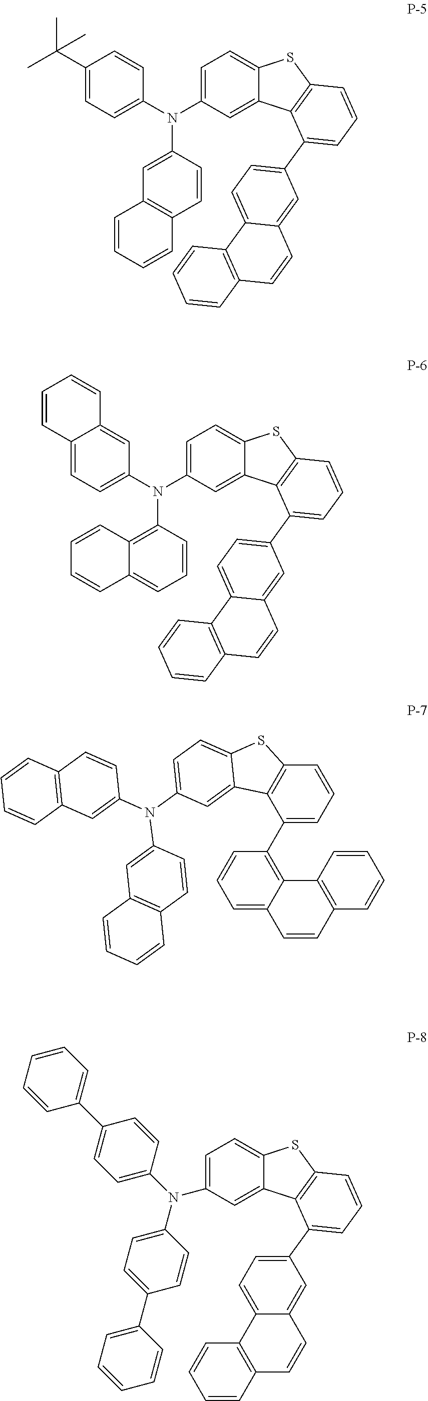 [SMILES: CC(C)(C)c1ccc(N(c2ccc3ccccc3c2)c2ccc3sc4cccc(-c5ccc6c(ccc7ccccc76)c5)c4c3c2)cc1 ; c1ccc2cc(N(c3ccc4sc5cccc(-c6ccc7c(ccc8ccccc87)c6)c5c4c3)c3cccc4ccccc34)ccc2c1 ; c1ccc2cc(N(c3ccc4ccccc4c3)c3ccc4sc5cccc(-c6cccc7ccc8ccccc8c67)c5c4c3)ccc2c1 ; c1ccc(-c2ccc(N(c3ccc(-c4ccccc4)cc3)c3ccc4sc5cccc(-c6ccc7c(ccc8ccccc87)c6)c5c4c3)cc2)cc1]

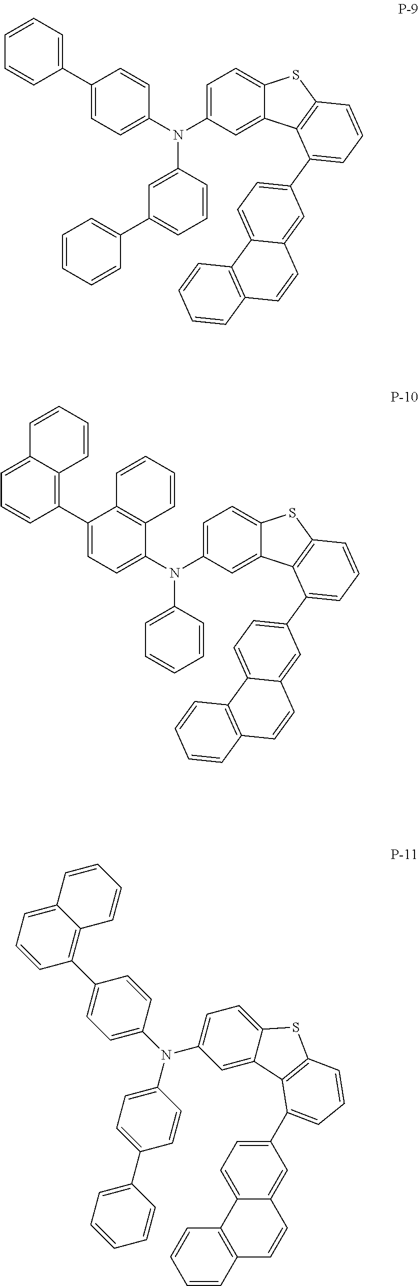 [SMILES: c1ccc(-c2ccc(N(c3cccc(-c4ccccc4)c3)c3ccc4sc5cccc(-c6ccc7c(ccc8ccccc87)c6)c5c4c3)cc2)cc1 ; c1ccc(N(c2ccc3sc4cccc(-c5ccc6c(ccc7ccccc76)c5)c4c3c2)c2ccc(-c3cccc4ccccc34)c3ccccc23)cc1 ; c1ccc(-c2ccc(N(c3ccc(-c4cccc5ccccc45)cc3)c3ccc4sc5cccc(-c6ccc7c(ccc8ccccc87)c6)c5c4c3)cc2)cc1]

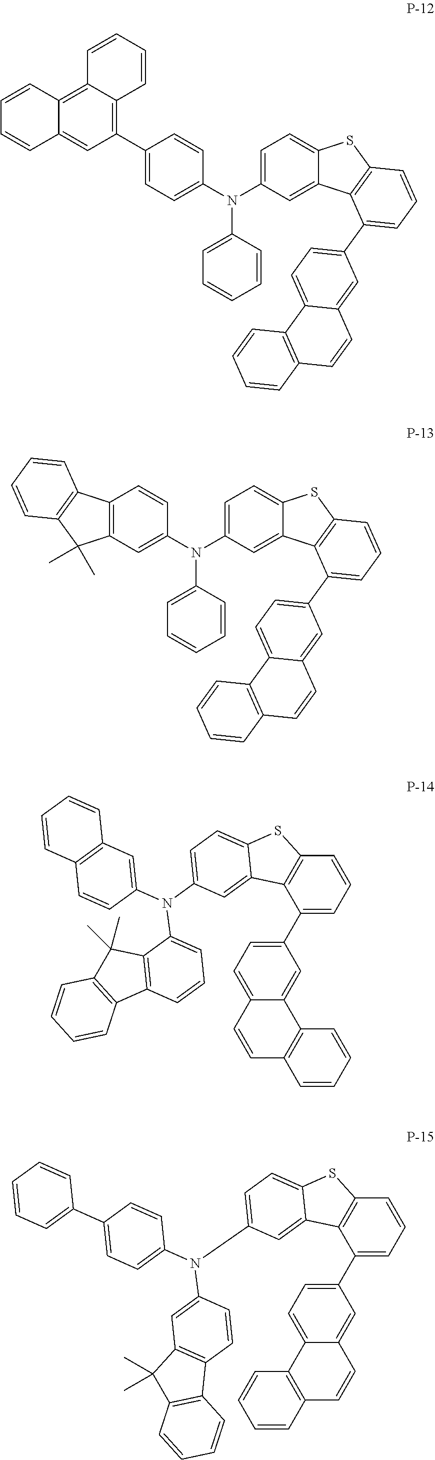 [SMILES: c1ccc(N(c2ccc(-c3cc4ccccc4c4ccccc34)cc2)c2ccc3sc4cccc(-c5ccc6c(ccc7ccccc76)c5)c4c3c2)cc1 ; CC1(C)c2ccccc2-c2ccc(N(c3ccccc3)c3ccc4sc5cccc(-c6ccc7c(ccc8ccccc87)c6)c5c4c3)cc21 ; CC1(C)c2ccccc2-c2cccc(N(c3ccc4ccccc4c3)c3ccc4sc5cccc(-c6ccc7ccc8ccccc8c7c6)c5c4c3)c21 ; CC1(C)c2ccccc2-c2ccc(N(c3ccc(-c4ccccc4)cc3)c3ccc4sc5cccc(-c6ccc7c(ccc8ccccc87)c6)c5c4c3)cc21]

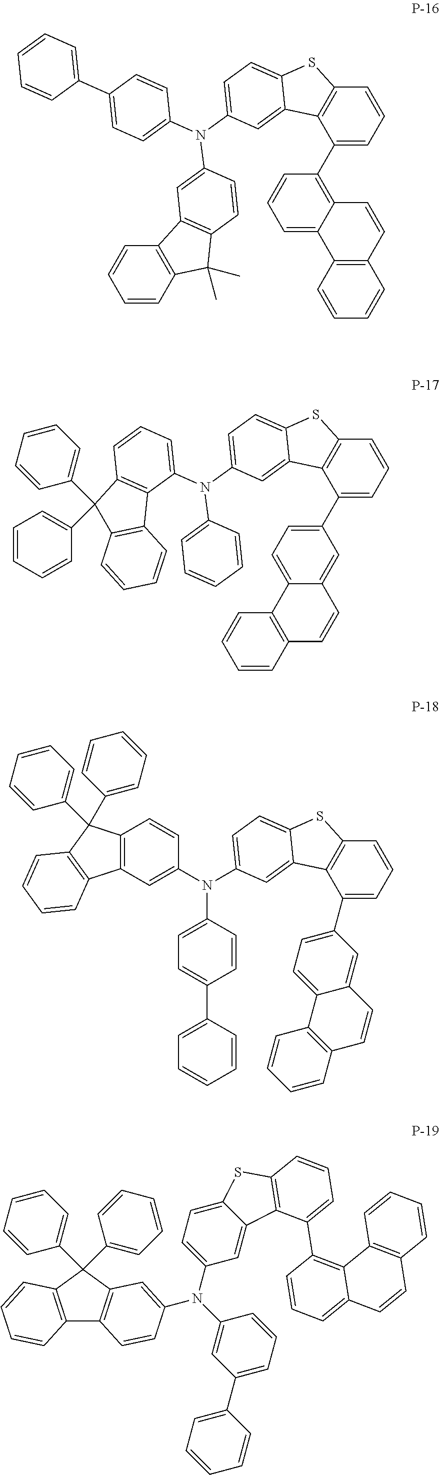 [SMILES: CC1(C)c2ccccc2-c2cc(N(c3ccc(-c4ccccc4)cc3)c3ccc4sc5cccc(-c6cccc7c6ccc6ccccc67)c5c4c3)ccc21 ; c1ccc(N(c2ccc3sc4cccc(-c5ccc6c(ccc7ccccc76)c5)c4c3c2)c2cccc3c2-c2ccccc2C3(c2ccccc2)c2ccccc2)cc1 ; c1ccc(-c2ccc(N(c3ccc4c(c3)-c3ccccc3C4(c3ccccc3)c3ccccc3)c3ccc4sc5cccc(-c6ccc7c(ccc8ccccc87)c6)c5c4c3)cc2)cc1 ; c1ccc(-c2cccc(N(c3ccc4c(c3)C(c3ccccc3)(c3ccccc3)c3ccccc3-4)c3ccc4sc5cccc(-c6cccc7ccc8ccccc8c67)c5c4c3)c2)cc1]

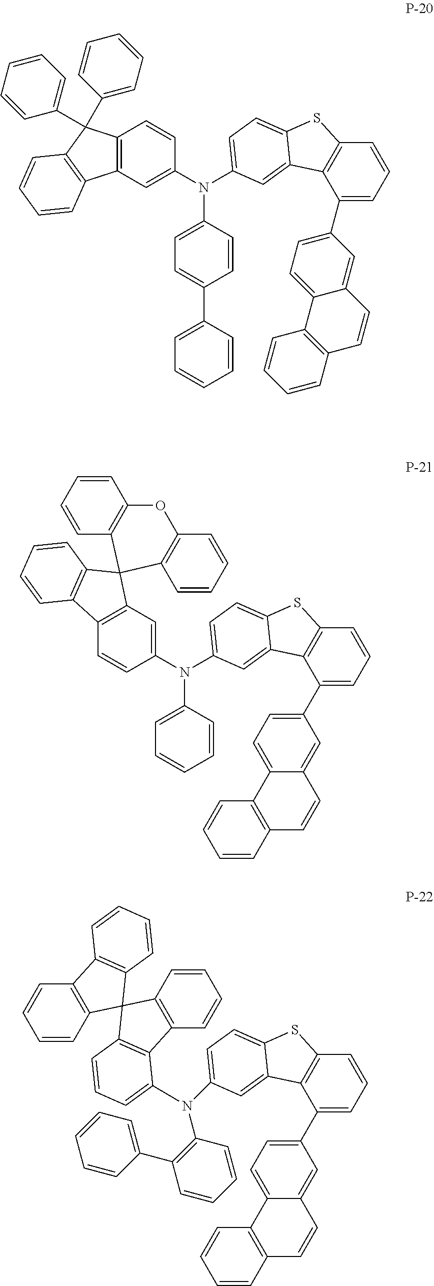 [SMILES: c1ccc(-c2ccc(N(c3ccc4c(c3)-c3ccccc3C4(c3ccccc3)c3ccccc3)c3ccc4sc5cccc(-c6ccc7c(ccc8ccccc87)c6)c5c4c3)cc2)cc1 ; c1ccc(N(c2ccc3c(c2)C2(c4ccccc4Oc4ccccc42)c2ccccc2-3)c2ccc3sc4cccc(-c5ccc6c(ccc7ccccc76)c5)c4c3c2)cc1 ; c1ccc(-c2ccccc2N(c2ccc3sc4cccc(-c5ccc6c(ccc7ccccc76)c5)c4c3c2)c2cccc3c2-c2ccccc2C32c3ccccc3-c3ccccc32)cc1]

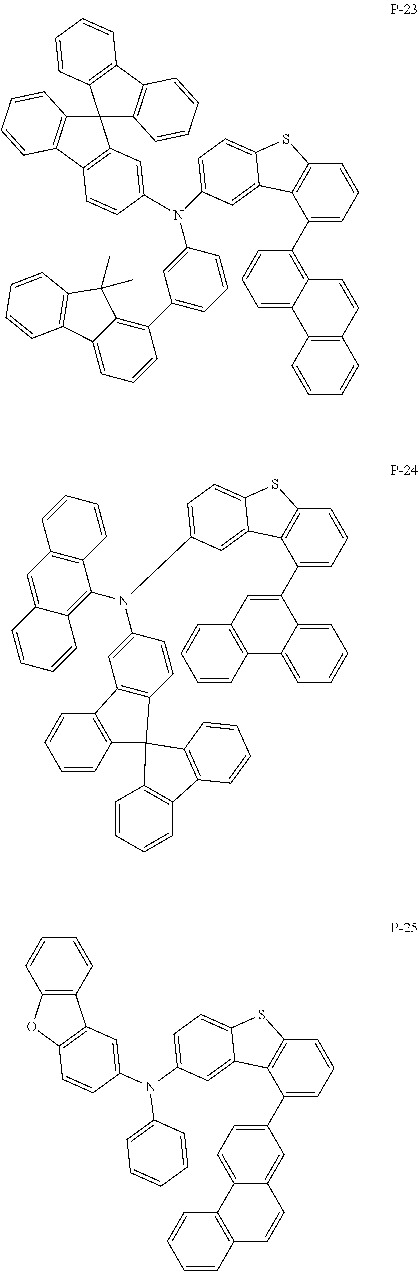 [SMILES: CC1(C)c2ccccc2-c2cccc(-c3cccc(N(c4ccc5c(c4)C4(c6ccccc6-c6ccccc64)c4ccccc4-5)c4ccc5sc6cccc(-c7cccc8c7ccc7ccccc78)c6c5c4)c3)c21 ; c1ccc2c(c1)-c1ccccc1C21c2ccccc2-c2cc(N(c3ccc4sc5cccc(-c6cc7ccccc7c7ccccc67)c5c4c3)c3c4ccccc4cc4ccccc34)ccc21 ; c1ccc(N(c2ccc3oc4ccccc4c3c2)c2ccc3sc4cccc(-c5ccc6c(ccc7ccccc76)c5)c4c3c2)cc1]

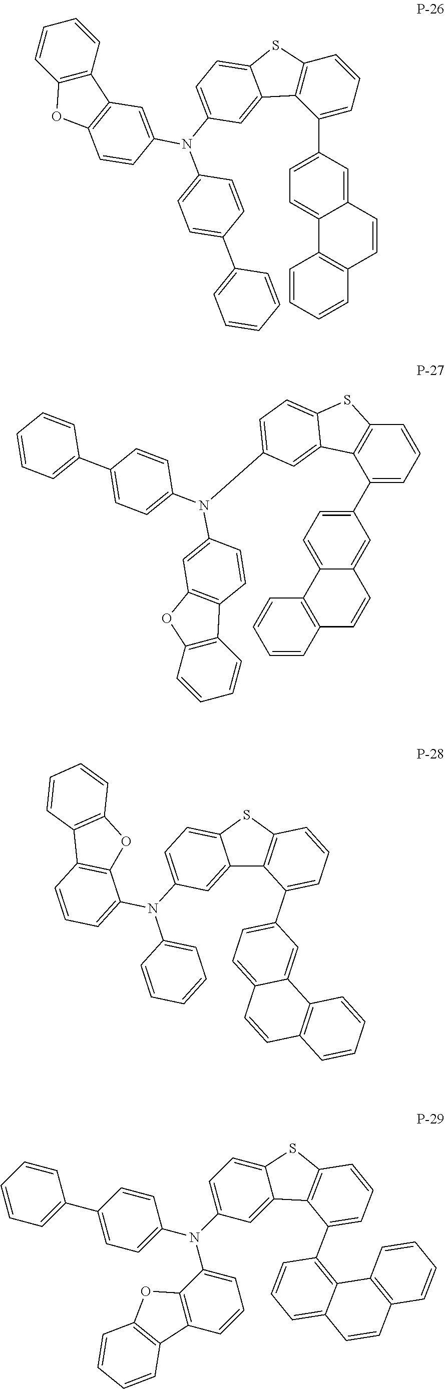 [SMILES: c1ccc(-c2ccc(N(c3ccc4oc5ccccc5c4c3)c3ccc4sc5cccc(-c6ccc7c(ccc8ccccc87)c6)c5c4c3)cc2)cc1 ; c1ccc(-c2ccc(N(c3ccc4c(c3)oc3ccccc34)c3ccc4sc5cccc(-c6ccc7c(ccc8ccccc87)c6)c5c4c3)cc2)cc1 ; c1ccc(N(c2ccc3sc4cccc(-c5ccc6ccc7ccccc7c6c5)c4c3c2)c2cccc3c2oc2ccccc23)cc1 ; c1ccc(-c2ccc(N(c3ccc4sc5cccc(-c6cccc7ccc8ccccc8c67)c5c4c3)c3cccc4c3oc3ccccc34)cc2)cc1]

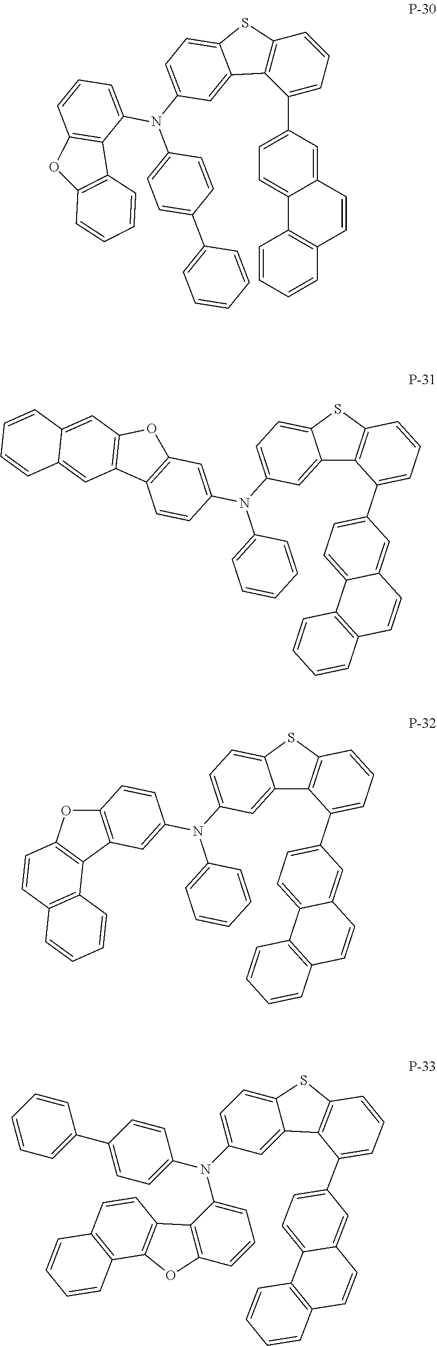 [SMILES: c1ccc(-c2ccc(N(c3ccc4sc5cccc(-c6ccc7c(ccc8ccccc87)c6)c5c4c3)c3cccc4oc5ccccc5c34)cc2)cc1 ; c1ccc(N(c2ccc3c(c2)oc2cc4ccccc4cc23)c2ccc3sc4cccc(-c5ccc6c(ccc7ccccc76)c5)c4c3c2)cc1 ; c1ccc(N(c2ccc3sc4cccc(-c5ccc6c(ccc7ccccc76)c5)c4c3c2)c2ccc3oc4ccc5ccccc5c4c3c2)cc1 ; c1ccc(-c2ccc(N(c3ccc4sc5cccc(-c6ccc7c(ccc8ccccc87)c6)c5c4c3)c3cccc4oc5c6ccccc6ccc5c34)cc2)cc1]

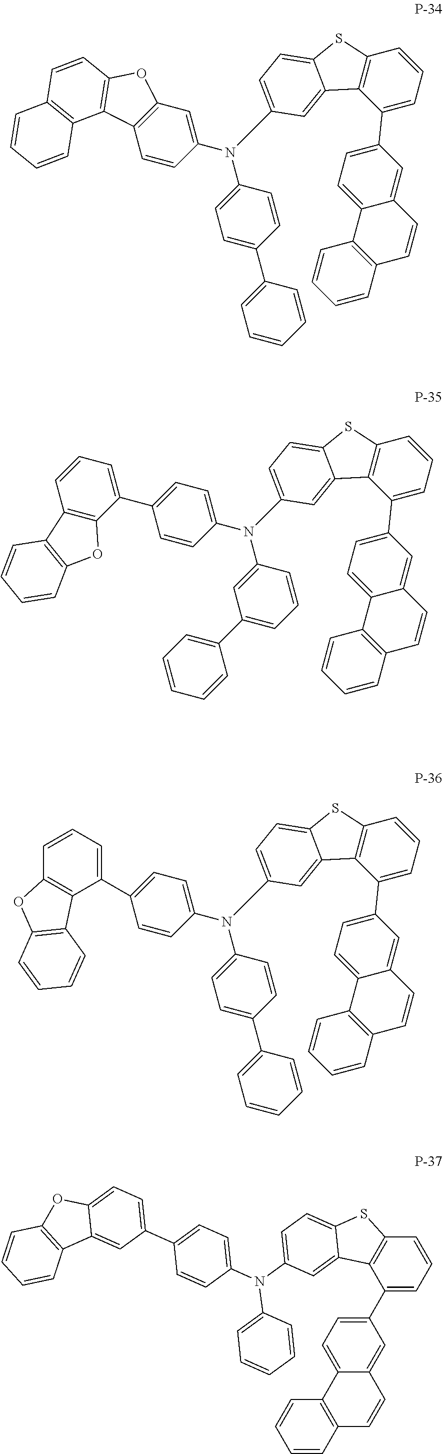 [SMILES: c1ccc(-c2ccc(N(c3ccc4c(c3)oc3ccc5ccccc5c34)c3ccc4sc5cccc(-c6ccc7c(ccc8ccccc87)c6)c5c4c3)cc2)cc1 ; c1ccc(-c2cccc(N(c3ccc(-c4cccc5c4oc4ccccc45)cc3)c3ccc4sc5cccc(-c6ccc7c(ccc8ccccc87)c6)c5c4c3)c2)cc1 ; c1ccc(-c2ccc(N(c3ccc(-c4cccc5oc6ccccc6c45)cc3)c3ccc4sc5cccc(-c6ccc7c(ccc8ccccc87)c6)c5c4c3)cc2)cc1 ; c1ccc(N(c2ccc(-c3ccc4oc5ccccc5c4c3)cc2)c2ccc3sc4cccc(-c5ccc6c(ccc7ccccc76)c5)c4c3c2)cc1]

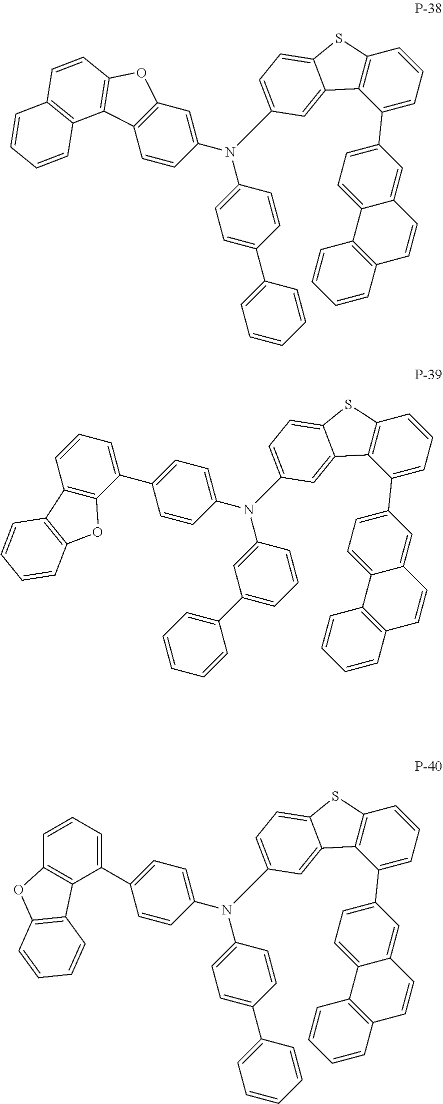 [SMILES: c1ccc(-c2ccc(N(c3ccc4c(c3)oc3ccc5ccccc5c34)c3ccc4sc5cccc(-c6ccc7c(ccc8ccccc87)c6)c5c4c3)cc2)cc1 ; c1ccc(-c2cccc(N(c3ccc(-c4cccc5c4oc4ccccc45)cc3)c3ccc4sc5cccc(-c6ccc7c(ccc8ccccc87)c6)c5c4c3)c2)cc1 ; c1ccc(-c2ccc(N(c3ccc(-c4cccc5oc6ccccc6c45)cc3)c3ccc4sc5cccc(-c6ccc7c(ccc8ccccc87)c6)c5c4c3)cc2)cc1]

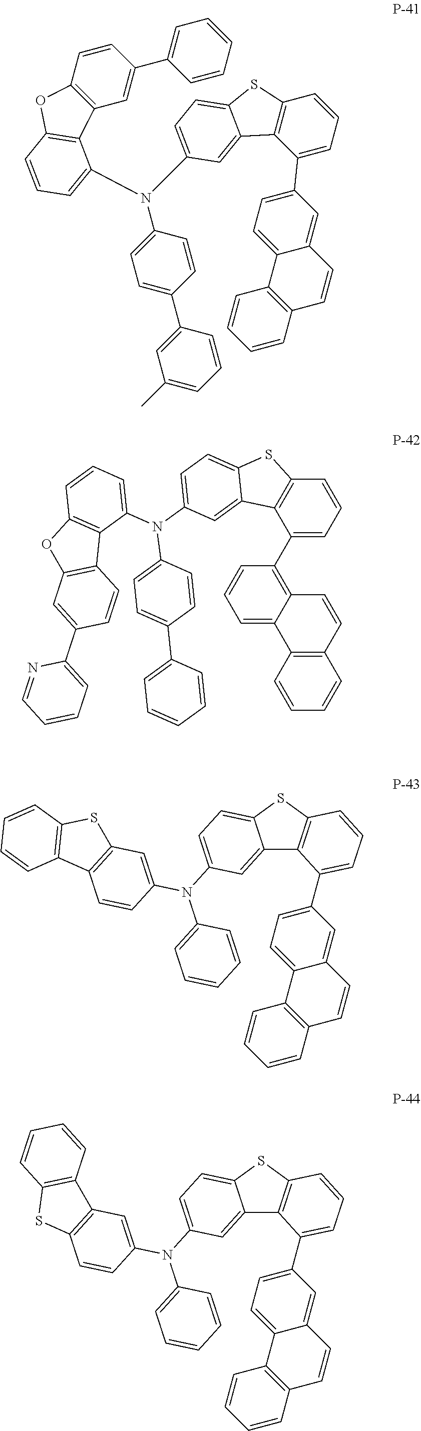 [SMILES: Cc1cccc(-c2ccc(N(c3ccc4sc5cccc(-c6ccc7c(ccc8ccccc87)c6)c5c4c3)c3cccc4oc5ccc(-c6ccccc6)cc5c34)cc2)c1 ; c1ccc(-c2ccc(N(c3ccc4sc5cccc(-c6cccc7c6ccc6ccccc67)c5c4c3)c3cccc4oc5cc(-c6ccccn6)ccc5c34)cc2)cc1 ; c1ccc(N(c2ccc3c(c2)sc2ccccc23)c2ccc3sc4cccc(-c5ccc6c(ccc7ccccc76)c5)c4c3c2)cc1 ; c1ccc(N(c2ccc3sc4ccccc4c3c2)c2ccc3sc4cccc(-c5ccc6c(ccc7ccccc76)c5)c4c3c2)cc1]

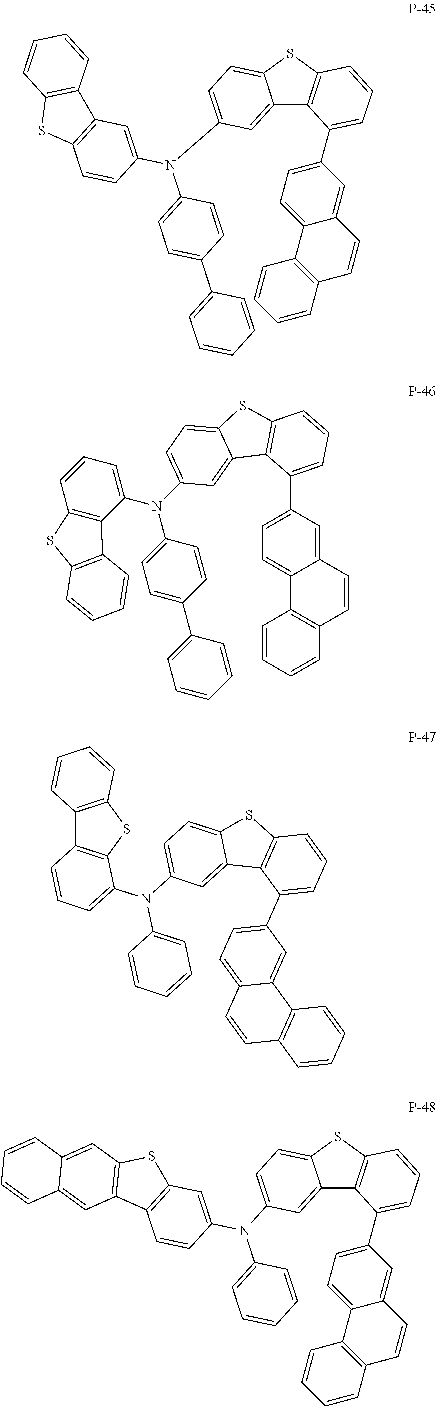 [SMILES: c1ccc(-c2ccc(N(c3ccc4sc5ccccc5c4c3)c3ccc4sc5cccc(-c6ccc7c(ccc8ccccc87)c6)c5c4c3)cc2)cc1 ; c1ccc(-c2ccc(N(c3ccc4sc5cccc(-c6ccc7c(ccc8ccccc87)c6)c5c4c3)c3cccc4sc5ccccc5c34)cc2)cc1 ; c1ccc(N(c2ccc3sc4cccc(-c5ccc6ccc7ccccc7c6c5)c4c3c2)c2cccc3c2sc2ccccc23)cc1 ; c1ccc(N(c2ccc3c(c2)sc2cc4ccccc4cc23)c2ccc3sc4cccc(-c5ccc6c(ccc7ccccc76)c5)c4c3c2)cc1]

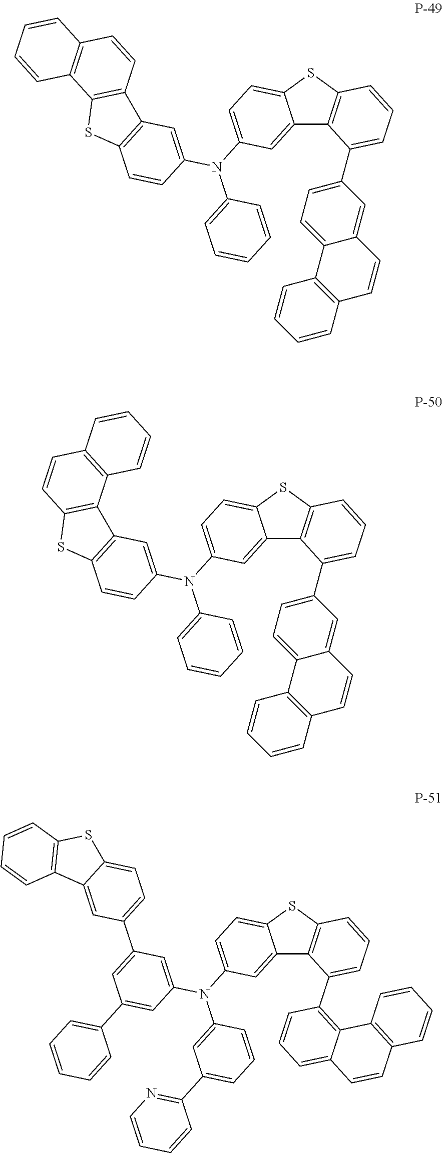 [SMILES: c1ccc(N(c2ccc3sc4c5ccccc5ccc4c3c2)c2ccc3sc4cccc(-c5ccc6c(ccc7ccccc76)c5)c4c3c2)cc1 ; c1ccc(N(c2ccc3sc4cccc(-c5ccc6c(ccc7ccccc76)c5)c4c3c2)c2ccc3sc4ccc5ccccc5c4c3c2)cc1 ; c1ccc(-c2cc(-c3ccc4sc5ccccc5c4c3)cc(N(c3cccc(-c4ccccn4)c3)c3ccc4sc5cccc(-c6cccc7ccc8ccccc8c67)c5c4c3)c2)cc1]

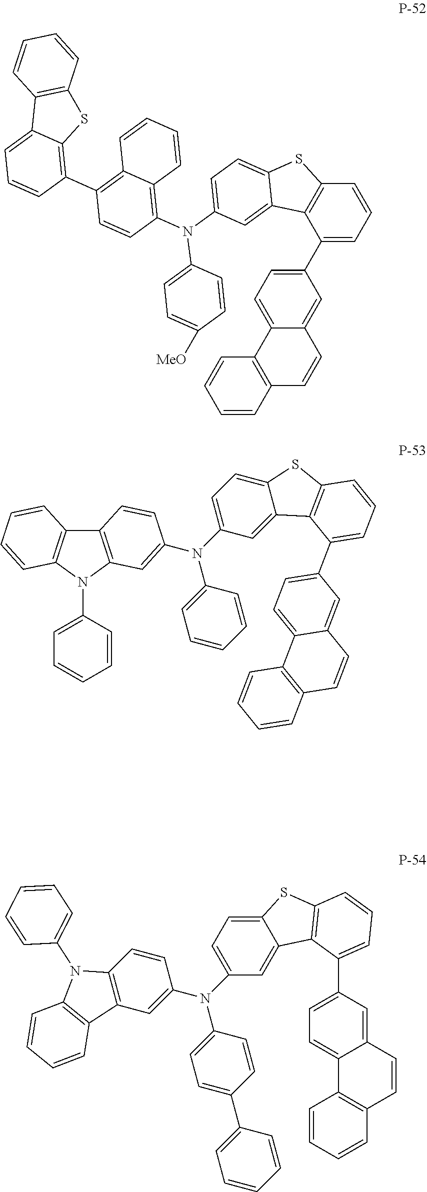 [SMILES: COc1ccc(N(c2ccc3sc4cccc(-c5ccc6c(ccc7ccccc76)c5)c4c3c2)c2ccc(-c3cccc4c3sc3ccccc34)c3ccccc23)cc1 ; c1ccc(N(c2ccc3sc4cccc(-c5ccc6c(ccc7ccccc76)c5)c4c3c2)c2ccc3c4ccccc4n(-c4ccccc4)c3c2)cc1 ; c1ccc(-c2ccc(N(c3ccc4sc5cccc(-c6ccc7c(ccc8ccccc87)c6)c5c4c3)c3ccc4c(c3)c3ccccc3n4-c3ccccc3)cc2)cc1]

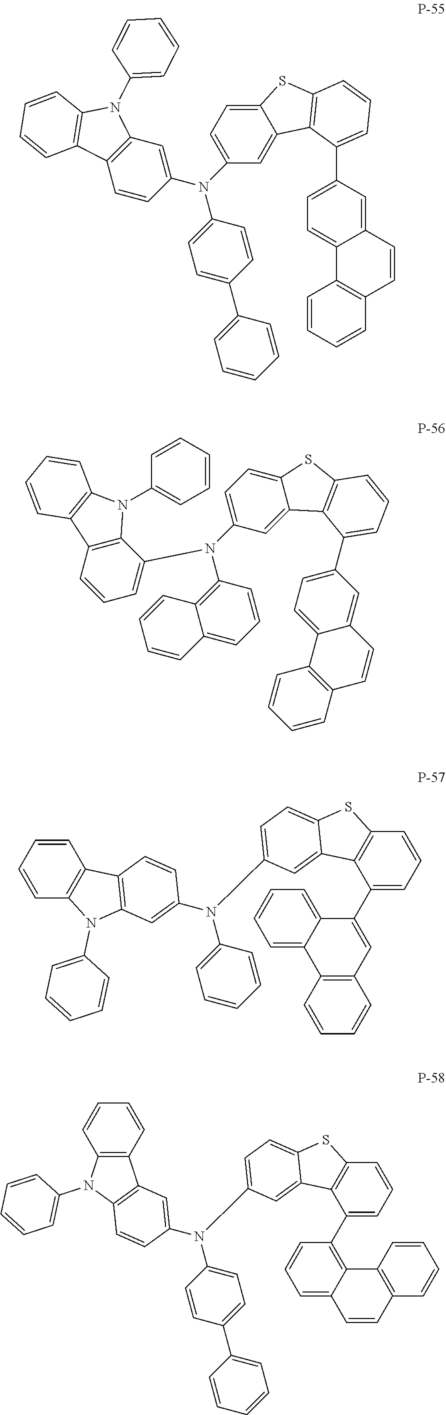 [SMILES: c1ccc(-c2ccc(N(c3ccc4sc5cccc(-c6ccc7c(ccc8ccccc87)c6)c5c4c3)c3ccc4c5ccccc5n(-c5ccccc5)c4c3)cc2)cc1 ; c1ccc(-n2c3ccccc3c3cccc(N(c4ccc5sc6cccc(-c7ccc8c(ccc9ccccc98)c7)c6c5c4)c4cccc5ccccc45)c32)cc1 ; c1ccc(N(c2ccc3sc4cccc(-c5cc6ccccc6c6ccccc56)c4c3c2)c2ccc3c4ccccc4n(-c4ccccc4)c3c2)cc1 ; c1ccc(-c2ccc(N(c3ccc4sc5cccc(-c6cccc7ccc8ccccc8c67)c5c4c3)c3ccc4c(c3)c3ccccc3n4-c3ccccc3)cc2)cc1]

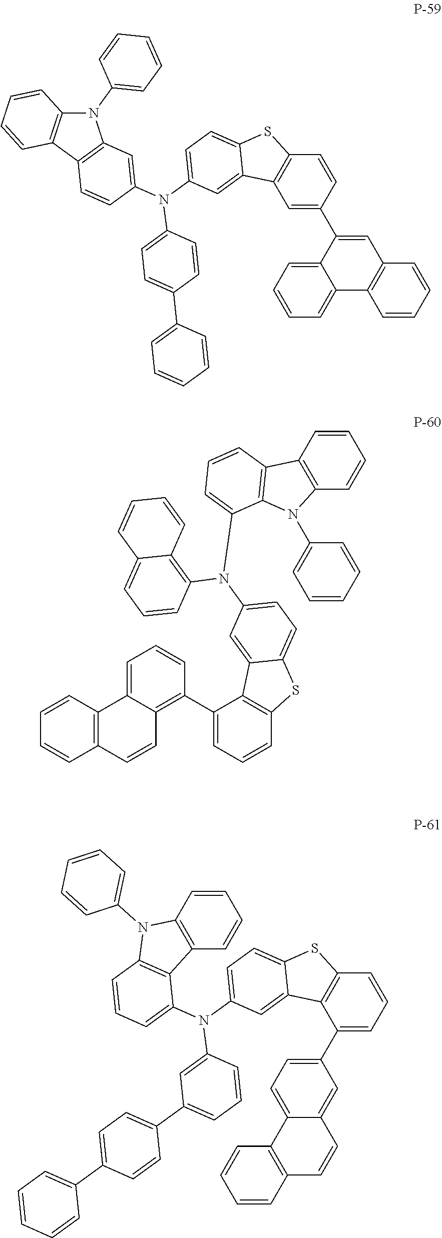 [SMILES: c1ccc(-c2ccc(N(c3ccc4sc5ccc(-c6cc7ccccc7c7ccccc67)cc5c4c3)c3ccc4c5ccccc5n(-c5ccccc5)c4c3)cc2)cc1 ; c1ccc(-n2c3ccccc3c3cccc(N(c4ccc5sc6cccc(-c7cccc8c7ccc7ccccc78)c6c5c4)c4cccc5ccccc45)c32)cc1 ; c1ccc(-c2ccc(-c3cccc(N(c4ccc5sc6cccc(-c7ccc8c(ccc9ccccc98)c7)c6c5c4)c4cccc5c4c4ccccc4n5-c4ccccc4)c3)cc2)cc1]

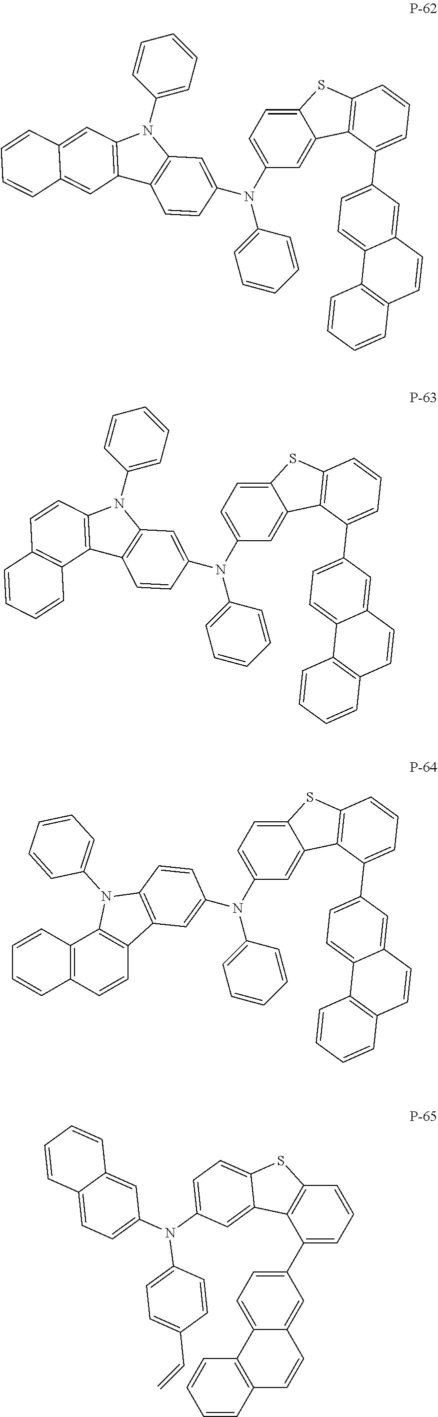 [SMILES: c1ccc(N(c2ccc3sc4cccc(-c5ccc6c(ccc7ccccc76)c5)c4c3c2)c2ccc3c4cc5ccccc5cc4n(-c4ccccc4)c3c2)cc1 ; c1ccc(N(c2ccc3sc4cccc(-c5ccc6c(ccc7ccccc76)c5)c4c3c2)c2ccc3c4c5ccccc5ccc4n(-c4ccccc4)c3c2)cc1 ; c1ccc(N(c2ccc3sc4cccc(-c5ccc6c(ccc7ccccc76)c5)c4c3c2)c2ccc3c(c2)c2ccc4ccccc4c2n3-c2ccccc2)cc1 ; C=Cc1ccc(N(c2ccc3ccccc3c2)c2ccc3sc4cccc(-c5ccc6c(ccc7ccccc76)c5)c4c3c2)cc1]

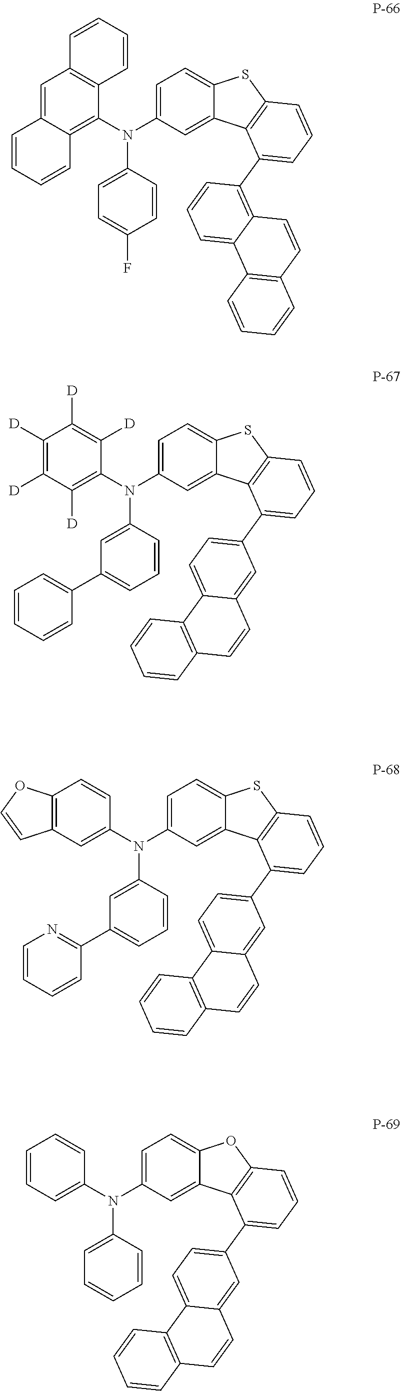 [SMILES: Fc1ccc(N(c2ccc3sc4cccc(-c5cccc6c5ccc5ccccc56)c4c3c2)c2c3ccccc3cc3ccccc23)cc1 ; [2H]c1c([2H])c([2H])c(N(c2cccc(-c3ccccc3)c2)c2ccc3sc4cccc(-c5ccc6c(ccc7ccccc76)c5)c4c3c2)c([2H])c1[2H] ; c1ccc(-c2cccc(N(c3ccc4occc4c3)c3ccc4sc5cccc(-c6ccc7c(ccc8ccccc87)c6)c5c4c3)c2)nc1 ; c1ccc(N(c2ccccc2)c2ccc3oc4cccc(-c5ccc6c(ccc7ccccc76)c5)c4c3c2)cc1]

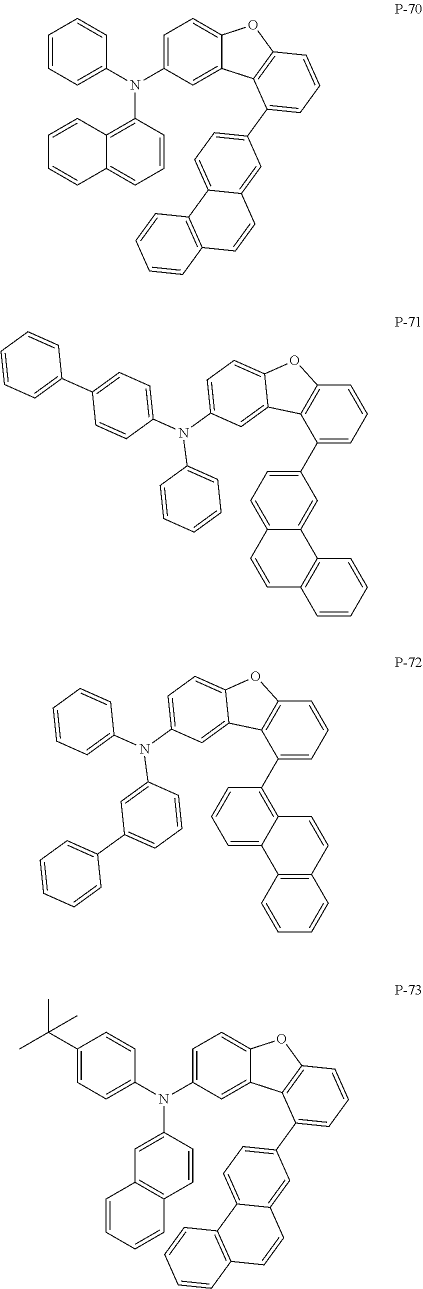 [SMILES: c1ccc(N(c2ccc3oc4cccc(-c5ccc6c(ccc7ccccc76)c5)c4c3c2)c2cccc3ccccc23)cc1 ; c1ccc(-c2ccc(N(c3ccccc3)c3ccc4oc5cccc(-c6ccc7ccc8ccccc8c7c6)c5c4c3)cc2)cc1 ; c1ccc(-c2cccc(N(c3ccccc3)c3ccc4oc5cccc(-c6cccc7c6ccc6ccccc67)c5c4c3)c2)cc1 ; CC(C)(C)c1ccc(N(c2ccc3ccccc3c2)c2ccc3oc4cccc(-c5ccc6c(ccc7ccccc76)c5)c4c3c2)cc1]

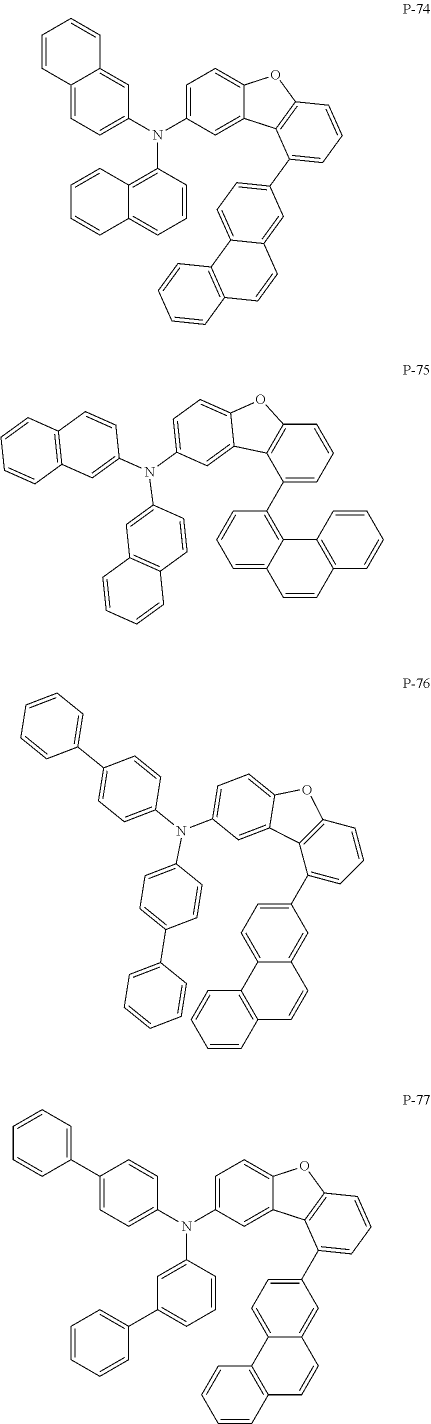 [SMILES: c1ccc2cc(N(c3ccc4oc5cccc(-c6ccc7c(ccc8ccccc87)c6)c5c4c3)c3cccc4ccccc34)ccc2c1 ; c1ccc2cc(N(c3ccc4ccccc4c3)c3ccc4oc5cccc(-c6cccc7ccc8ccccc8c67)c5c4c3)ccc2c1 ; c1ccc(-c2ccc(N(c3ccc(-c4ccccc4)cc3)c3ccc4oc5cccc(-c6ccc7c(ccc8ccccc87)c6)c5c4c3)cc2)cc1 ; c1ccc(-c2ccc(N(c3cccc(-c4ccccc4)c3)c3ccc4oc5cccc(-c6ccc7c(ccc8ccccc87)c6)c5c4c3)cc2)cc1]

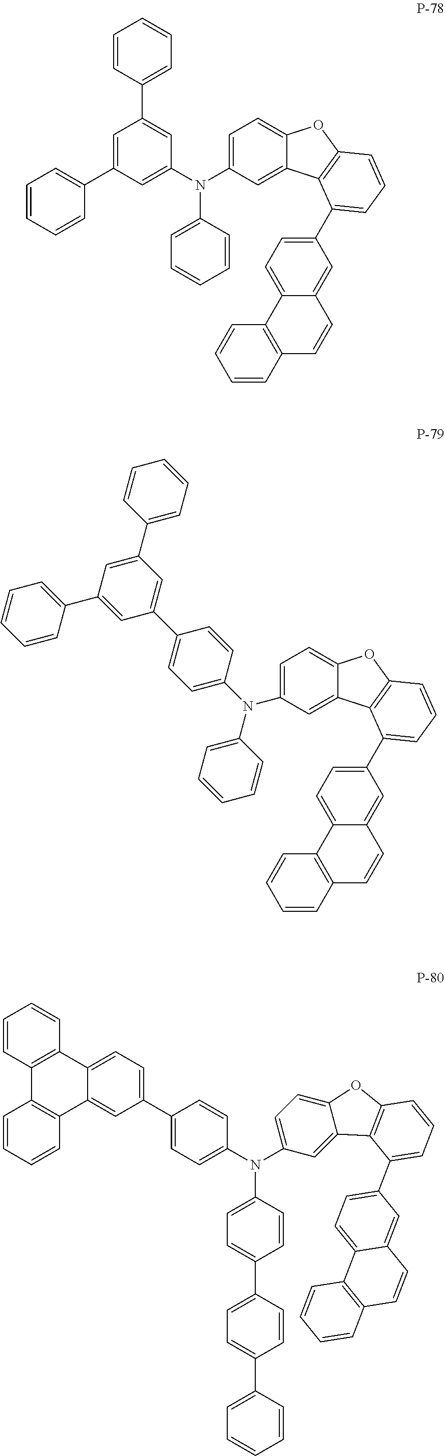 [SMILES: c1ccc(-c2cc(-c3ccccc3)cc(N(c3ccccc3)c3ccc4oc5cccc(-c6ccc7c(ccc8ccccc87)c6)c5c4c3)c2)cc1 ; c1ccc(-c2cc(-c3ccccc3)cc(-c3ccc(N(c4ccccc4)c4ccc5oc6cccc(-c7ccc8c(ccc9ccccc98)c7)c6c5c4)cc3)c2)cc1 ; c1ccc(-c2ccc(-c3ccc(N(c4ccc(-c5ccc6c7ccccc7c7ccccc7c6c5)cc4)c4ccc5oc6cccc(-c7ccc8c(ccc9ccccc98)c7)c6c5c4)cc3)cc2)cc1]

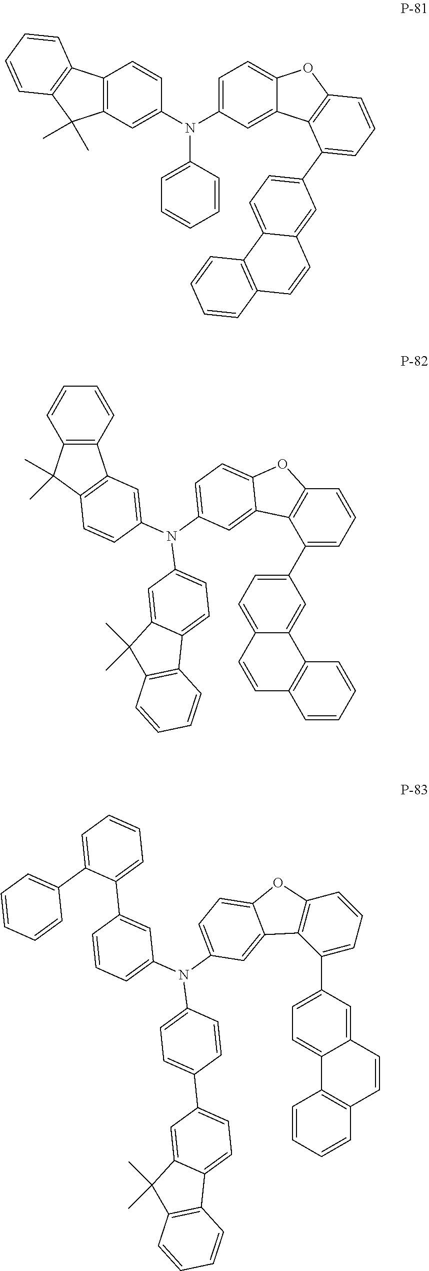 [SMILES: CC1(C)c2ccccc2-c2ccc(N(c3ccccc3)c3ccc4oc5cccc(-c6ccc7c(ccc8ccccc87)c6)c5c4c3)cc21 ; CC1(C)c2ccccc2-c2cc(N(c3ccc4c(c3)C(C)(C)c3ccccc3-4)c3ccc4oc5cccc(-c6ccc7ccc8ccccc8c7c6)c5c4c3)ccc21 ; CC1(C)c2ccccc2-c2ccc(-c3ccc(N(c4cccc(-c5ccccc5-c5ccccc5)c4)c4ccc5oc6cccc(-c7ccc8c(ccc9ccccc98)c7)c6c5c4)cc3)cc21]

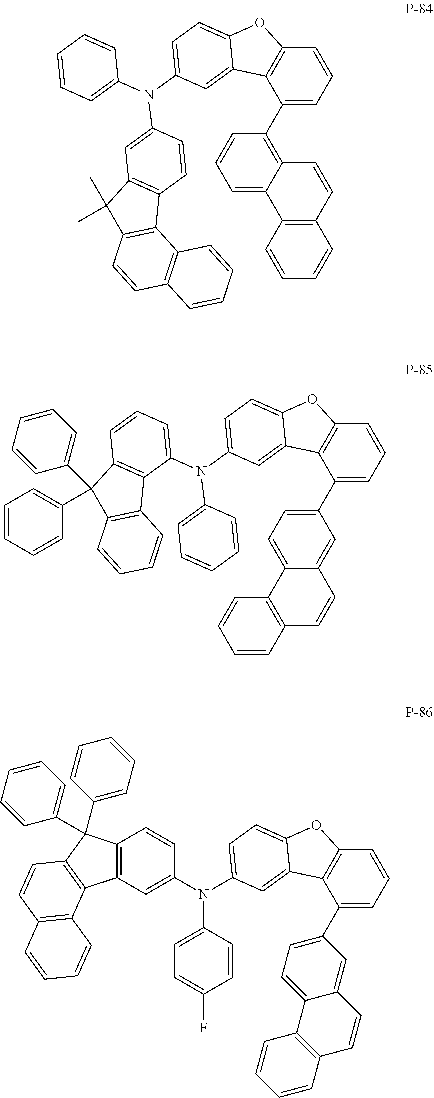 [SMILES: CC1(C)c2cc(N(c3ccccc3)c3ccc4oc5cccc(-c6cccc7c6ccc6ccccc67)c5c4c3)ccc2-c2c1ccc1ccccc21 ; c1ccc(N(c2ccc3oc4cccc(-c5ccc6c(ccc7ccccc76)c5)c4c3c2)c2cccc3c2-c2ccccc2C3(c2ccccc2)c2ccccc2)cc1 ; Fc1ccc(N(c2ccc3c(c2)-c2c(ccc4ccccc24)C3(c2ccccc2)c2ccccc2)c2ccc3oc4cccc(-c5ccc6c(ccc7ccccc76)c5)c4c3c2)cc1]

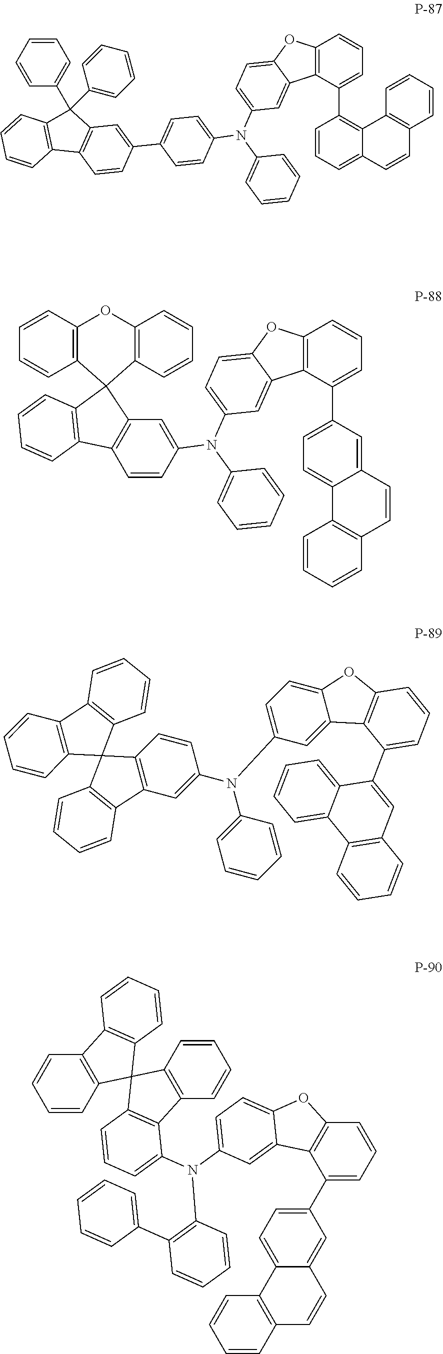 [SMILES: c1ccc(N(c2ccc(-c3ccc4c(c3)C(c3ccccc3)(c3ccccc3)c3ccccc3-4)cc2)c2ccc3oc4cccc(-c5cccc6ccc7ccccc7c56)c4c3c2)cc1 ; c1ccc(N(c2ccc3c(c2)C2(c4ccccc4Oc4ccccc42)c2ccccc2-3)c2ccc3oc4cccc(-c5ccc6c(ccc7ccccc76)c5)c4c3c2)cc1 ; c1ccc(N(c2ccc3c(c2)-c2ccccc2C32c3ccccc3-c3ccccc32)c2ccc3oc4cccc(-c5cc6ccccc6c6ccccc56)c4c3c2)cc1 ; c1ccc(-c2ccccc2N(c2ccc3oc4cccc(-c5ccc6c(ccc7ccccc76)c5)c4c3c2)c2cccc3c2-c2ccccc2C32c3ccccc3-c3ccccc32)cc1]

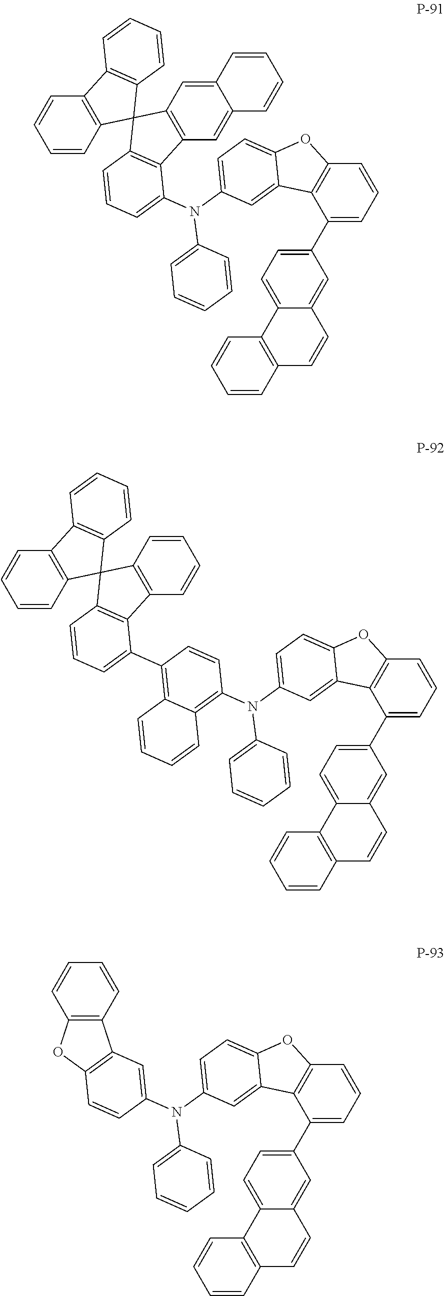 [SMILES: c1ccc(N(c2ccc3oc4cccc(-c5ccc6c(ccc7ccccc76)c5)c4c3c2)c2cccc3c2-c2cc4ccccc4cc2C32c3ccccc3-c3ccccc32)cc1 ; c1ccc(N(c2ccc3oc4cccc(-c5ccc6c(ccc7ccccc76)c5)c4c3c2)c2ccc(-c3cccc4c3-c3ccccc3C43c4ccccc4-c4ccccc43)c3ccccc23)cc1 ; c1ccc(N(c2ccc3oc4ccccc4c3c2)c2ccc3oc4cccc(-c5ccc6c(ccc7ccccc76)c5)c4c3c2)cc1]

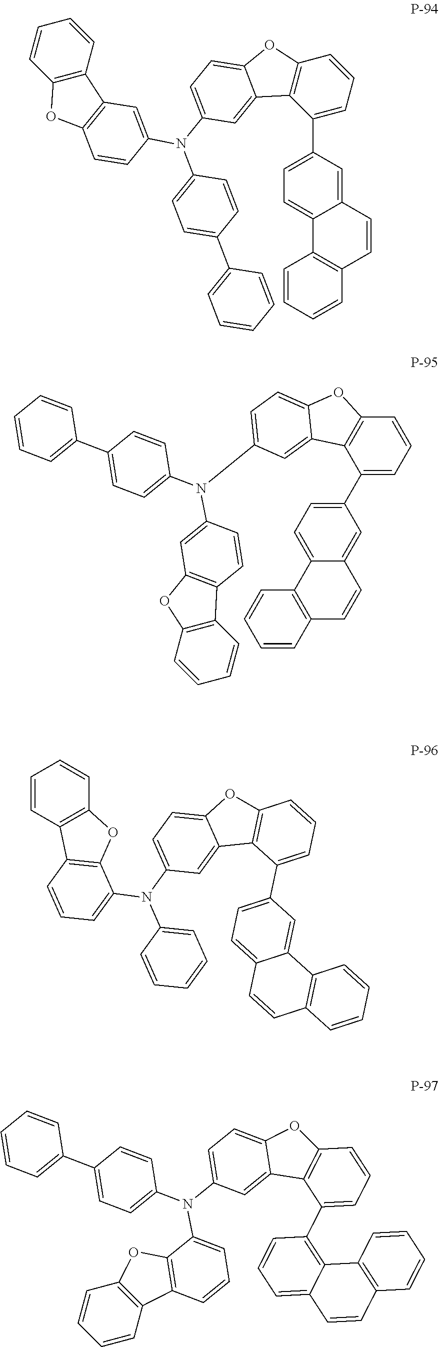 [SMILES: c1ccc(-c2ccc(N(c3ccc4oc5ccccc5c4c3)c3ccc4oc5cccc(-c6ccc7c(ccc8ccccc87)c6)c5c4c3)cc2)cc1 ; c1ccc(-c2ccc(N(c3ccc4c(c3)oc3ccccc34)c3ccc4oc5cccc(-c6ccc7c(ccc8ccccc87)c6)c5c4c3)cc2)cc1 ; c1ccc(N(c2ccc3oc4cccc(-c5ccc6ccc7ccccc7c6c5)c4c3c2)c2cccc3c2oc2ccccc23)cc1 ; c1ccc(-c2ccc(N(c3ccc4oc5cccc(-c6cccc7ccc8ccccc8c67)c5c4c3)c3cccc4c3oc3ccccc34)cc2)cc1]

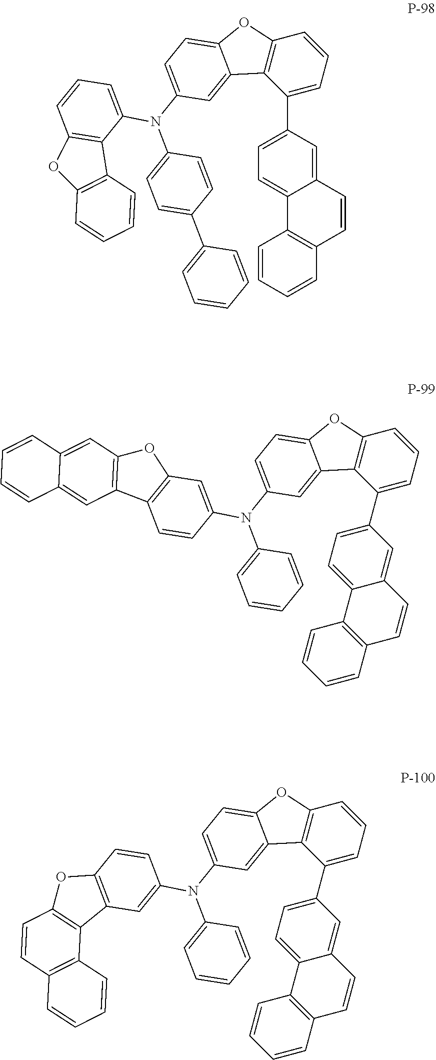 [SMILES: c1ccc(-c2ccc(N(c3ccc4oc5cccc(-c6ccc7c(ccc8ccccc87)c6)c5c4c3)c3cccc4oc5ccccc5c34)cc2)cc1 ; c1ccc(N(c2ccc3c(c2)oc2cc4ccccc4cc23)c2ccc3oc4cccc(-c5ccc6c(ccc7ccccc76)c5)c4c3c2)cc1 ; c1ccc(N(c2ccc3oc4cccc(-c5ccc6c(ccc7ccccc76)c5)c4c3c2)c2ccc3oc4ccc5ccccc5c4c3c2)cc1]

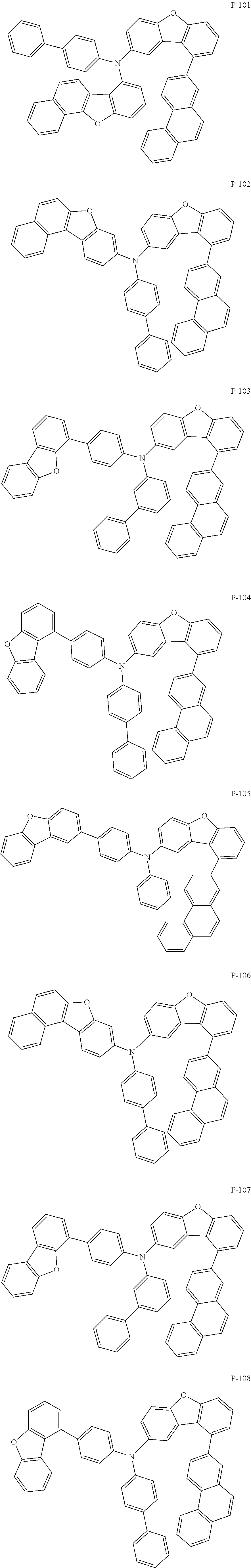

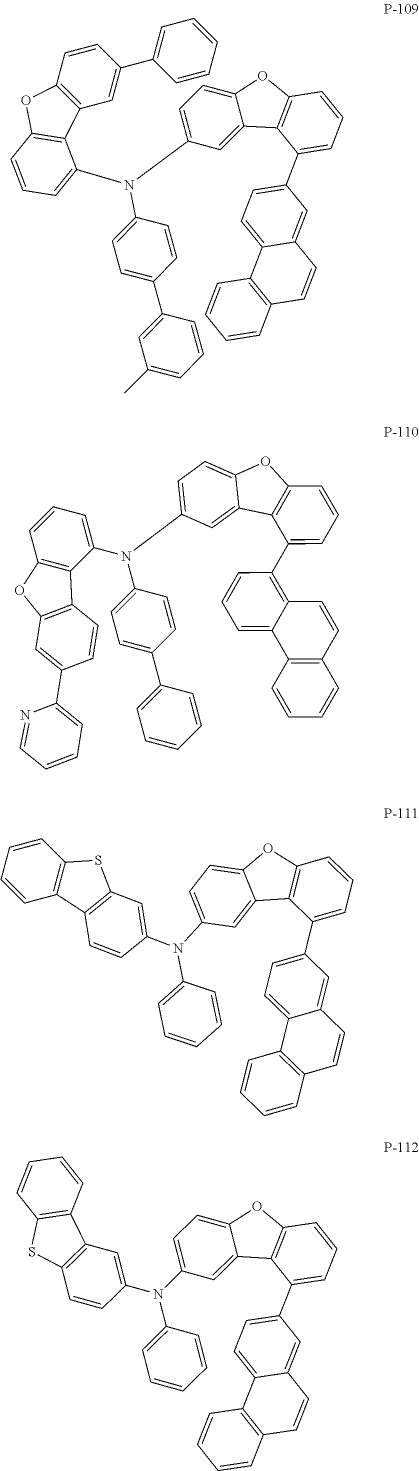 [SMILES: Cc1cccc(-c2ccc(N(c3ccc4oc5cccc(-c6ccc7c(ccc8ccccc87)c6)c5c4c3)c3cccc4oc5ccc(-c6ccccc6)cc5c34)cc2)c1 ; c1ccc(-c2ccc(N(c3ccc4oc5cccc(-c6cccc7c6ccc6ccccc67)c5c4c3)c3cccc4oc5cc(-c6ccccn6)ccc5c34)cc2)cc1 ; c1ccc(N(c2ccc3c(c2)sc2ccccc23)c2ccc3oc4cccc(-c5ccc6c(ccc7ccccc76)c5)c4c3c2)cc1 ; c1ccc(N(c2ccc3sc4ccccc4c3c2)c2ccc3oc4cccc(-c5ccc6c(ccc7ccccc76)c5)c4c3c2)cc1]

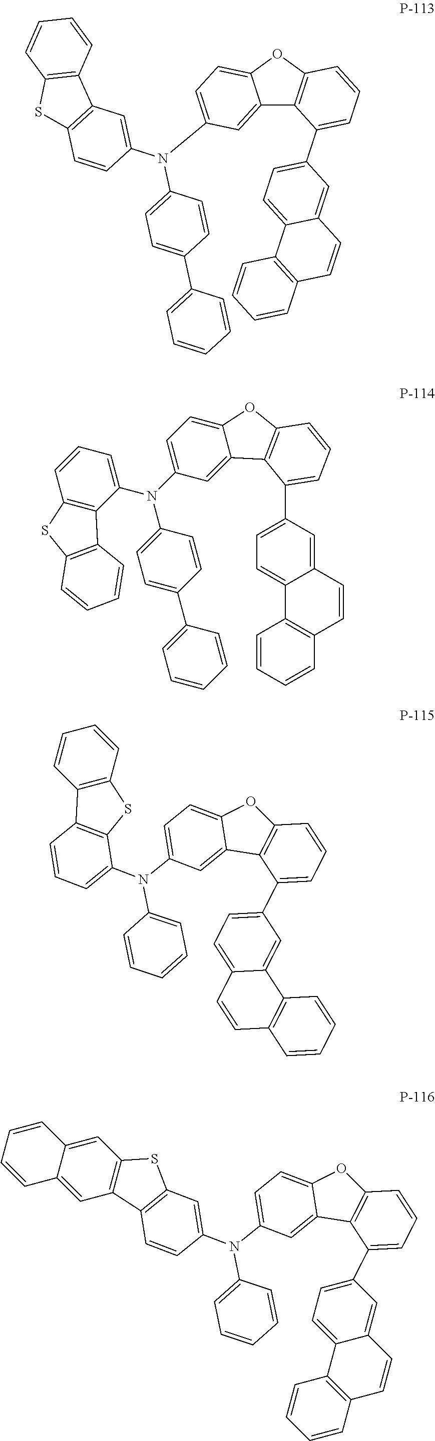 [SMILES: c1ccc(-c2ccc(N(c3ccc4sc5ccccc5c4c3)c3ccc4oc5cccc(-c6ccc7c(ccc8ccccc87)c6)c5c4c3)cc2)cc1 ; c1ccc(-c2ccc(N(c3ccc4oc5cccc(-c6ccc7c(ccc8ccccc87)c6)c5c4c3)c3cccc4sc5ccccc5c34)cc2)cc1 ; c1ccc(N(c2ccc3oc4cccc(-c5ccc6ccc7ccccc7c6c5)c4c3c2)c2cccc3c2sc2ccccc23)cc1 ; c1ccc(N(c2ccc3c(c2)sc2cc4ccccc4cc23)c2ccc3oc4cccc(-c5ccc6c(ccc7ccccc76)c5)c4c3c2)cc1]

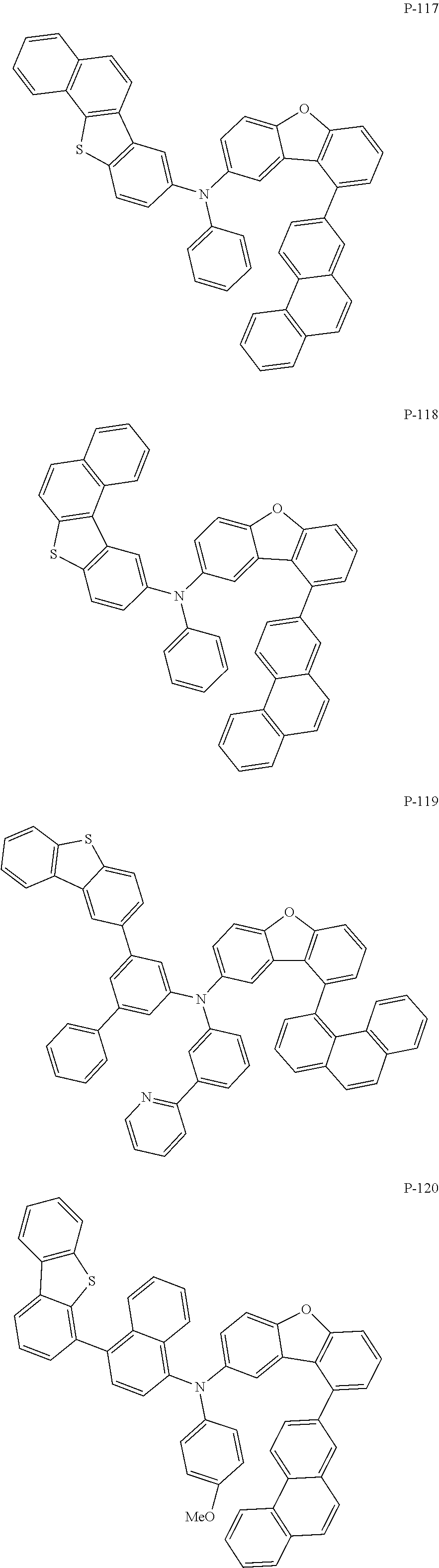 [SMILES: c1ccc(N(c2ccc3sc4c5ccccc5ccc4c3c2)c2ccc3oc4cccc(-c5ccc6c(ccc7ccccc76)c5)c4c3c2)cc1 ; c1ccc(N(c2ccc3oc4cccc(-c5ccc6c(ccc7ccccc76)c5)c4c3c2)c2ccc3sc4ccc5ccccc5c4c3c2)cc1 ; c1ccc(-c2cc(-c3ccc4sc5ccccc5c4c3)cc(N(c3cccc(-c4ccccn4)c3)c3ccc4oc5cccc(-c6cccc7ccc8ccccc8c67)c5c4c3)c2)cc1 ; COc1ccc(N(c2ccc3oc4cccc(-c5ccc6c(ccc7ccccc76)c5)c4c3c2)c2ccc(-c3cccc4c3sc3ccccc34)c3ccccc23)cc1]

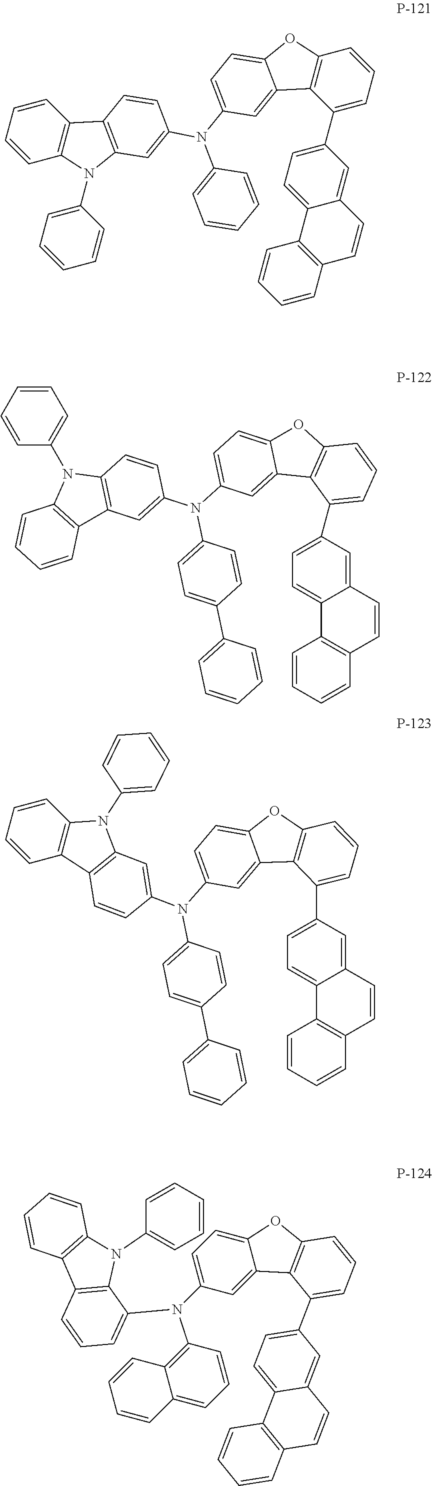 [SMILES: c1ccc(N(c2ccc3oc4cccc(-c5ccc6c(ccc7ccccc76)c5)c4c3c2)c2ccc3c4ccccc4n(-c4ccccc4)c3c2)cc1 ; c1ccc(-c2ccc(N(c3ccc4oc5cccc(-c6ccc7c(ccc8ccccc87)c6)c5c4c3)c3ccc4c(c3)c3ccccc3n4-c3ccccc3)cc2)cc1 ; c1ccc(-c2ccc(N(c3ccc4oc5cccc(-c6ccc7c(ccc8ccccc87)c6)c5c4c3)c3ccc4c5ccccc5n(-c5ccccc5)c4c3)cc2)cc1 ; c1ccc(-n2c3ccccc3c3cccc(N(c4ccc5oc6cccc(-c7ccc8c(ccc9ccccc98)c7)c6c5c4)c4cccc5ccccc45)c32)cc1]

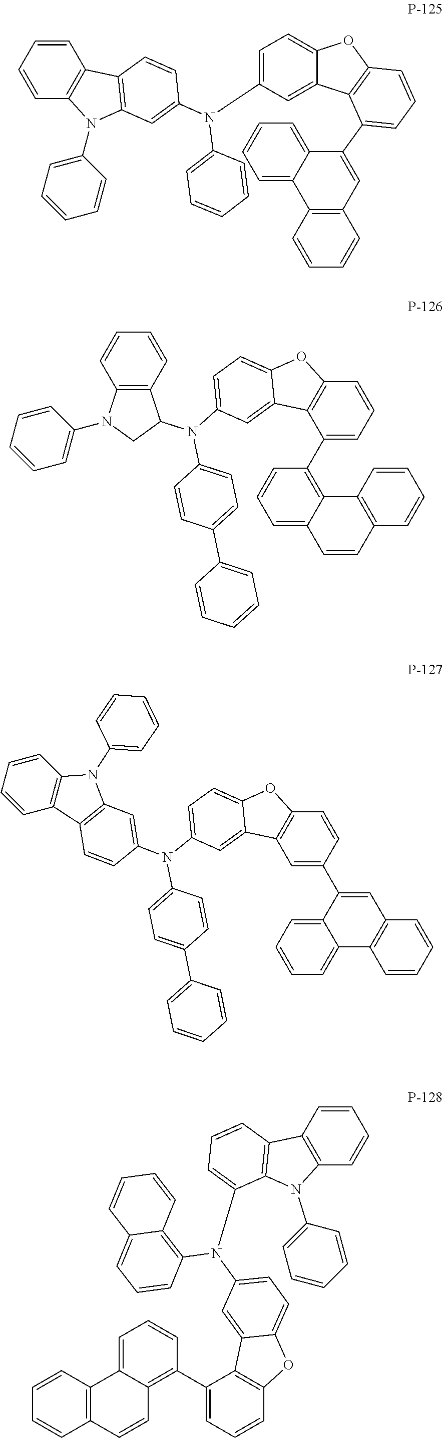 [SMILES: c1ccc(N(c2ccc3oc4cccc(-c5cc6ccccc6c6ccccc56)c4c3c2)c2ccc3c4ccccc4n(-c4ccccc4)c3c2)cc1 ; c1ccc(-c2ccc(N(c3ccc4oc5cccc(-c6cccc7ccc8ccccc8c67)c5c4c3)C3CN(c4ccccc4)c4ccccc43)cc2)cc1 ; c1ccc(-c2ccc(N(c3ccc4oc5ccc(-c6cc7ccccc7c7ccccc67)cc5c4c3)c3ccc4c5ccccc5n(-c5ccccc5)c4c3)cc2)cc1 ; c1ccc(-n2c3ccccc3c3cccc(N(c4ccc5oc6cccc(-c7cccc8c7ccc7ccccc78)c6c5c4)c4cccc5ccccc45)c32)cc1]

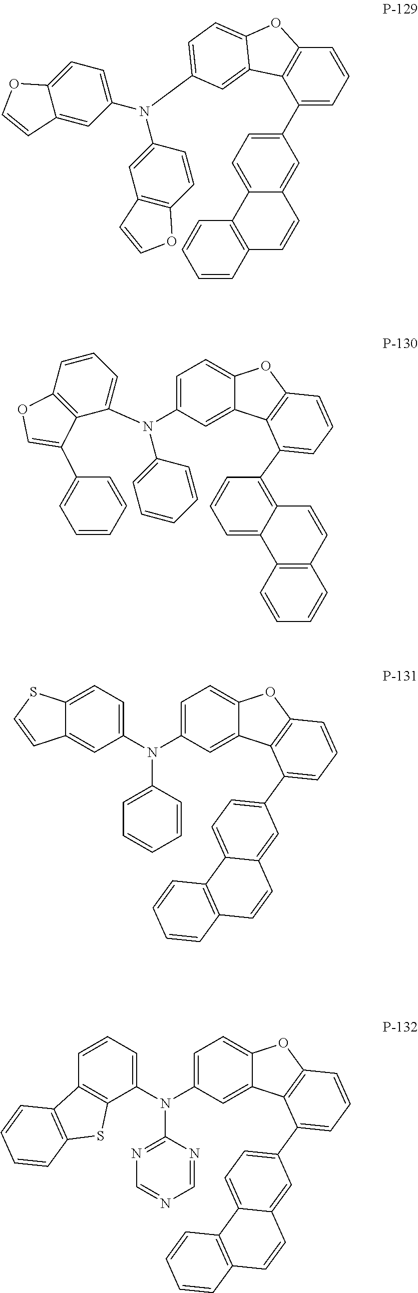 [SMILES: c1ccc2c(c1)ccc1cc(-c3cccc4oc5ccc(N(c6ccc7occc7c6)c6ccc7occc7c6)cc5c34)ccc12 ; c1ccc(-c2coc3cccc(N(c4ccccc4)c4ccc5oc6cccc(-c7cccc8c7ccc7ccccc78)c6c5c4)c23)cc1 ; c1ccc(N(c2ccc3sccc3c2)c2ccc3oc4cccc(-c5ccc6c(ccc7ccccc76)c5)c4c3c2)cc1 ; c1ccc2c(c1)ccc1cc(-c3cccc4oc5ccc(N(c6ncncn6)c6cccc7c6sc6ccccc67)cc5c34)ccc12]

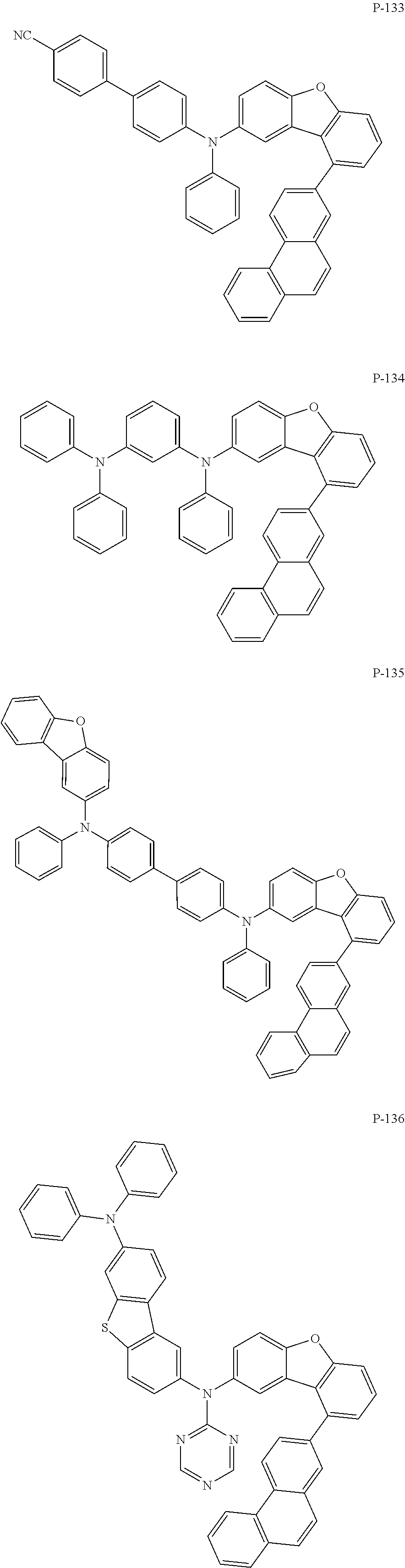 [SMILES: N#Cc1ccc(-c2ccc(N(c3ccccc3)c3ccc4oc5cccc(-c6ccc7c(ccc8ccccc87)c6)c5c4c3)cc2)cc1 ; c1ccc(N(c2ccccc2)c2cccc(N(c3ccccc3)c3ccc4oc5cccc(-c6ccc7c(ccc8ccccc87)c6)c5c4c3)c2)cc1 ; c1ccc(N(c2ccc(-c3ccc(N(c4ccccc4)c4ccc5oc6cccc(-c7ccc8c(ccc9ccccc98)c7)c6c5c4)cc3)cc2)c2ccc3oc4ccccc4c3c2)cc1 ; c1ccc(N(c2ccccc2)c2ccc3c(c2)sc2ccc(N(c4ccc5oc6cccc(-c7ccc8c(ccc9ccccc98)c7)c6c5c4)c4ncncn4)cc23)cc1]

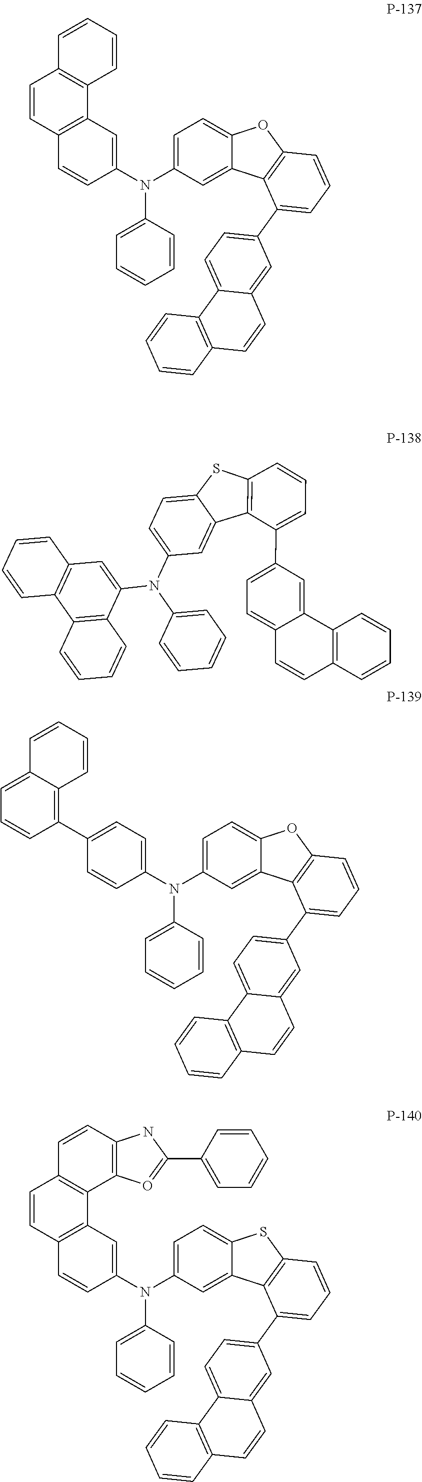

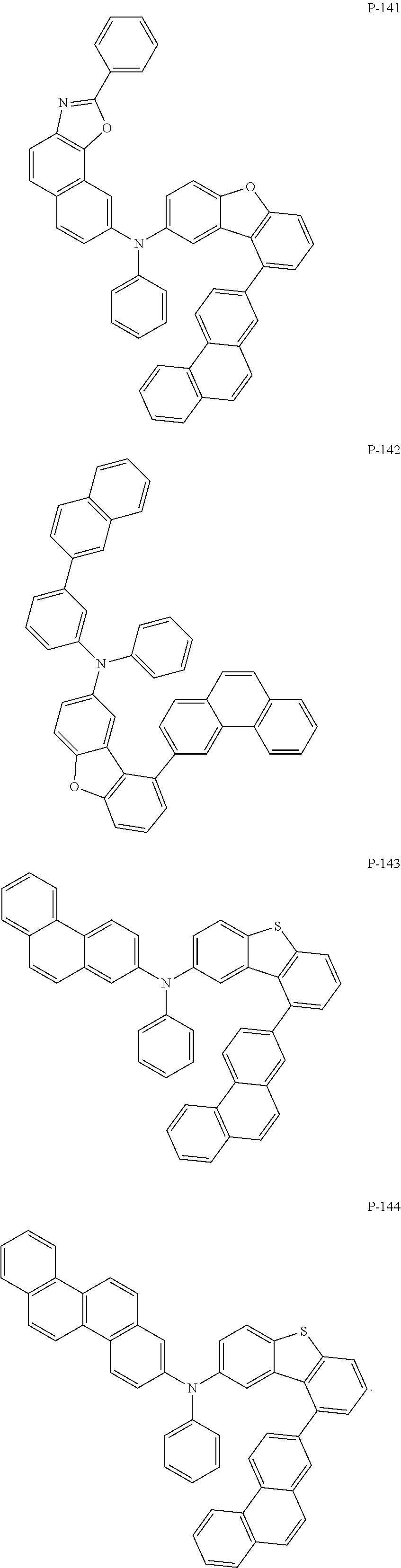 [SMILES: c1ccc(-c2nc3ccc4ccc(N(c5ccccc5)c5ccc6oc7cccc(-c8ccc9c(ccc%10ccccc%109)c8)c7c6c5)cc4c3o2)cc1 ; c1ccc(N(c2cccc(-c3ccc4ccccc4c3)c2)c2ccc3oc4cccc(-c5ccc6ccc7ccccc7c6c5)c4c3c2)cc1 ; c1ccc(N(c2ccc3c(ccc4ccccc43)c2)c2ccc3sc4cccc(-c5ccc6c(ccc7ccccc76)c5)c4c3c2)cc1 ; c1ccc(N(c2ccc3c(ccc4c5ccccc5ccc34)c2)c2ccc3sc4cccc(-c5ccc6c(ccc7ccccc76)c5)c4c3c2)cc1]

7. An organic electric element comprising a first electrode, a second electrode, and an organic material layer between the first electrode and the second electrode, wherein the organic material layer comprises a compound represented by Formula 1 of claim 1.

8. The organic electric element of claim 7, wherein the organic material layer comprises a light emitting layer, and a hole transport zone between the first electrode and the light emitting layer, and the compound represented by Formula 1 is included in at least one of the light emitting layer and the hole transport zone.

9. The organic electric element of claim 8, wherein the hole transport zone comprises an emission-auxiliary layer and the compound represented by Formula 1 is included in the emission-auxiliary layer.

10. The organic electric element of claim 7, wherein the organic electric element further comprises a layer for improving luminous efficiency, wherein the layer for improving luminous efficiency is formed on one side of the first electrode or the second electrode and the one side not facing the organic material layer.

11. The organic electric element of claim 7, wherein the organic material layer comprises two or more stacks each comprising a hole transport layer, a light-emitting layer and an electron transport layer formed sequentially on the first electrode.

12. The organic electric element of claim 11, wherein the stack further comprises an emission-auxiliary layer between the hole transport layer and the light-emitting layer.

13. The organic electric element of claim 11, wherein the organic material layer further comprises a charge generation layer between the two or more stacks.

14. An electronic device comprising a display device and a control unit for driving the display device, wherein the display device comprises the organic electric element of claim 7.

15. The electronic device of claim 14, wherein the organic electric element is selected from the group consisting of an organic electroluminescent element, an organic solar cell, an organic photo conductor, an organic transistor, an element for monochromatic illumination and a quantum dot display.
